(19) United States
(12) Patent Application Publication
(10) Pub. No.: US 2025/0387506 A1
(43) Pub. Date: Dec. 25, 2025

(54) Carborane-Based Boron-Enriched PEG Linkers for mAb Ligation Synthesis and Methods

(71) Applicant: TAE Life Sciences LLC, Foothill Ranch, CA (US)

(72) Inventors: Michael Y. Torgov, Redondo Beach, CA (US); Tioga J. Martin, Los Angeles, CA (US); Jason C. Quintana, Santa Monica, CA (US); Samkeliso Dlamini, Santa Monica, CA (US)

(21) Appl. No.: 19/243,277

(22) Filed: Jun. 19, 2025

Related U.S. Application Data
(60) Provisional application No. 63/662,212, filed on Jun. 20, 2024.

(51) Int. Cl. A61K 47/68; C07D 207/452

(52) U.S. Cl. CPC A61K 47/6889; A61K 47/6849; A61K 47/6851; C07D 207/452

(57) ABSTRACT

Boron Enriched Linker (“BEL”) compounds comprising carborane-based boron enriched PEG linkers and methods of making such BELs are disclosed herein. Consequently, the BELs can be conjugated to antibodies or antibody fragments to create Antibody Boron Conjugates (“ABCs”) to provide a method of treating cancer, immunological disorders, and other disease by utilizing a Neutron Capture Therapy modality.

### CROSS-REFERENCE TO RELATED APPLICATIONS

This application claims priority to U.S. Provisional Patent Application No. 63/662,212 filed 20 Jun. 2024, the contents of which are fully incorporated by reference herein.

### STATEMENT OF RIGHTS TO INVENTIONS MADE UNDER FEDERALLY SPONSORED RESEARCH

Not applicable.

### FIELD OF THE INVENTION

The invention described herein relates to the field of boron neutron capture therapy (BNCT). Specifically, the invention relates to carborane based boron enriched linkers which can be conjugated to a ligand, such as a monoclonal antibody, and used as a vehicle for neutron capture therapy in humans. The invention further relates to the treatment of cancers and other immunological disorders and diseases.

### BACKGROUND OF THE INVENTION

Cancer is the second leading cause of death next to coronary disease worldwide. Millions of people die from cancer every year and in the United States alone cancer kills well over a half-million people annually, with over 1.2 million new cases diagnosed per year (American Cancer Society). While deaths from heart disease have been declining significantly, those resulting from cancer generally are on the rise. In the early part of the next century, cancer is predicted to become the leading cause of death unless medical developments change the current trend.

Several cancers stand out as having high rates of mortality. Carcinomas of the lung, prostate, breast, colon, pancreas, ovary, and bladder represent major causes of cancer death. These and virtually all other carcinomas share a common lethal feature in that they metastasize to sites distant from the primary tumor, and with very few exceptions, metastatic disease is fatal. Moreover, even for those cancer patients who initially survive their primary cancers, common experience has shown that their lives are dramatically altered. Many cancer patients experience strong anxieties driven by the awareness of the potential for recurrence or treatment failure. Many cancer patients also experience physical debilitations following treatment. Furthermore, many cancer patients experience a recurrence of their disease.

Although cancer therapy has improved over the past decades and survival rates have increased, the heterogeneity of cancer still demands new therapeutic strategies utilizing a plurality of treatment modalities. This is especially true in treating solid tumors at anatomical crucial sites (e.g., glioblastoma, squamous carcinoma of the head and neck and lung adenocarcinoma) which are sometimes limited to standard radiotherapy and/or chemotherapy. Nonetheless, detrimental effects of these therapies are chemo- and radio resistance, which promote loco-regional recurrences, distant metastases and second primary tumors, in addition to severe side-effects that reduce the patients' quality of life.

Boron neutron capture therapy (BNCT) is a promising cancer treatment modality that has been underrepresented in both scientific research and clinical application. The primary reasons stem from the disappointing early clinical data in the 1960s and 1970s, which were largely due to technical challenges—such as the lack of effective boron carriers and the limited availability of suitable neutron sources, including nuclear reactors that were not well-equipped for patient treatments. BNCT is a binary treatment modality that relies on boron-10 being present in the tumor at high concentration, and a neutron source that comes in the form of either a research reactor or a particle accelerator. The latter is only now starting to become available. Notably, a borylated amino acid ¹⁰B-L-BPA has been approved for the treatment of head and neck cancers in Japan. As a result of the complexity associated with patient treatment, let alone arranging a proper clinical trial, this treatment modality has been underinvested worldwide. As a result, it is costly because the accelerators that produce neutrons in the energy spectrum suitable for patient treatment are rare, complicated, and are not mass-produced. While there is a resurging interest in BNCT, the deployment of the accelerators is slow and is hampered by the cost to install and operate such a machine. BNCT, being an innovative procedure and associated with real or perceived risks for an early adopter, is likely to contribute further to slow deployment of BNCT in hospitals. Additional challenges include patient selection since there is no established companion diagnostic while the treatment planning is still in its infancy. The latter should be computed based on the unknown parameter—boron concentration in the tumor. Instead, the planning is conducted based on the parameter that can be measured (i.e., boron concentration in the blood and then applying a putative 3:1 tumor-to-blood boron ratio as the foundation for treatment planning). Despite these early setbacks, clinical research from Japan utilizing 18F-FBPA to address both challenges has produced compelling data. Although the number of patients treated was small, the clinical outcomes are highly promising (ZHOU, HIROSE, et. al., Radiotherapy and Oncology 155 (2021) pp. 182-187. In addition to head and neck carcinoma, BNCT has been applied to various cases of melanoma and glioblastoma multiforme (GBM), primarily through investigator-initiated trials that employed nuclear reactors as neutron sources, yielding variable outcomes (See, TAKAI, et. al., Neuro-Oncology, 24(1), pp. 90-98 (2022), and ZHOU, et. al., Am. J. Cancer Res. 2024; 14(2), pp. 429-447.

Stenboronin, 10B-L-BPA, is a non-targeted boron carrier that passively accumulates in the tumor due to an overexpression of LAT-1 (SLC7A5) amino acid transporter. Some research groups have reported promising borylated amino acids with improved tumor retention and capable of achieving higher concentration of boron in the tumor, translating to improved clinical outcomes. See, RAITANO, et. al., J. Med. Chem., https://doi.org/10.1021/acs.jmedchem.3c01265.

Given the current caveats associated with BNCT, it is an object of the present invention to provide new and improved methods of treating cancer(s), immunological disorders, and other diseases utilizing carborane based boron enriched linkers and BNCT.

### SUMMARY OF THE INVENTION

The invention provides for compositions comprising carborane-based Boron Enriched Linkers (BELs) synthesized for use as a delivery modality to treat human diseases such as cancer, immunological disorders, including but not limited to rheumatoid arthritis, ankylosing spondylitis, and other cellular diseases, including but not limited to Alzheimer's disease. In certain embodiments, the BELs comprise one or more Boron clusters operably linked to a ligand, such as an antibody to create an Antibody Boron Conjugate (ABC). In a further embodiment, an ABC of the invention comprises a Boron Antibody Ratio (BAR) from about 12 to several hundred or several thousand.

In a further embodiment, the invention comprises methods of concentrating Boron in a cell comprising (i) synthesizing a BEL; conjugating a BEL of the invention to an antibody, creating an antibody boron conjugate (ABC); (ii) administering the ABC to a patient, and (iii) irradiating the cell with neutrons produced in a neutron source.

In another embodiment, the present disclosure teaches methods of synthesizing BELs.

In another embodiment, the present disclosure teaches methods of synthesizing BELs set forth in FIG. 24.

In another embodiment, the present disclosure teaches methods of synthesizing Compd-7 also referred to as aminooxy-amido-PEG3-Salborin.

In another embodiment, the present disclosure teaches methods of synthesizing Compd-9 also referred to as MC-PEG3-Salborin.

In another embodiment, the present disclosure teaches methods of synthesizing Compd-11 also referred to as Mal-amido-PEG3-Salborin.

In another embodiment, the present disclosure teaches methods of synthesizing Compd-14 also referred to as aminooxy-PEG3-Salborin.

In another embodiment, the present disclosure teaches methods of synthesizing Compd-20 also referred to as Mal-amido-PEG4-EDA-Salborin.

In another embodiment, the present disclosure teaches methods of synthesizing Compd-21 also referred to as Mal-amido-PEG8-EDA-Salborin.

In another embodiment, the present disclosure teaches methods of synthesizing Compd-25 also referred to as Mal-amido-PEG8-aminomethylenecarborane.

In another embodiment, the present disclosure teaches methods of synthesizing Compd-27 also referred to as salborin perfluorophenyl ester.

In another embodiment, the present disclosure teaches methods of synthesizing Compd-31 also referred to as Perfluorophenyl salborin-PEG₃-propanoate.

In another embodiment, the present disclosure teaches methods of synthesizing Compd-7 having a chemical structure set forth in FIG. 25.

In another embodiment, the present disclosure teaches methods of synthesizing Compd-9 having a chemical structure set forth in FIG. 26.

In another embodiment, the present disclosure teaches methods of synthesizing Compd-11 having a chemical structure set forth in FIG. 27.

In another embodiment, the present disclosure teaches methods of synthesizing Compd-14 having a chemical structure set forth in FIG. 28.

In another embodiment, the present disclosure teaches methods of synthesizing Compd-20 having a chemical structure set forth in FIG. 29.

In another embodiment, the present disclosure teaches methods of synthesizing Compd-21 having a chemical structure set forth in FIG. 30.

In another embodiment, the present disclosure teaches methods of synthesizing Compd-25 having a chemical structure set forth in FIG. 31.

In another embodiment, the present disclosure teaches methods of synthesizing Compd-27 having a chemical structure set forth in FIG. 32.

In another embodiment, the present disclosure teaches methods of synthesizing Compd-31 having a chemical structure set forth in FIG. 33.

In another embodiment, the present disclosure teaches a synthetic schema for synthesizing a class of putative Carborane Based Boron Enriched PEG Linkers using the general synthesis set forth in FIGS. 34-52.

In another embodiment, the present disclosure teaches methods of treating cancer(s), immunological disorders, and other diseases in humans.

In another embodiment, the present disclosure teaches methods of treating cancer(s), using boron neutron capture therapy (“BNCT”) in humans.

In another embodiment, the present disclosure teaches methods of treating cancer(s), using proton boron fusion therapy (“PBFT”) in humans.

### BRIEF DESCRIPTION OF THE FIGURES

FIG. 1. Process for Antibody Boron Conjugate (ABC) Preparation.

FIG. 2. Intentionally Omitted.

FIG. 3. Reaction Schema for the Preparation of Compd-A.

FIG. 4. Reaction Schema for the Preparation of Compd-B.

FIG. 5. Reaction Schema for the Preparation of Compd-C.

FIG. 6. Elucidation of the Composition of Compd-B.

FIG. 7. Reverse Phase Analysis of Compd-B.

FIG. 8. Summary and Analytics of Compd-B.

FIG. 9. Characteristics of Compd-B Across Multiple Preparations.

FIG. 10. Purification Analysis of Compd-B-mAbs By Size Exclusion Chromatography (SEC).

FIG. 11. Analytical SEC of Compd-B and mAb Ligation. FIG. 11(A). Shows Compd-B-mAb1.

FIG. 11(B). Shows Compd-B-mAb2.

FIG. 12. Analysis of Polymer-mAb Conjugates via SDS PAGE.

FIG. 13. Method of ABC Preparation.

FIG. 14. Binding Affinity of ABC-4 and mAb-2.

FIG. 15. Characterization of Multiple ABCs via SDS PAGE.

FIG. 16. Boron Uptake and Selectivity of ABC-4 In Vitro.

FIG. 17. Summary of Comp-C-mAb2 Pre-ABC Preparation Characteristics.

FIG. 18. Free Polymer and Aggregation Removal Analysis Using Ceramic Hydroxyapatite Chromatography.

FIG. 19. HMW Removal from Compd.-7-mAb-2.

FIG. 20. Summary of Characteristics for Comp-7-mAb2 After Purification.

FIG. 21. Summary of Characteristics for ABC-4 After Compd.-7 Ligation,

FIG. 22. Affinity Analysis of EGFR on FaDu and MBA-MD-468 Cell Lines. FIG. 22(A). Shows ABC-4 (anti-EGFR) affinity on MDA-MB-468 cell line. FIG. 22(B). Shows ABC-4 (anti-EGFR) affinity on FaDu cell line. FIG. 22(C). Shows boron uptake of ABC-4 across MDA-MB-468 cell line and FaDu cell line.

FIG. 23. Biodistribution Study of ABC-4 at 24 hours. FIG. 23(A). Shows biodistribution at 10 mg/kg in squamous FaDu xenografts. FIG. 23(B). Shows biodistribution at 2, 6 and 24 hrs. following and i.v. injection of ABC-4 (10 mg/kg) in mice bearing squamous AsPC-1 xenografts. FIG. 23(C). Shows EGFR expression by IHC using and-EFGR antibody (top row). Shows bound ABC to AsPC-1 xenografts using anti-human IgG.

FIG. 24. Overview of Compounds of the Disclosure.

FIG. 25. Structure of Compd-7.

FIG. 26. Structure of Compd-9.

FIG. 27. Structure of Compd-11.

FIG. 28. Structure of Compd-14.

FIG. 29. Structure of Compd-20.

FIG. 30. Structure of Compd-21.

FIG. 31. Structure of Compd-25.

FIG. 32. Structure of Compd-27.

FIG. 33. Structure of Compd-31.

FIG. 34. Synthesis Overview of Compd-7,-9,-11.

FIG. 35. Synthesis of Tert-butyl (1-oxo-1-Salborine-5,8,11-trioxa-2-azatridecan-13-yl)carbamate (3.).

FIG. 36. Synthesis of N-(2-(2-(2-(2-aminoethoxy)ethoxy)ethoxy)ethyl) Salbamide (4.).

FIG. 37. Synthesis of Tert-butyl ((1,15-dioxo-1-Salborine-5,8,11-trioxa-2,14-diazahexadecan-16-yl)oxy)carbamate (6.).

FIG. 38. Synthesis of aminooxy-amido-PEG₃-Salborin (Compd-7.).

FIG. 39. Synthesis of MC-PEG₃-Salborin (Compd-9.).

FIG. 40. Synthesis of Mal-amido-PEG₃-Salborin (Compd-11.).

FIG. 41. Synthesis overview of aminooxy-PEG₃-Salborin (Compd-14.).

FIG. 42. Synthesis of aminooxy-carbamate-PEG₃-Salborin (13.).

FIG. 43. Synthesis of aminooxy-PEG₃-Salborin (Compd-14.).

FIG. 44. Synthesis Overview of Compd-20,-21.

FIG. 45. Synthesis of Tert-butyl (2-Salbomidoethyl)carbamate (16.).

FIG. 46. Synthesis of N-(2-aminoethyl) Salbamide (17.).

FIG. 47. Synthesis of Mal-amido-PEG₈-aminomethylenecarborane (Compd-25.).

FIG. 48. Synthesis Overview of Compd-27,-31.

FIG. 49. Synthesis of salborin perfluorophenyl ester (Compd-27).

FIG. 50. Synthesis of Tert-butyl 1-oxo-1-Salborine-5,8,11-trioxa-2-azatetradecan-14-oate (29.).

FIG. 51. Synthesis of 1-oxo-1-Salborine-5,8,11-trioxa-2-azatetradecan-14-oic acid (30.).

FIG. 52. Synthesis of Perfluorophenylsalborin-PEG₃-propanoate (Compd-31.).

FIG. 53. Mass Confirmation of Compd-9 by LCMS.

FIG. 54. Mass Confirmation of Compd-14 by LCMS.

FIG. 55. Mass Confirmation of Compd-20 by LCMS.

FIG. 56. Mass Confirmation of Compd-21 by LCMS.

FIG. 57. Mass Confirmation of Compd-25 by LCMS.

FIG. 58. Mass Confirmation of Compd-27 by LCMS.

FIG. 59. Mass Confirmation of Compd-31 by LCMS.

### DETAILED DESCRIPTION OF THE INVENTION

### Outline of Sections

I.) Definitions
II.) Antibodies
III.) Background of BNCT
IV.) Background of Antibody Boron Conjugates (ABCs)
V.) Boron
   a. Boron Generally
   b. Boron Cluster(s)
VI.) Carborane Based Boron Enriched PEG Linker Compounds
   a. The Stretcher Unit
   b. The Spacer Unit
   c. The Bio-Conjugation Handle
   d. Boron Antibody Ratio (BAR)
      i. Lo-BAR
      ii. Hi-BAR
   e. Markush Formula (I) and Formula (II)
   f. Maleimide PEG ¾ Linker Compounds
      i. Compd-9
      ii. Compd-11
      iii. Compd-20
   g. Oxy-Amine PEG3 Linker Compounds
      i. Compd-7
      ii. Compd-14
   h. Maleimide PEG8 Linker Compounds
      i. Compd-21
      ii. Compd-25
   i. PfP PEG 0/3 Linker Compounds
      i. Compd-27
      ii. Compd-31
VII.) Boron Neutron Capture Therapy Using BELS
VIII.) Methods of Delivering BELs to a Cell
IX.) KITS/Articles of Manufacture

### I.) Definitions

Unless otherwise defined, all terms of art, notations and other scientific terms or terminology used herein are intended to have the meanings commonly understood by those of skill in the art to which this invention pertains unless the context clearly indicates otherwise. In some cases, terms with commonly understood meanings are defined herein for clarity and/or for ready reference, and the inclusion of such definitions herein should not necessarily be construed to represent a substantial difference over what is generally understood in the art.

When a trade name is used herein, reference to the trade name also refers to the product formulation, the generic drug, and the active pharmaceutical ingredient(s) of the trade name product, unless otherwise indicated by context.

The terms “advanced cancer”, “locally advanced cancer”, “advanced disease” and “locally advanced disease” mean cancers that have extended through the relevant tissue capsule, and are meant to include stage C disease under the American Urological Association (AUA) system, stage C1-C2 disease under the Whitmore-Jewett system, and stage T3-T4 and N+ disease under the TNM (tumor, node, metastasis) system. In general, surgery is not recommended for patients with locally advanced diseases and these patients have substantially less favorable outcomes compared to patients having clinically localized (organ-confined) cancer.

The term “antibody” is used in the broadest sense unless indicated otherwise. Therefore, an “antibody” can be naturally occurring or synthetic such as monoclonal antibodies produced by conventional hybridoma technology. Furthermore, antibodies comprise monoclonal and polyclonal antibodies as well as fragments containing the antigen-binding domain and/or one or more complementarity determining regions of these antibodies. As used herein, the term “antibody” refers to any form of antibody or fragment thereof that specifically binds a target antigen and/or exhibits the desired biological activity and specifically covers monoclonal antibodies (Including full length monoclonal antibodies), polyclonal antibodies, multi-specific antibodies (e.g., bispecific antibodies), and antibody fragments so long as they specifically bind a target antigen or fragment thereof and/or exhibit the desired biological activity. Any specific antibody can be used in the methods and compositions provided herein. Thus, in one embodiment the term “antibody” encompasses a molecule comprising at least one variable region from a light chain immunoglobulin molecule and at least one variable region from a heavy chain molecule that in combination form a specific binding site for the target antigen. In one embodiment, the antibody is an IgG antibody. For example, the antibody is an IgG1, IgG2, IgG3, or IgG4 antibody. The antibodies useful in the present methods and compositions can be generated in cell culture, in phage, or in various animals, including but not limited to cows, rabbits, goats, mice, rats, hamsters, guinea pigs, sheep, dogs, cats, monkeys, chimpanzees, and apes, Therefore, in one embodiment, an antibody of the present invention is a mammalian antibody. Phage techniques can also be used to isolate an initial antibody or to generate variants with altered specificity or avidity characteristics. Such techniques are routine and well known in the art. In one embodiment, the antibody is produced by recombinant means known in the art. For example, a recombinant antibody can be produced by transfecting a host cell with a vector comprising a DNA sequence encoding the antibody. One or more vectors can be used to transfect the DNA sequence expressing at least one VL and one VH region in the host cell. Exemplary descriptions of recombinant means of antibody generation and production include Delves, ANTIBODY PRODUCTION: ESSENTIAL TECHNIQUES (Wiley, 1997); Shephard, et al., MONOCLONAL ANTIBODIES (Oxford University Press, 2000); Goding, MONOCLONAL ANTIBODIES: PRINCIPLES AND PRACTICE (Academic Press, 1993); and CURRENT PROTOCOLS IN IMMUNOLOGY (John Wiley & Sons, most recent edition), An antibody of the present invention can be modified by recombinant means to increase the efficacy of the antibody in mediating the desired function. Thus, it is within the scope of the invention that antibodies can be modified by substitutions using recombinant means. Typically, the substitutions will be conservative substitutions. For example, at least one amino acid in the constant region of the antibody can be repla a du e. See, e.g., U.S. Pat. Nos. 5,624,821, 6,194,551, Application No. WO 9958572; and Angal, et al., Mol. Immunol. 30:105-08 (1993). The modification in amino acids incl IS, additions and substitutions of amino acids. In some cases, such changes are made to reduce undesired activities, e.g., complement-dependent cytotoxicity. Frequently, the antibodies are labeled by joining, either covalently or non-covalent substance which provides for a detectable signal. A wide variety of labels and conjugation techniques are known and are reported extensively in both the scientific and patent literature. These antibodies can be screened for binding to normal or defective 168P1D7. See e.g., ANTIBODY ENGINEERING: A PRACTICAL APPROACH (Oxford University Press, 1996). Suitable antibodies with the desired biologic activities can be identified using the following in vitro assays including but not limited to: proliferation, migration, adhesion, soft agar growth, angiogenesis, cell-cell communication, apoptosis, transport, signal transduction, and the following in vivo assays such as the inhibition of tumor growth. The antibodies provided herein can also be useful in diagnostic applications. As capture or non-neutralizing antibodies, they can be screened for the ability to bind to the specific antigen without inhibiting the receptor-binding or biological activity of the antigen. As neutralizing antibodies, the antibodies can be useful in competitive binding assays.

The term “antigen-binding portion” or “antibody fragment” of an antibody (or simply “antibody portion”), as used herein, refers to one or more fragments of an antibody that retain the ability to specifically bind to an antigen. It has been shown that the antigen-binding function of an antibody can be performed by fragments of a full-length antibody. Examples of binding fragments encompassed within the term “antigen-binding portion” of an antibody include (i) a Fab fragment, a monovalent fragment consisting of the V_{L}, V_{H}, C_{L} and C_{H1} domains; (ii) a F(ab′)₂ fragment, a bivalent fragment comprising two Fab fragments linked by a disulfide bridge at the hinge region; (iii) a Fd fragment consisting of the V_{H} and C_{H1} domains; (iv) a Fv fragment consisting of the V_{L} and V_{H}domains of a single arm of an antibody, (v) a dAb fragment (Ward et al., (1989) Nature 341:544-546), which consists of a V_{H} domain; and (vi) an isolated complementarily determining region (CDR). Furthermore, although the two domains of the Fv fragment, V_{L} and V_{H}, are coded for by separate genes, they can be joined, using recombinant methods, by a synthetic linker that enables them to be made as a single protein chain in which the V_{L} and V_{H} regions pair to form monovalent molecules (known as single chain Fv (scFv); see e.g., Bird et al. (1988) Science 242:423-426; and Huston et al. (1988) Proc. Natl. Acad. Sci. USA 85:5879-5883). Such single chain antibodies are also intended to be encompassed within the term “antigen-binding portion” of an antibody. These antibody fragments are obtained using conventional techniques known to those with skill in the art, and the fragments are screened for utility in the same manner as are intact antibodies.

As used herein, any form of the “antigen” can be used to generate an antibody that is specific for the target. Thus, the eliciting of antigen may be a single epitope, multiple epitopes, or the entire protein alone or in combination with one or more immunogenicity enhancing agents known in the art. The eliciting antigen may be an isolated full-length protein, a cell surface protein (e.g., immunizing with cells transfected with at least a portion of the antigen), or a soluble protein (e.g., immunizing with only the extracellular domain portion of the protein). The antigen may be produced in a genetically modified cell. The DNA encoding the antigen may be genomic or non-genomio (e.g., cDNA) and encodes at least a portion of the extracellular domain. As used herein, the term “portion” refers to the minimal number of amino acids or nucleic acids, as appropriate, to constitute an immunogenic epitope of the antigen of interest. Any genetic vectors suitable for transformation of the cells of interest may be employed, including but not limited to adenoviral vectors, plasmids, and non-viral vectors, such as cationic lipids. In one embodiment, the antibody of the methods and compositions herein specifically bind at least a portion of the extracellular domain of the target of interest.

“Antibody Boron Conjugate (“ABC”) is an important class of biopharmaceutical drugs designed as a targeted therapy to enhance Boron Neutron Capture Therapy (BNCT). Unlike ADCs, which consist of antibodies combined with a toxic payload, ABCs are made up of an antibody, or antibody fragment, conjugated with a non-cytotoxic, boron containing molecule such as a Boron Enriched Linker (BEL). It is not until the boron in the ABC is irradiated with epithermal neutrons in a BNCT treatment that it releases a cell killing alpha particle. This type of treatment is currently used in cancer treatment and may also be a suitable for other disease indications. In contrast to chemotherapy and ADC treatment, targeted BNCT using ABCs has the potential to kill only the cancer cells and spare healthy cells. Antibody Boron Conjugates are examples of bioconjugates and immunoconjugates.

“Bispecific” antibodies are also useful in the present methods and compositions. As used herein, the term “bispecific antibody” refers to an antibody, typically a monoclonal antibody, having binding specificities for at least two different antigenic epitopes. In one embodiment, the epitopes are from the same antigen. In another embodiment, the epitopes are from two different antigens. Methods for making bispecific antibodies are known in the art. For example, bispecific antibodies can be produced recombinantly using the co-expression of two immunoglobulin heavy chain/light chain pairs. See, e.g., Milstein et al., Nature 305:537-39 (1983). Alternatively, bispecific antibodies can be prepared using chemical linkage. See, e.g., Brennan, et al., Science 229:81 (1985). Bispecific antibodies include bispecific antibody fragments. See, e.g., Hollinger, et al., Proc. Natl. Acad. Sci. U.S.A. 90:6444-48 (1993), Gruber, et al., J. Immunol, 152:5368 (1994),

“Boronic Acid” means an organic compound related to boric acid (B(OH)₃) in which one of the three hydroxyl groups (—OH) is replaced by an alkyl or aryl group (represented by R in the general formula R—B(OH)₂). As a compound containing a carbon-boron bond, members of this class thus belong to the larger class of organoboranes.

The term “borine” means a compound of one atom of boron and three atoms or molecules of a univalent radical.

The term “borane” also known as borine, is an unstable and highly reactive molecule with the chemical formula BH₃.

“Borylation” means reactions that produce an organoboron compound through functionalization of aliphatic and aromatic C—H bonds.

“Boron Antibody Ratio” (BAR) means the average number of boron atoms conjugated to the antibodies on the creation of antibody boron conjugates (ABCs) using boron enriched linkers (BELs). This is an important attribute of ABCs as the number of borons carried by an antibody, or antibody fragment, and delivered to the tumor cell will directly influence its effectiveness as a treatment. This is because the BAR value affects the efficacy of the drug, as low drug loading reduces the potency, while high drug loading may negatively affect manufacturing properties and pharmacokinetics. For the purposes of this disclosure, the conjugation chemistry taught herein includes, but is not limited to, lysine side chain amidation or cysteine interchain disulfide bond reduction based, resulting in a low BAR (12-60 boron atoms) (Lo-BAR) or a high BAR (>100 boron atoms) (Hi-BAR) can be achieved depending on the design of the BEL being utilized in the conjugation.

“Boron Enriched Linker (BEL) means a component of an Antibody Boron Conjugate (ABC). These are linkers designed to contain a pre-defined number of boron molecules to be used to generate ABCs with a pre-determined number of boron molecules to give a low boron to antibody ratio (lo-BAR) or a high boron to antibody ration (hi-BAR). BELs can be synthesized in multiple formats depending on the number of boron molecules required for attachment to the final ABC. They can be made with either cleavable or non-cleavable linkers and the linkers can be of multiple lengths depending on the ABC requirements and treatment target.

The term “compound” refers to and encompasses the chemical compound (e.g. a BEL) itself as well as, whether explicitly stated or not, and unless the context makes clear that the following are to be excluded: amorphous and crystalline forms of the compound, including polymorphic forms, where these forms may be part of a mixture or in isolation; free acid and free base forms of the compound, which are typically the forms shown in the structures provided herein; isomers of the compound, which refers to optical isomers, and tautomerio isomers, where optical isomers include enantiomers and diastereomers, chiral isomers and non-chiral isomers, and the optical isomers include isolated optical isomers as well as mixtures of optical isomers including racemic and non-racemic mixtures; where an isomer may be in isolated form or in a mixture with one or more other isomers; isotopes of the compound, including deuterium-and tritium-containing compounds, and including compounds containing radioisotopes, including therapeutically-and diagnostically-effective radioisotopes; multimeric forms of the compound, including dimeric, trimerio, etc. forms; salts of the compound, preferably pharmaceutically acceptable salts, including acid addition salts and base addition salts, including salts having organic counterions and inorganic counterions, and including zwitterionic forms, where if a compound is associated with two or more counterions, the two or more counterions may be the same or different; and solvates of the compound, including hemisolvates, monosolvates, disolvates, etc., including organic solvates and inorganic solvates, said inorganic solvates including hydrates; where if a compound is associated with two or more solvent molecules, the two or more solvent molecules may be the same or different. In some instances, reference made herein to a compound of the invention will include an explicit reference to one or of the above forms, e.g., salts and/or solvates; however, this reference is for emphasis only, and is not to be construed as excluding other of the above forms as identified above

The terms “inhibit” or “inhibition of” as used herein means to reduce by a measurable amount, or to prevent entirely.

The term “mammal” refers to any organism classified as a mammal, including mice, rats, rabbits, dogs, cats, cows, horses, and humans. In one embodiment of the invention, the mammal is a mouse. In another embodiment of the invention, the mammal is a human.

The terms “metastatic cancer” and “metastatic disease” mean cancers that have spread to regional lymph nodes or to distant sites and are meant to include stage D disease under the AUA system and stage T×N×M+ under the TNM system.

“Molecular recognition” means a chemical event in which a host molecule is able to form a complex with a second molecule (i.e., the guest). This process occurs through non-covalent chemical bonds, including but not limited to hydrogen bonding, hydrophobic interactions, and ionic interaction.

“Pharmaceutically acceptable” refers to a non-toxic, inert, and/or composition that is physiologically compatible with humans or other mammals,

“Polyhedral Skeletal Electron Pair Theory (PSEPT) means a set of widely accepted electron counting rules useful for predicting the structure of clusters such as borane. The electron counting rules were originally formulated by Kenneth Wade and are sometimes referred to as Wade's rules.

The term “neutron capture agent” means a stable non-reactive chemical isotope which, when activated by neutrons produces alpha-rays and gamma-rays.

The term “neutron capture therapy” means a noninvasive therapeutic modality for treating locally invasive malignant tumors such as primary brain tumors and recurrent head and neck cancer and other immunological disorders and disease by irradiating a neutron capture agent with neutrons.

As used herein, the terms “specific,” “specifically binds” and “binds specifically” refer to the selective binding of the antibody to the target antigen epitope. Antibodies can be evaluated for specificity of binding by comparing binding to appropriate antigen to binding to irrelevant antigen or antigen mixture under a given set of conditions. If the antibody binds to the appropriate antigen at least 2, 5, 7, and preferably 10 times more than to an irrelevant antigen or antigen mixture then it is considered to be specific. In one embodiment, a specific antibody is one that only binds the target antigen but does not bind to the irrelevant antigen. In another embodiment, a specific antibody is one that binds a human target antigen but does not bind a non-human target antigen with 70%, 75%, 80%, 85%, 90%, 91%, 92%, 93%, 94%, 95%, 96%, 97%, 98%, 99% or greater amino acid homology with the human target antigen. In another embodiment, a specific antibody is one that binds a human target antigen and binds a murine target antigen, but with a higher degree of binding the human antigen. In another embodiment, a specific antibody is one that binds a human target antigen and binds a primate target antigen, but with a higher degree of binding the human target antigen. In another embodiment, the specific antibody binds to a human target antigen and any non-human target antigen, but with a higher degree of binding the human antigen or any combination thereof.

As used herein “to treat” or “therapeutic” and grammatically related terms, refer to any improvement of any consequence of disease, such as prolonged survival, less morbidity, and/or a lessening of side effects which are the byproducts of an alternative therapeutic modality; as is readily appreciated in the art, full eradication of disease is a preferred but albeit not a requirement for a treatment act.

“Zwitter-ion” means a molecule with two or more functional groups, of which at least one has a positive and one has a negative electric charge whereby the net charge of the entire molecule is zero. A zwitter-ion is formerly known as a dipolar ion.

### II.) Antibodies

Another aspect of the invention provides antibodies conjugated to BELs of the invention. These antibodies of the invention are particularly useful in cancer prognostic assays, imaging, diagnostic, and therapeutic methodologies. Similarly, such antibodies are useful in the treatment, and/or prognosis of bladder, pancreas, ovarian, head and neck and other cancers, to the extent the target antigen is also expressed or overexpressed in these other cancers. Moreover, antibodies of the invention are therapeutically useful in treating cancers in which the expression of the target antigen is involved and when the antibodies are conjugated to a BEL of the invention as described herein.

Various methods for the preparation of antibodies, specifically monoclonal antibodies, are well known in the art. For example, antibodies can be prepared by immunizing a suitable mammalian host using a target related protein, peptide, or fragment, in isolated or immunoconjugate form (Antibodies: A Laboratory Manual, CSH Press, Eds., Harlow, and Lane (1988); Harlow, Antibodies, Cold Spring Harbor Press, NY (1989)). In addition, fusion proteins of the target can also be used, such as a Target GST-fusion protein. In a particular embodiment, a Target GST fusion protein comprising all or most of the amino acid sequence of the Target is produced, and then used as an immunogen to generate appropriate antibodies.

In addition, naked DNA immunization techniques known in the art are used (with or without purified Target-related protein or expressing cells) to generate an immune response to the encoded immunogen (See, DONNELLY, et. al., 1997, Ann. Rev. Immunol. 15:617-648).

The amino acid sequence of a target protein can be analyzed to select specific regions of the target protein for generating antibodies. For example, hydrophobicity and hydrophilicity analysis of a target amino acid sequence is used to identify hydrophilic regions in the target structure. Regions of a target protein that show immunogenic structure, as well as other regions and domains, can readily be identified using various other methods known in the art, such as Chou-Fasman, Garnier-Robson, Kyte-Doolittle, Eisenberg, Karplus-Schultz, or Jameson-Wolf analysis. Hydrophilicity profiles can be generated using the method of Hopp, T. P. and Woods, K. R., 1981, Proc. Natl. Acad. Sci. U.S.A. 78:3824-3828.

Hydropathicity profiles can be generated using the method of Kyte, J. and Doolittle, R. F., 1982, J. Mol. Biol. 157:105-132. Percent (%) Accessible Residues profiles can be generated using the method of Janin J., 1979, Nature 277:491-492. Average Flexibility profiles can be generated using the method of Bhaskaran R., Ponnuswamy P. K., 1988, Int. J. Pept. Protein Res. 32:242-255. Beta-turn profiles can be generated using the method of Deleage, G., Roux B., 1987, Protein Engineering 1:289-294. Thus, each region identified by any of these programs or methods is within the scope of the present invention. Preferred methods for the generation of target antibodies are further illustrated by way of the examples provided herein. Methods for preparing a protein or polypeptide for use as an immunogen are well known in the art. Also well known in the art are methods for preparing immunogenic conjugates of a protein with a carrier, such as BSA, KLH or other carrier protein. In some circumstances, direct conjugation using, for example, carbodiimide reagents are used; in other instances, linking reagents such as those supplied by Pierce Chemical Co., Rockford, III., are effective. Administration of a target immunogen is often conducted by injection over a suitable time period and with use of a suitable adjuvant, as is understood in the art. During the immunization schedule, titers of antibodies can be taken to determine adequacy of antibody formation.

Target specific monoclonal antibodies can be produced by various means well known in the art. For example, immortalized cell lines that secrete a desired monoclonal antibody are prepared using the standard hybridoma technology of Kohler and Milstein or modifications that immortalize antibody-producing B cells, as is generally known. Immortalized cell lines that secrete the desired antibodies are screened by immunoassay in which the antigen is a Target-related protein. When the appropriate immortalized cell culture is identified, the cells can be expanded, and antibodies produced either from in vitro cultures or from ascites fluid.

Antibodies or fragments of the invention can also be produced by recombinant means. Regions that bind specifically to the desired regions of a Target protein can also be produced in the context of chimeric or complementarity-determining region (CDR) grafted antibodies of multiple species origin. Humanized or human antibodies can also be produced and are preferred for use in therapeutic contexts. Methods for humanizing murine and other non-human antibodies, by substituting one or more of the non-human antibody CDRs for corresponding human antibody sequences, are well known (See, for example, Jones, et. al., 1986, Nature 321:522-525; Riechmann, et. al., 1988, Nature 332:323-327; Verhoeyen, et. al., 1988, Science 239:1534-1536). See also, Carter, et. al., 1993, Proc. Natl. Acad. Sci. USA 89:4285 and Sims, et. al., 1993, J. Immunol. 151:2296.

In a preferred embodiment, the antibodies of the present invention comprise fully human antibodies (Target mAbs). Various methods in the art provide means for producing fully human Target mAbs. For example, a preferred embodiment provides for techniques using transgenic mice, inactivated for antibodyproduction, engineered with human heavy and light chains loci referred to as Xenomouse (Amgen Fremont, Inc.). An exemplary description of preparing transgenic mice that produce human antibodies can be found in U.S. Pat. No. 6,657,103. See also, U.S. Pat. Nos. 5,569,825; 5,625,126; 5,633,425; 5,661,016; and 5,545,806; and Mendez, et. al., Nature Genetics, 15:146-156 (1998); Kellerman, S. A. & Green, L. L., Curr. Opin. Biotechnol 13, 593-597 (2002).

In addition, human antibodies of the invention can be generated using the HuMAb mouse (Medarex, Inc.) which contains human immunoglobulin gene miniloci that encode unrearranged human heavy (mu and gamma) and kappa light chain immunoglobulin sequences, together with targeted mutations that inactivate the endogenous mu and kappa chain loci (See, e.g., Lonberg, et. al., (1994) Nature 368(6474): 856-859).

In another embodiment, fully human antibodies of the invention can be raised using a mouse that carries human immunoglobulin sequences on transgenes and transchromosomes, such as a mouse that carries a human heavy chain transgene and a human light chain transchromosome. Such mice, referred to herein as “KM mice,” such mice are described in Tomizuka, et. al., (2000) Proc. Natl. Acad, Sci. USA 97:722-727 and PCT Publication WO 02/43478 to Tomizuka, et. al.

Human monoclonal antibodies of the invention can also be prepared using phage display methods for screening libraries of human immunoglobulin genes. Such phage display methods for isolating human antibodies are established in the art. See for example: U.S. Pat. Nos. 5,223,409; 5,403,484; and 5,571,698 to Ladner, et. al.; U.S. Pat. Nos. 5,427,908 and 5,580,717 to Dower, et. al.; U.S. Pat. Nos. 5,969,108 and 6,172,197 to McCafferty, et. al.; and U.S. Pat. Nos. 5,885,793; 6,521,404; 6,544,731; 6,555,313; 6,582,915 and 6,593,081 to Griffiths, et. al.

Human monoclonal antibodies of the invention can also be prepared using SCID mice into which human immune cells have been reconstituted such that a human antibody response can be generated upon immunization. Such mice are described in, for example, U.S. Pat. Nos. 5,476,996 and 5,698,767 to Wilson, et. al.

Human monoclonal antibodies of the invention can also be prepared using mice into which genomic sequences bearing endogenous mouse variable segments at the immunoglobulin heavy chain (VH, DH, and JH segments) and/or kappa light chain (VK and JK) loci have been replaced, in whole or in part, with human genomic sequences bearing unrearranged germline variable segments of the human immunoglobulin heavy chain (VH, DH, and JH) and/or kappa light chain (VK and JK) loci (Regeneron, Tarrytown, N.Y.). See, for example, U.S. Pat. Nos. 6,586,251, 6,596,541, 7,105,348, 6,528,313, 6,638,768, and 6,528,314.

In another embodiment, the Target mAb or antigen binding portion thereof competes for binding with an antibody having such heavy and/or light chain CDR(s).

Engineered antibodies of the invention include those in which modifications have been made to framework residues within V_{H} and/or V_{L} (e.g., to improve the properties of the antibody). Typically, such framework modifications are made to decrease the immunogenicity of the antibody. For example, one approach is to “backmutate” one or more framework residues to the corresponding germline sequence. More specifically, an antibody that has undergone somatic mutation may contain framework residues that differ from the germline sequence from which the antibody is derived. Such residues can be identified by comparing the antibody framework sequences to the germline sequences from which the antibody is derived. To return the framework region sequences to their germline configuration, the somatic mutations can be “backmutated” to the germline sequence by, for example, site-directed mutagenesis or PCR-mediated mutagenesis (e.g., “backmutated” from leucine to methionine). Such “backmutated” antibodies are also intended to be encompassed by the invention.

Another type of framework modification involves mutating one or more residues within the framework region, or even within one or more CDR regions, to remove T-cell epitopes to thereby reduce the potential immunogenicity of the antibody. This approach is also referred to as “deimmunization” and is described in further detail in U.S. Patent Publication No. 2003/0153043 by Carr, et. al.

In addition, or alternative to modifications made within the framework or CDR regions, antibodies of the invention may be engineered to include modifications within the Fc region, typically to alter one or more functional properties of the antibody, such as serum half-life, complement fixation, Fc receptor binding, and/or antigen-dependent cellular cytotoxicity. Furthermore, a Target mAb of the invention may be chemically modified (e.g., one or more chemical moieties can be attached to the antibody) or be modified to alter its glycosylation, again to alter one or more functional properties of the mAb. Each of these embodiments is described in further detail below.

In one embodiment, the hinge region of CH1 is modified such that the number of cysteine residues in the hinge region is altered, e.g., increased or decreased. This approach is described further in U.S. Pat. No. 5,677,425 by Bodmer, et. al. The number of cysteine residues in the hinge region of CH1 is altered to, for example, facilitate assembly of the light and heavy chains or to increase or decrease the stability of the Target mAb.

In another embodiment, the Fo hinge region of an antibody is mutated to decrease the biological half-life of the Target mAb. More specifically, one or more amino acid mutations are introduced into the CH2-CH3 domain interface region of the Fo-hinge fragment such that the antibody has impaired Staphylococcyl protein A (SpA) binding relative to native Fc-hinge domain SpA binding. This approach is described in further detail in U.S. Pat. No. 6,165,745 by Ward, et. al.

In another embodiment, the Target mAb is modified to increase its biological half-life. Various approaches are possible. For example, mutations can be introduced as described in U.S. Pat. No. 6,277,375 to Ward. Alternatively, to increase the biological half-life, the antibody can be altered within the CH1 or CL region to contain a salvage receptor binding epitope taken from two loops of a CH2 domain of an Fc region of an IgG, as described in U.S. Pat. Nos. 5,869,046 and 6,121,022 by Presta, et. al.

In yet other embodiments, the Fc region is altered by replacing at least one amino acid residue with a different amino acid residue to alter the effector function(s) of the Target mAb. For example, one or more amino acids selected from amino acid specific residues can be replaced with a different amino acid residue such that the antibody has an altered affinity for an effector ligand but retains the antigen-binding ability of the parent antibody. The effector ligand to which affinity is altered can be, for example, an Fo receptor or the C1 component of complement. This approach is described in further detail in U.S. Pat. Nos. 5,624,821 and 5,648,260, both by Winter, et. al.

Reactivity of Target antibodies with a Target-related protein can be established by a number of well-known means, including Western blot, immunoprecipitation, ELISA, and FACS analyses using, as appropriate, Target-related proteins, Target-expressing cells or extracts thereof. A Target mAb or fragment thereof can be labeled with a detectable marker or conjugated to a second molecule. Suitable detectable markers include, but are not limited to, a radioisotope, a fluorescent compound, a bioluminescent compound, chemiluminescent compound, a metal chelator, or an enzyme. Further, bi-specific antibodies specific for two or more target epitopes are generated using methods generally known in the art. Homodimeric antibodies can also be generated by cross-linking techniques known in the art (e.g., Wolff, et. al., Cancer Res. 53:2560-2565).

One of ordinary skill in the art will appreciate and be enabled to make variations and modifications to the disclosed embodiment without altering the function and purpose of the invention disclosed herein. Such variations and modifications are intended within the scope of the present disclosure.

### III.) Background of Boron Neutron Capture Therapy (BNCT)

As previously discussed, BNCT is a promising cancer treatment modality. BNCT is a binary treatment modality that relies on boron-10 being present in the tumor at high concentration, and a neutron source that comes in the form of either a research reactor or a particle accelerator. A borylated amino acid 10B-L-BPA has been approved for the treatment of head and neck cancers in Japan. Notably, as a result of the complexity associated with patient treatment and the administrative burden with conducting a proper clinical trial, this treatment modality has been significantly underinvested worldwide. While there is a resurging interest in BNCT, the deployment of the accelerators is slow and is hampered by the excessive cost to install and operate such a machine. Additional challenges include patient selection, since at this time, there is no established companion diagnostic while the treatment planning is still in its preliminary stages. The latter should be computed based on the unknown parameter—boron concentration in the tumor. Instead, however, the planning is carried out based on a parameter that can be measured (i.e., boron concentration in the blood and then applying a putative 3:1 tumor-to-blood boron ratio as the foundation for treatment planning). Despite this, data from Japanese clinical research that utilized 18F-FBPA to help address both of above factors is compelling while the clinical outcomes albeit with a small number of patients treated are very promising (See, HIROSE, et. al.).

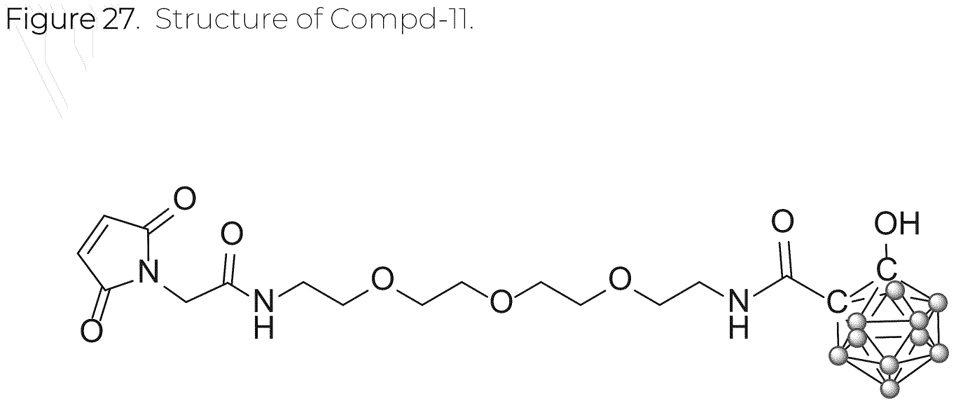

In view of the aforementioned, the following Carborane Based Boron Enriched PEG Linkers are disclosed herein and more fully referenced in FIGS. 27 to 33. Further description and characterization of the compounds is set forth herein.

### IV.) Background of Antibody Boron Conjugates (ABCs)

The idea of using an antibody to deliver boron to the tumor in a targeted fashion has been tested by a small number of research groups. The late 1980's is considered the dawn of monoclonal antibodies when hybridoma techniques were invented. Along with that came the idea of a “silver bullet” for cancer treatment in the form of an Antibody drug conjugate (ADC). Through the lens of ADC's, an idea of targeted boron delivery with a BNCT potential was crystallized. Early reports describe the preparation of conjugates comprised of a poly-L-ornithine or poly-L-lysine scaffold for attaching boron cages. The polymer sidechains were first alkylated with a succinimidyl 3-(2-pyridyldithio) propionate i.e., SPDP bifunctional cross-linker followed by a thiol-disulfide exchange to attach [B₁₂H₁₁SH]₂-inorganic boron cage (i.e., BSH). The polymer was then ligated to a 225.28S antibody directed against a melanoma-associated antigen using a photo cross-linking technique. Up to 1700 boron per mAb was estimated to be achievable before the loss of immunoreactivity although there are significant gaps in the characterization of resulting borylated polyaminoacid-antibody conjugate. See, TAMAT, et. al., Pigment Cell Res. 2: pp. 278-280 (1989). Another group used synthetic isocyanate-derivatized boron clusters comprised of either [B₁₂H₁₁NCO]₂— or [N(CH₃)₃B₁₀C₈NCO]₁— cages to alkylate poly-LD-lysine resulting in a borono-poly-LD-lysine (i.e., BPL). The latter had a trimethylammonia group that offsets the net negative charge of the cage. See, ALAM, et. al., J. Med. Chem., 1989, 32, pp. 2326-2330. The dianion-containing BPL was incompatible with antibody conjugation and caused mAb precipitation following simple admixture. The mono-anion cage was more suitable for subsequent ligation to antibody. Thus, borylated macromolecule was conjugated to an IB16.6 monoclonal antibody targeted against B16 melanoma by first modifying a small number of lysine residues with SPDP. After reducing the disulfide, BPL-SPDP was added to maleimide-modified.antibody. Upon combining the above macromolecules and removing the unconjugated BPL by gel-filtration, a borylated antibody-polylysine conjugate was produced that contained 2700 boron atoms and retained immunoreactivity that was estimated by ELISA. Notably, other borylated antibodies were attempted as well using the above method. However, immunoreactivity was inferior in most preparations.

In another report from the same group, a trimethylammonia polyhedral cluster noted above, [N(CH₃)3B₁₀C₈NCO]₁— was applied to acylate a “startburst” PAMAM dendrimer followed by ligation to cetuximab, See, WU, et. al., Bioconjugate Chem. (2004), 15, pp. 185-194. The PAMAM-Cetuxomab complex was isolated by Sephacryl S300 and radio-lodinated in order to estimate immunoreactivity. It showed saturable dose-response when incubated with EGFR-expressing F98 rat glioma cells. A boron localization and biodistribution using brain orthotopic F98 glioma model following intra-brain injection of the conjugate revealed boron accumulation in both wt and EGFRvIII tumors but not when dendrimer alone was injected, suggesting that the tumor uptake is specific. Boron concentration of over 90 μg/g tissue was measured at 24 hr. in EGFRvIII tumor while the surrounding normal right and left brain had 15±5 μg/g tissue. In the control arm the borylated dendrimer alone resulted in the tumor boron content of 6 μg/g at 24 hr, which was 13.8-fold less than the cetuximab targeted borylated dendrimer.

A different approach to deliver boron via antibody was undertaken by the Hawthorne group. Notably, a bispecific antibody raised against CEA and a set of nido-carborane compounds were generated by fusing two hybridomas producing anti-CEA mAbs and anti-nido-carborane mAbs and evaluated both in vitro and in vivo. See, PRIMUS, et. al., Bioconjugate Chem. 1996, 7, pp. 532-535. BsMAB was extensively purified to remove the parent antibody and following radioiodination, its tumor uptake was compared to the parental anti-CEA antibody in athymic nude mice bearing LS-174T colon carcinoma xenograft. The disappearance of 125I from the tumor was faster than for the control parental antibody. Bispecificity was measured by the ability to deliver radio-iodinated nido-carborane molecule using LS-174T cell line. The signal was only detected upon incubation of BsMAB with the cells and not in the absence of BsMAB. Likewise, signal due to radio-iodinated carborane was not detected in the CEA-negative SK-BR-3 cell line. Thus, there is a potential for pre-targeting using these BsMAB followed by the administration of a carborane-polymer.

The above pre-targeting approach is also described in a patent application by Immunomedics (See, U.S. Pat. No. 6,228,362). In brief, this work describes a bispecific antibody raised against both CEA and carborane. The mAb can be administered first and allowed to clear from the blood. A borylated dextran is administered next to be captured by the cancer cell-bound antibody. The above method of pre-targeting is claimed to include the time window of administration of the borylated polymer. Interestingly, the methods of preparation of allyl-modified dextran as well as the methods of producing a 70 kDa dextrane that can be borylated by reacting BSH (sodium borocaptate dianion) with allyl groups on the polymer are not claimed.

A similarly prepared dextran-BSH polymer that can be conjugated to anti-EGFR Fab binding fragment by per-iodide oxidation of random dextran units followed by reductive amination is described in the patent from Tenboron (See, U.S. 2017/0112931).

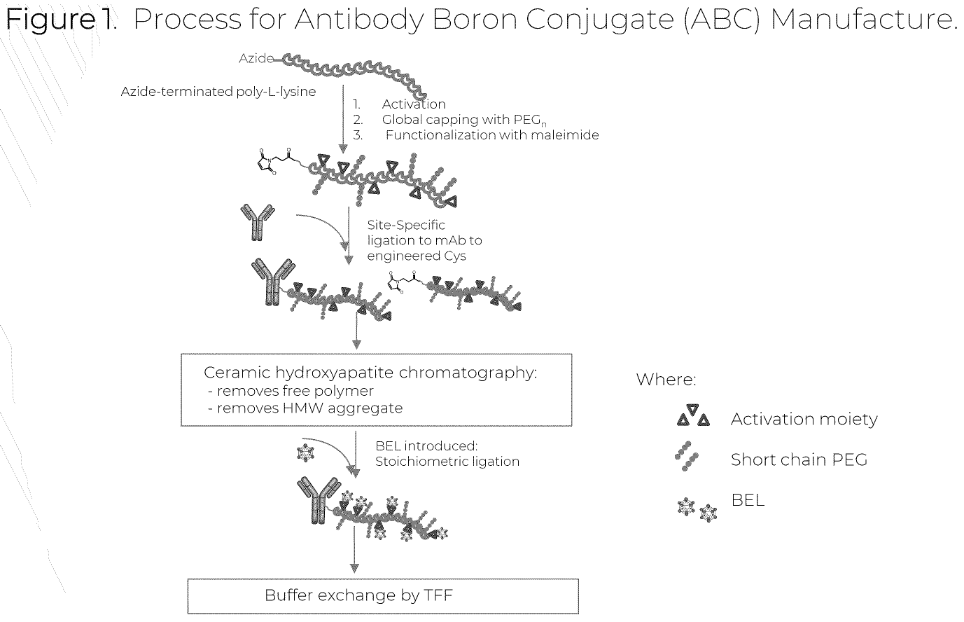

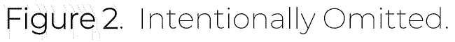

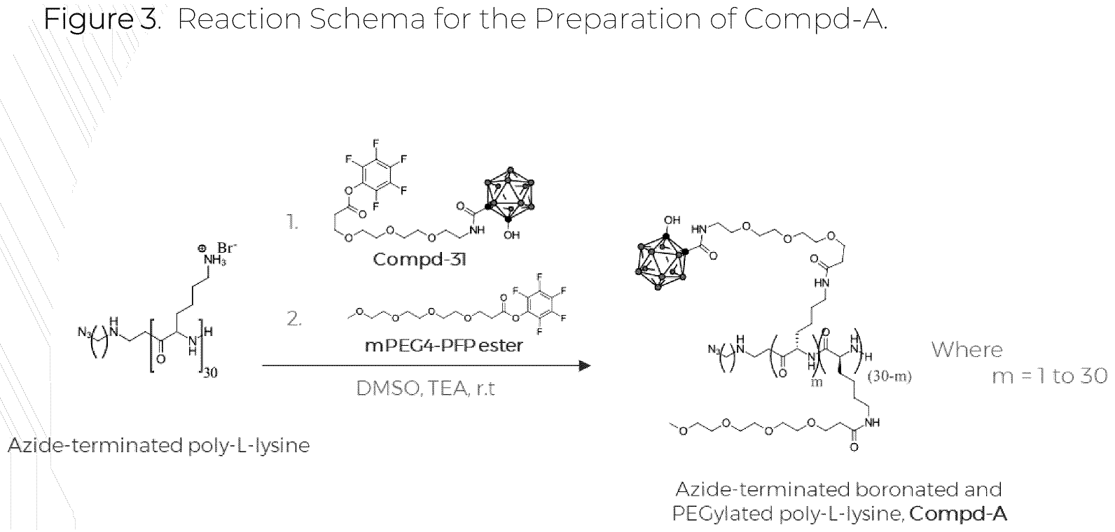

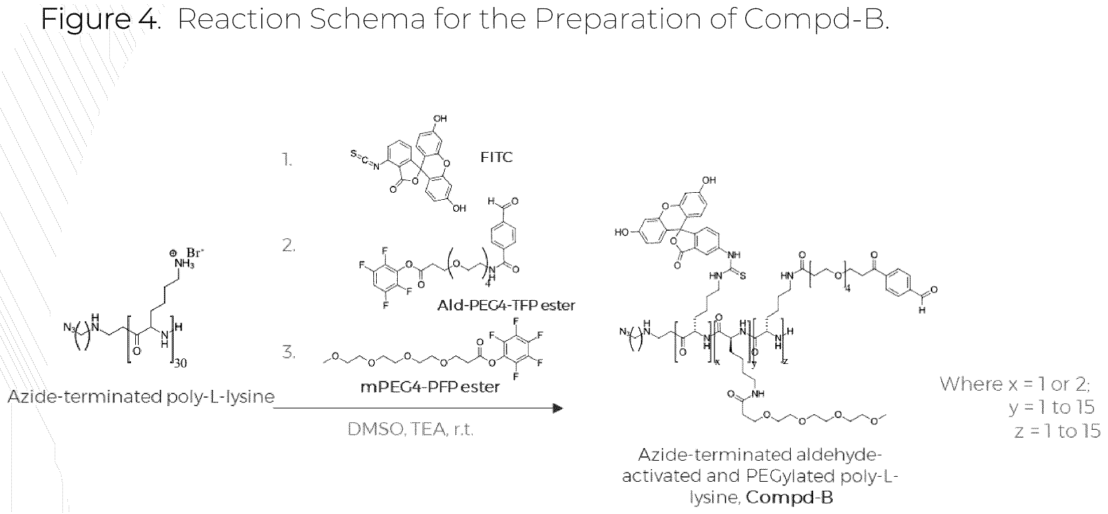

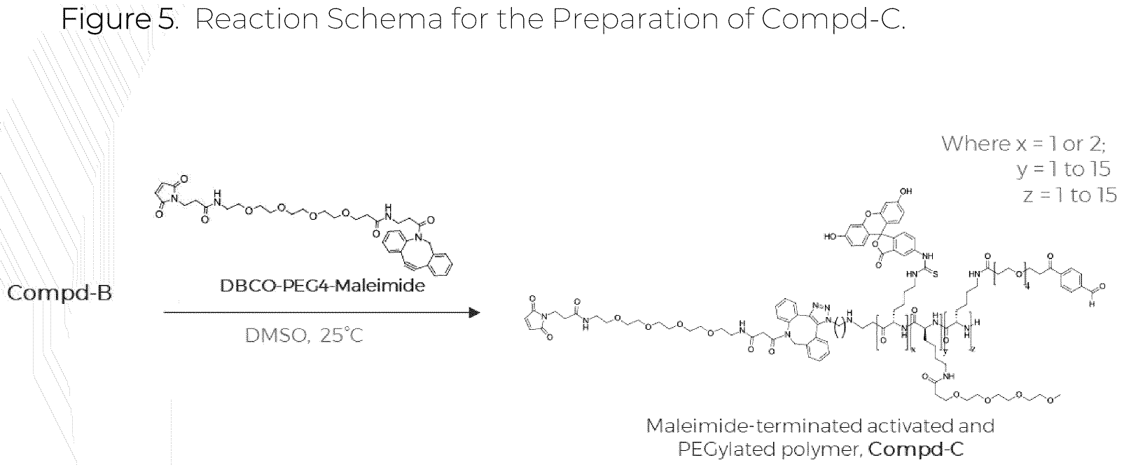

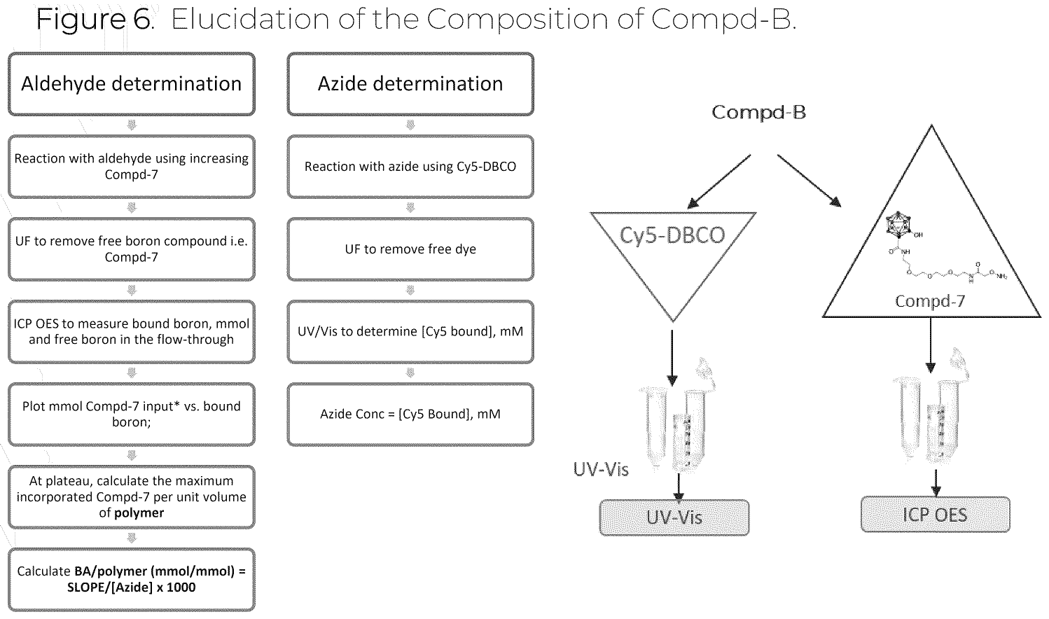

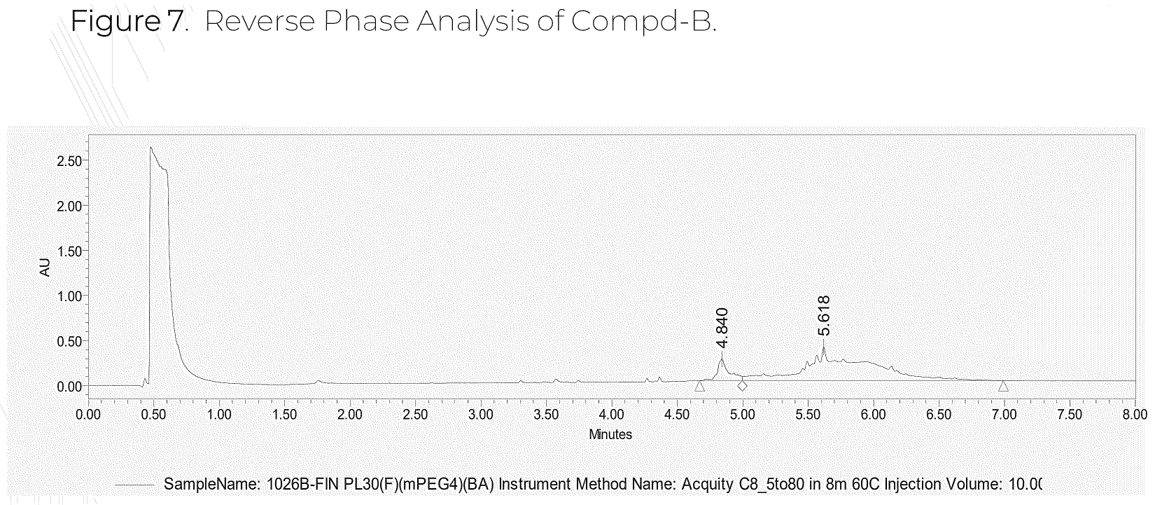

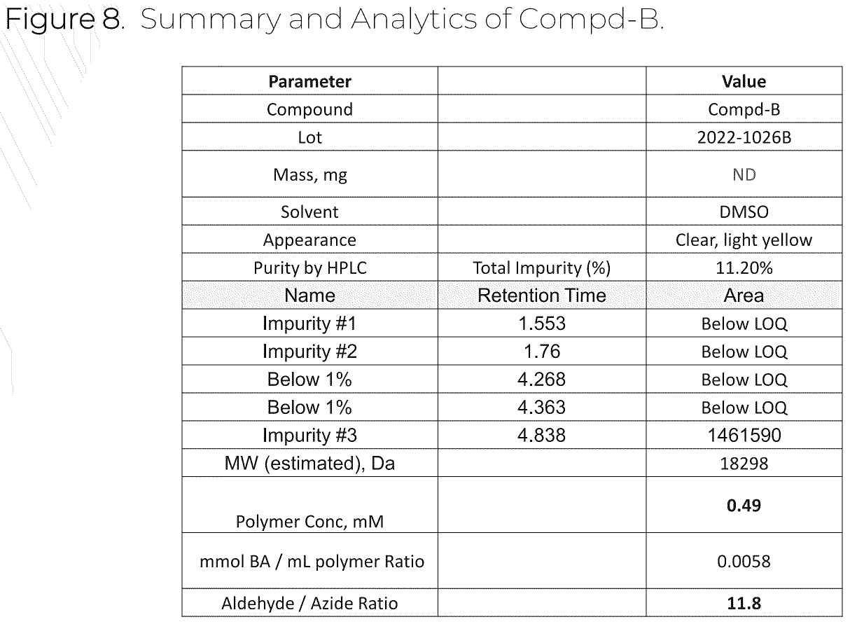

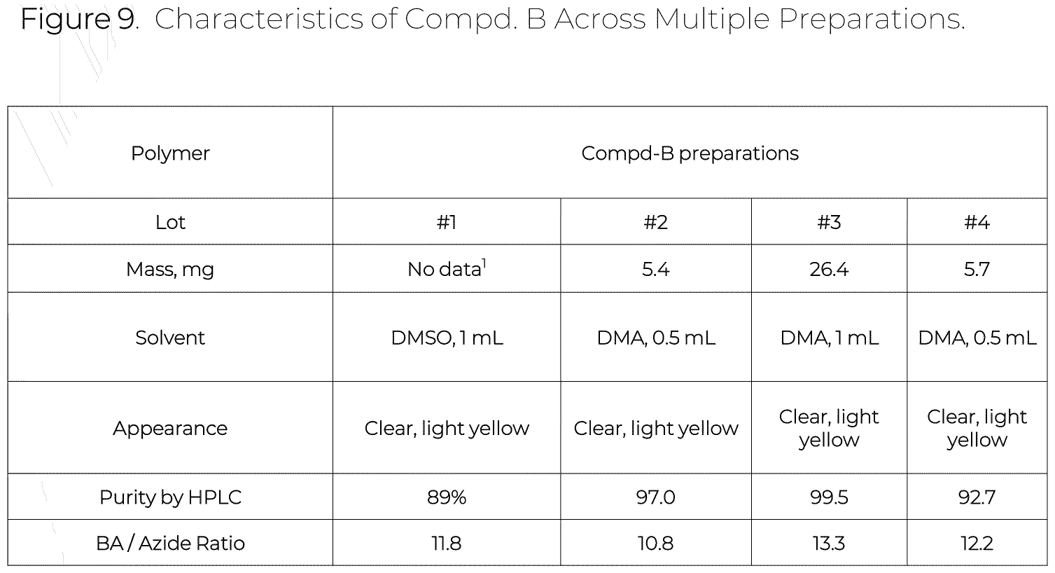

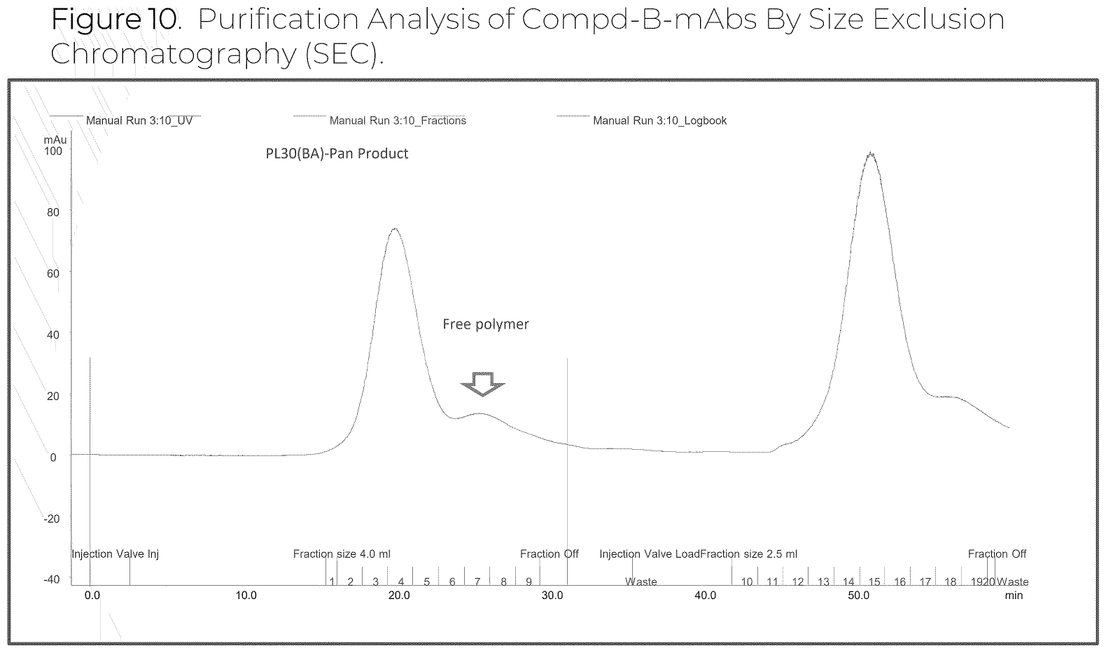

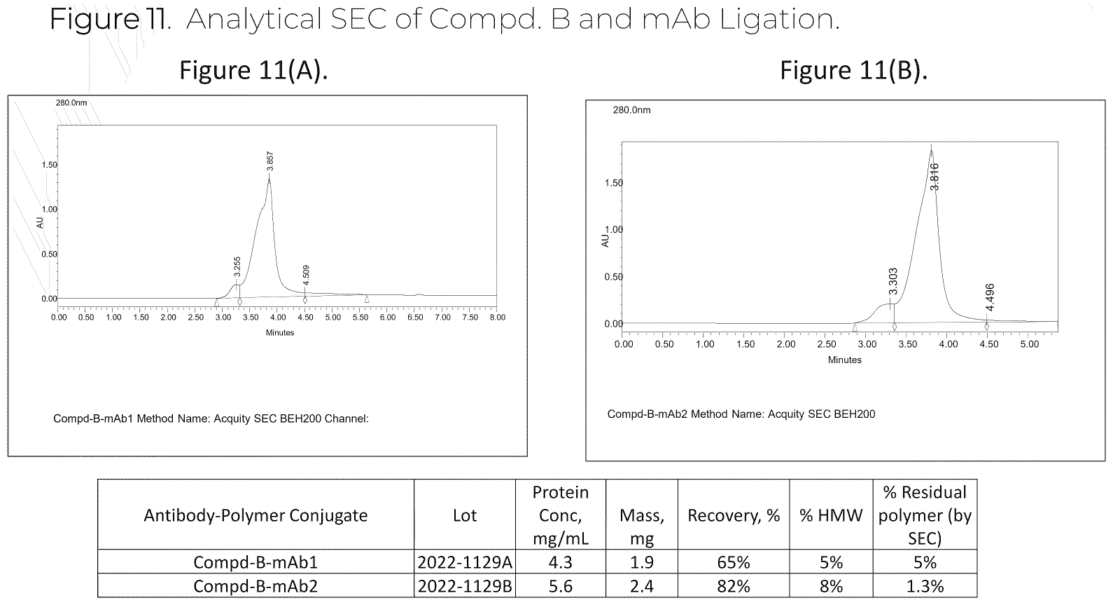

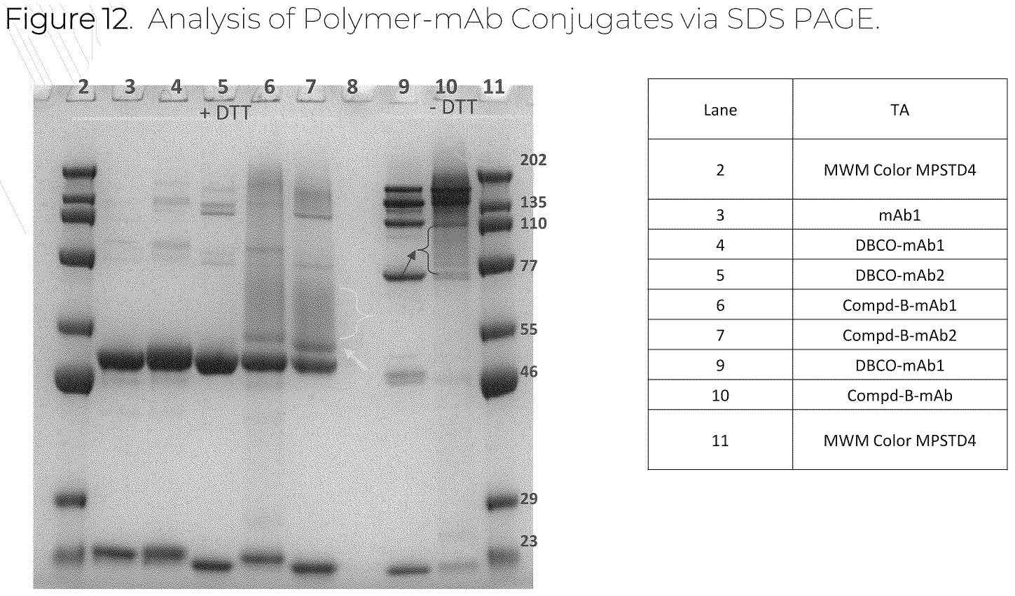

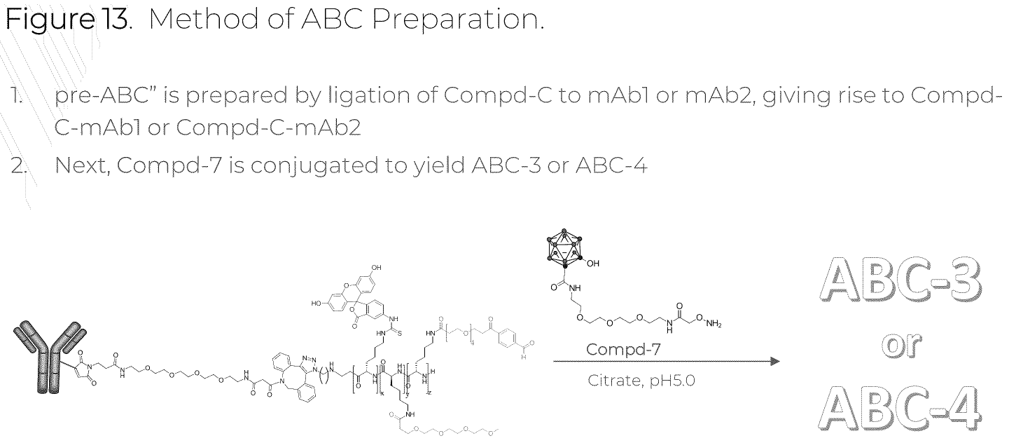

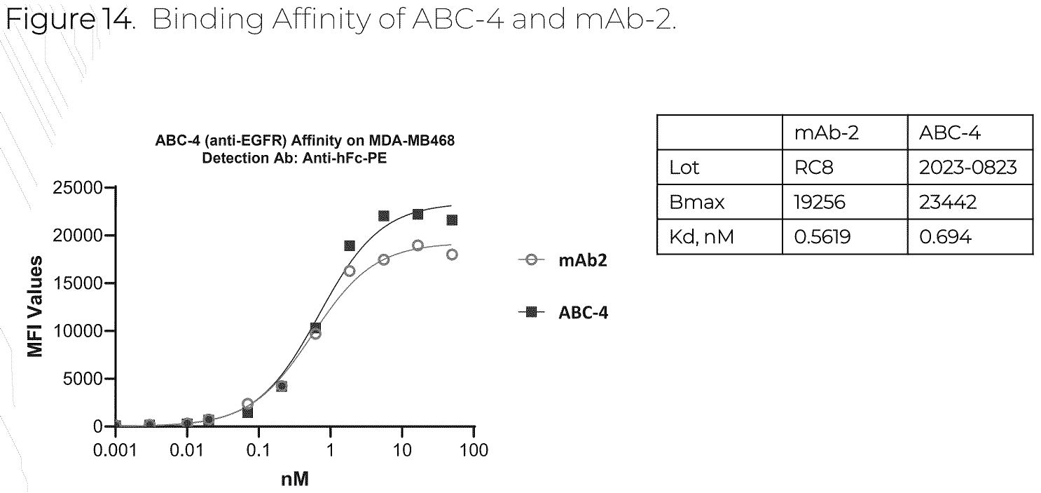

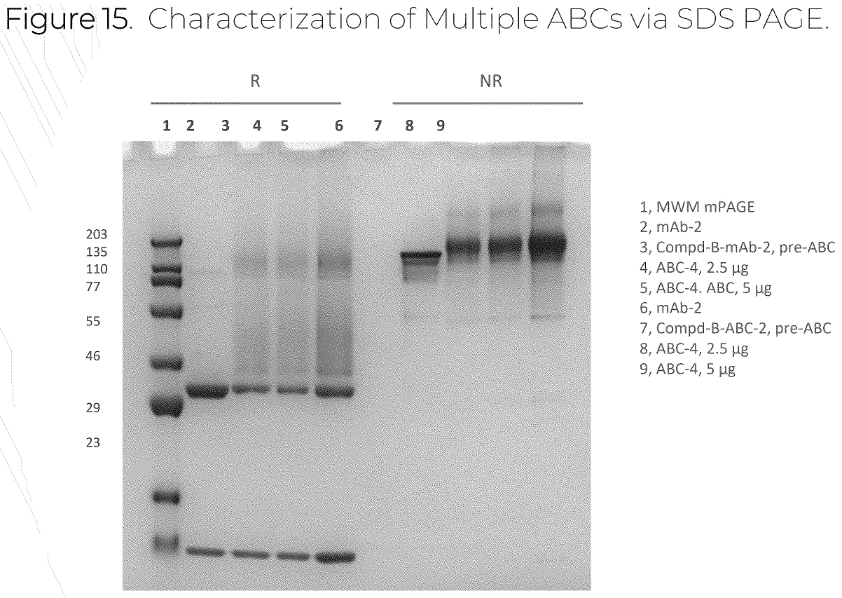

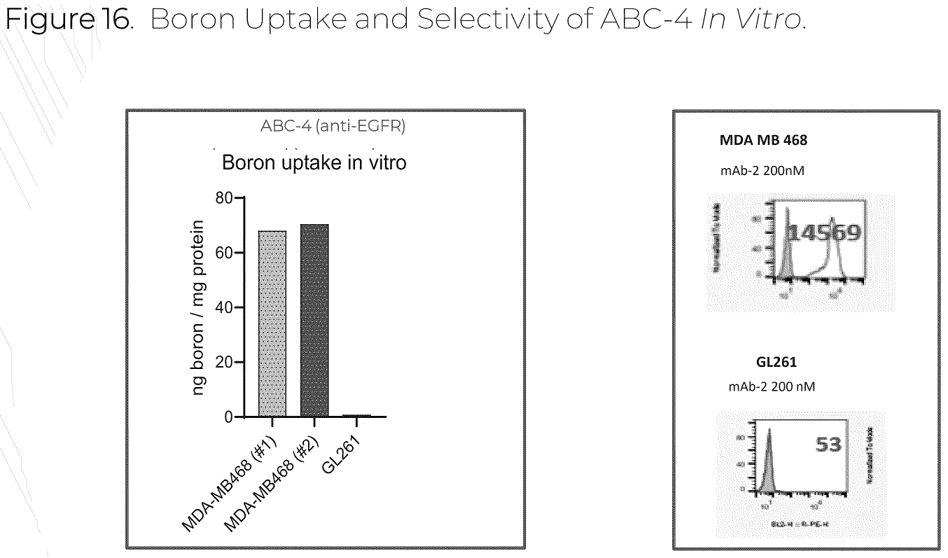

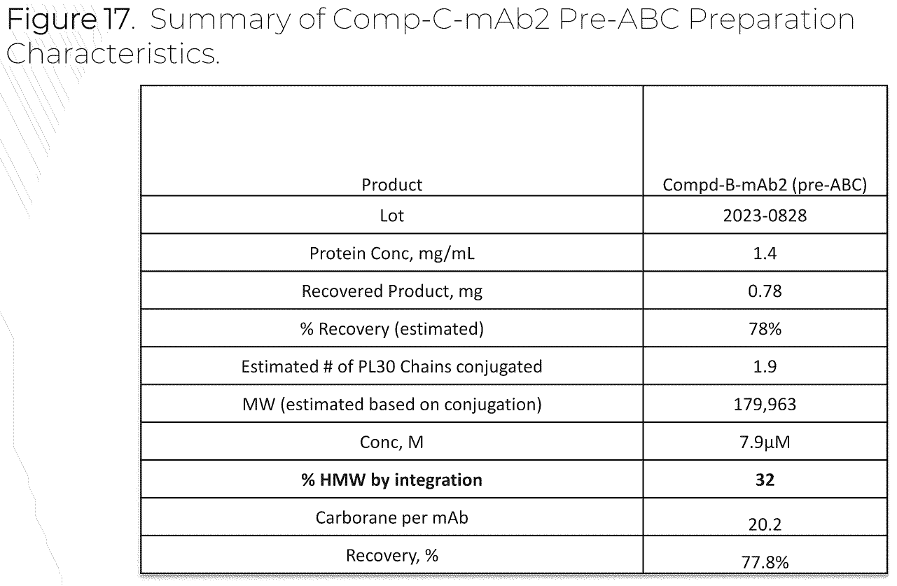

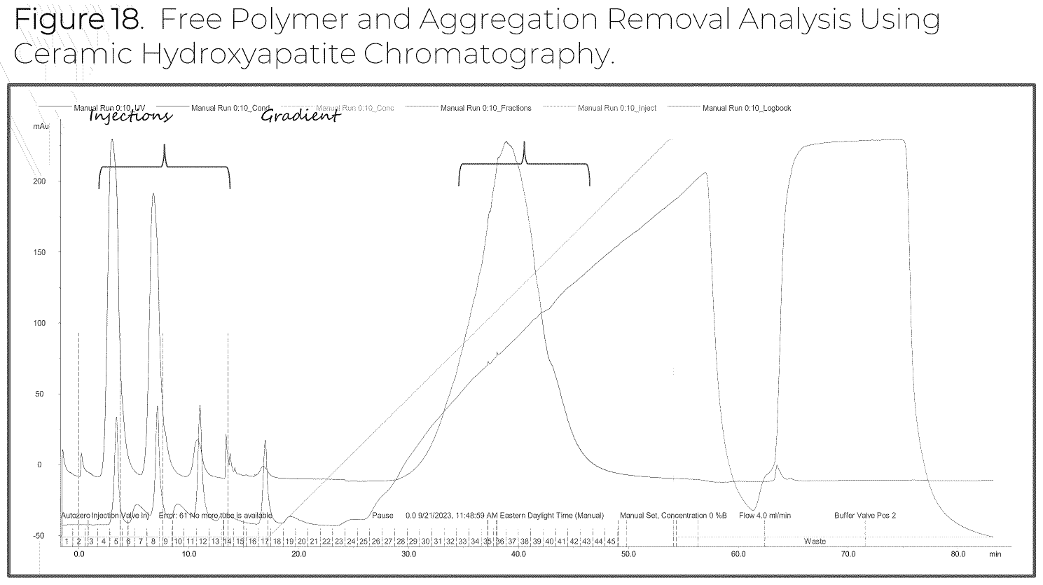

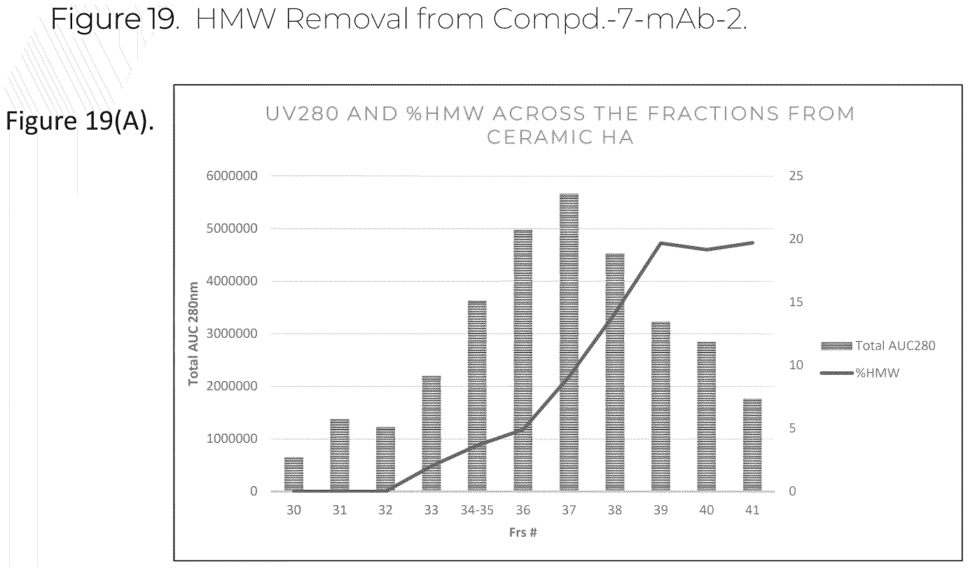

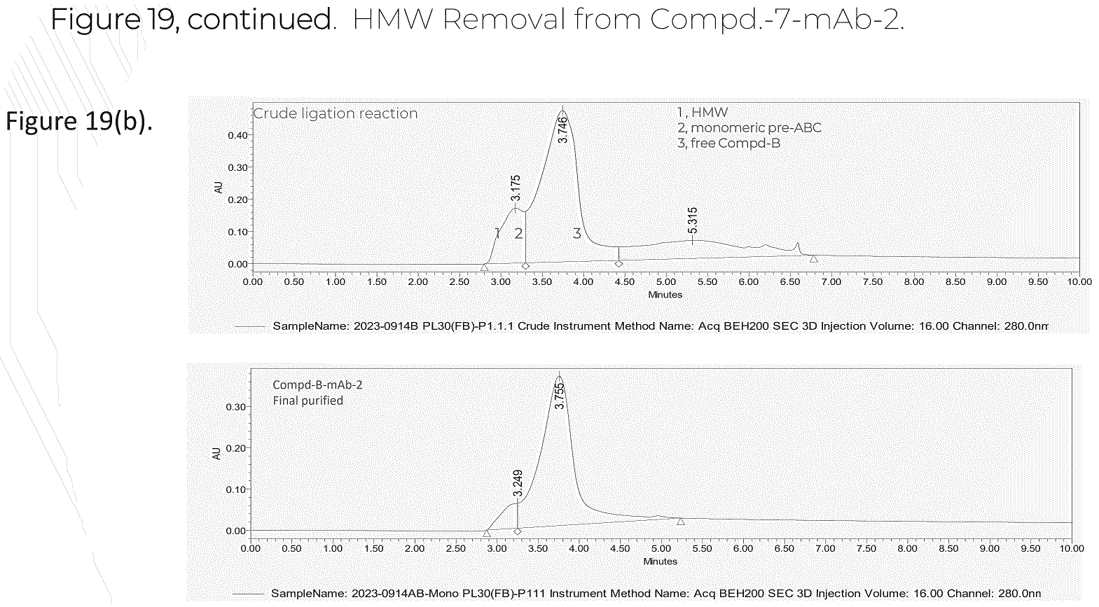

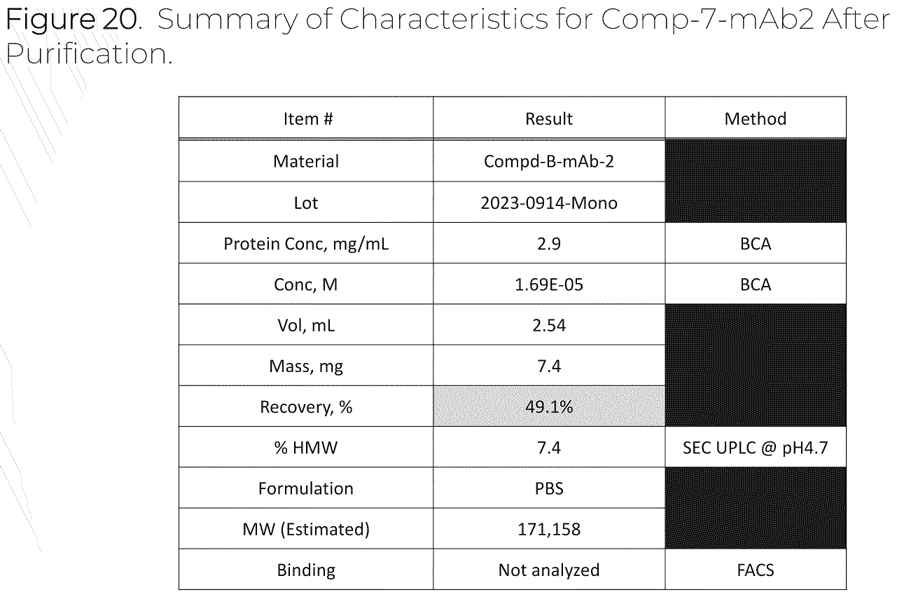

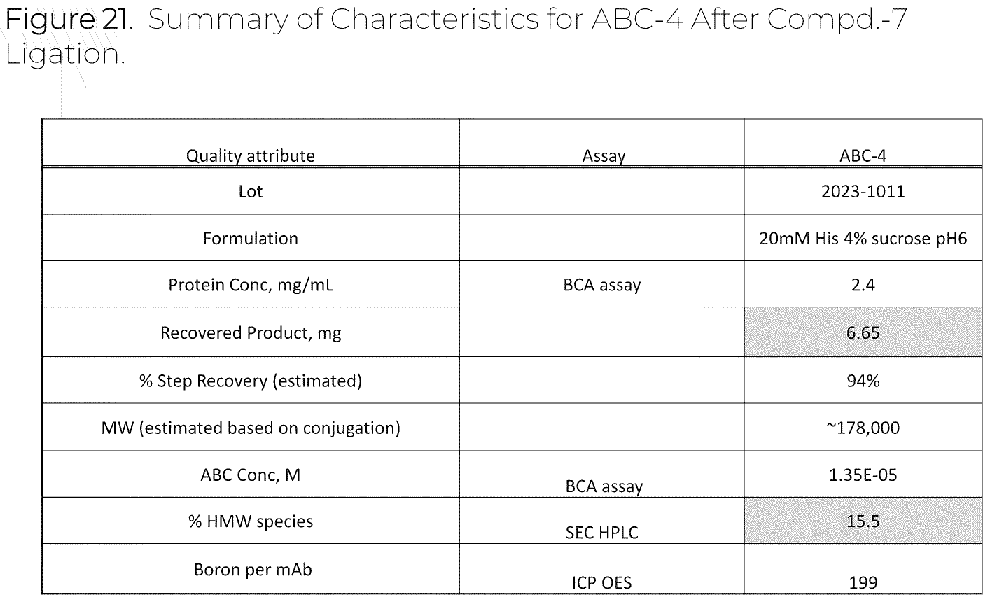

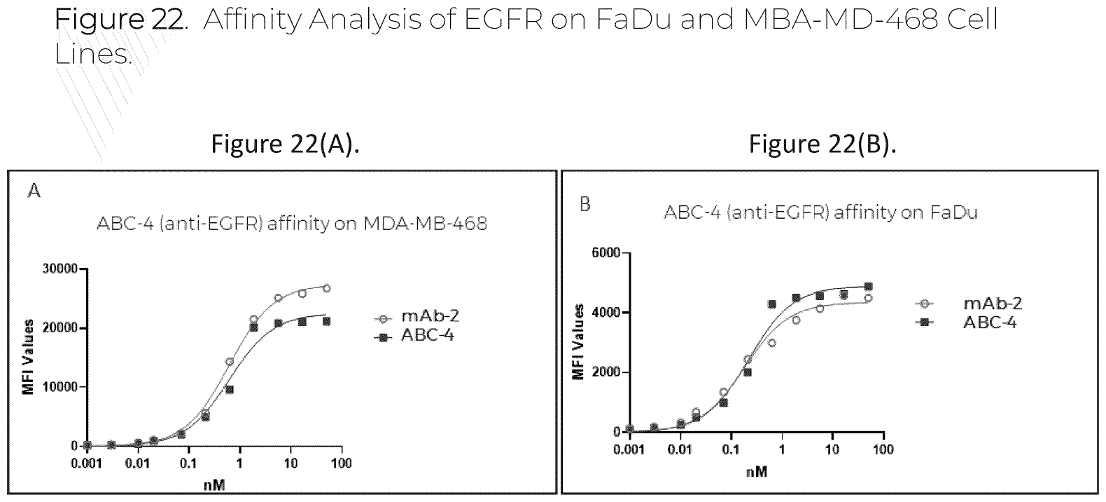

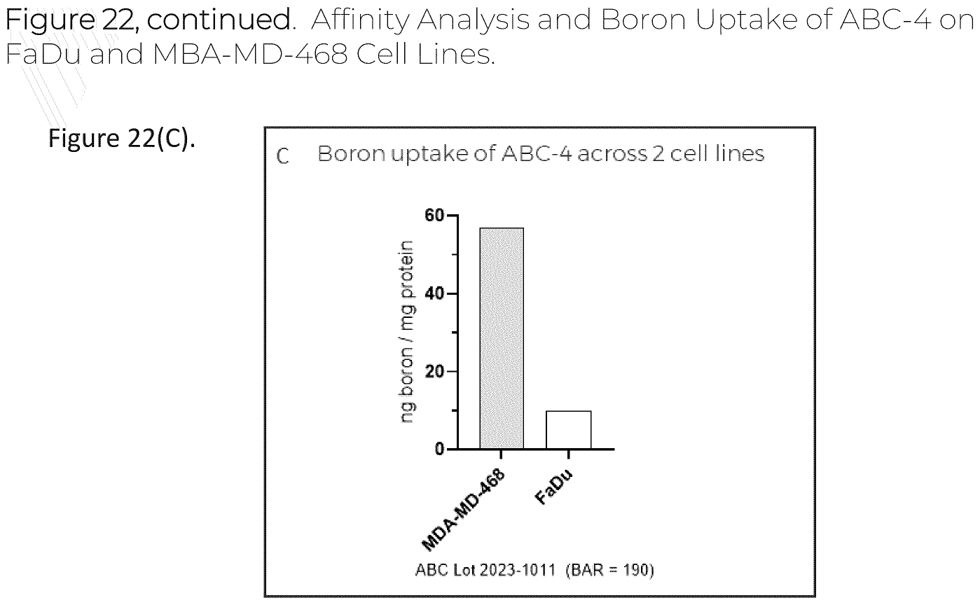

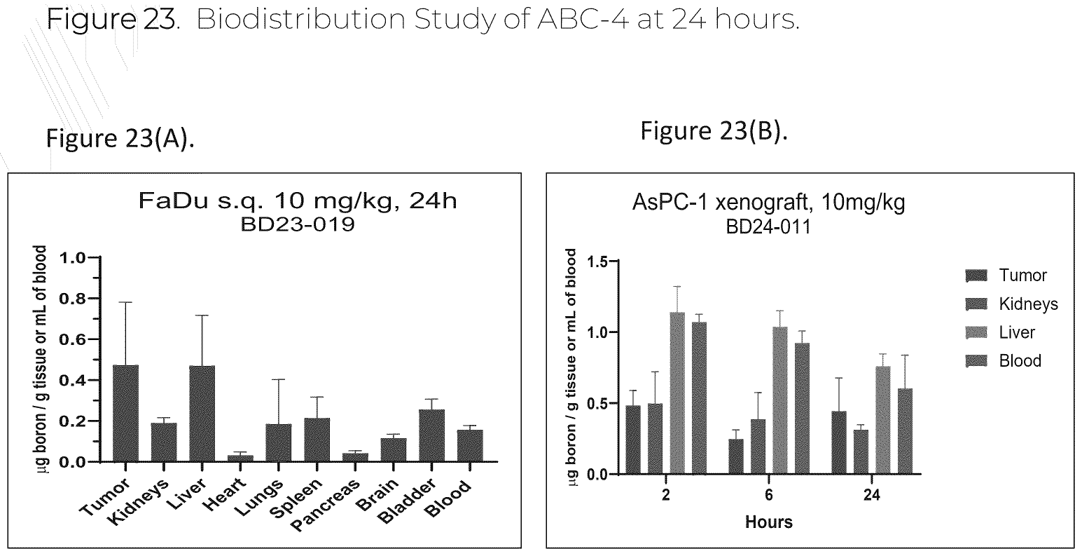

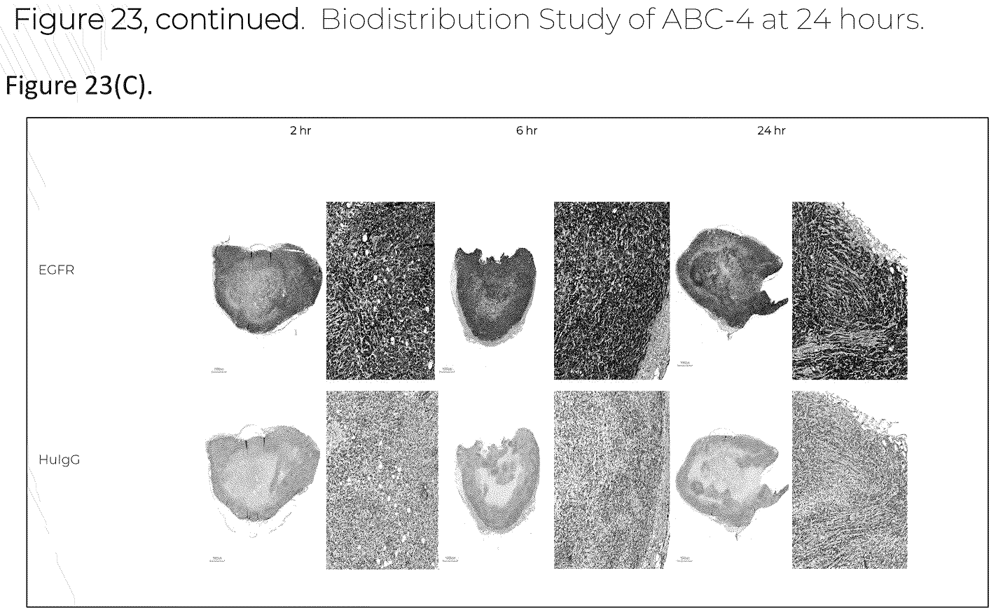

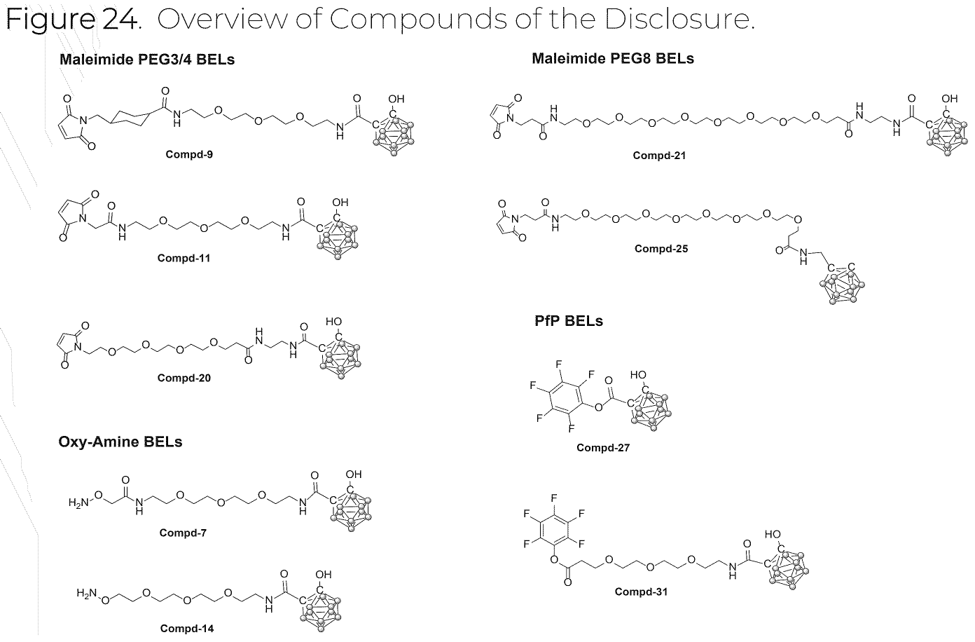

In the current disclosure, a poly-L-lysine conjugate is first activated on lysine side chains and modified to enable site-specific conjugation to mAb (See, FIG. 1). On the second step, the modified poly-L-lysine and mAb are ligated to afford a pre-ABC. The latter is purified by a mixed-mode chromatography such as ceramic hydroxyapatite. HMW aggregates are also removed during this step. The final purified product, penultimate ABC (i.e., a “pre-ABC”) is formulated and can be stockpiled. The last step taught in the current disclosure is conjugation to boron linkers (i.e., a BEL). The stoichiometric or near stoichiometric nature of the linking chemistry using either oxyamine- or maleimide-terminated BELs affords significant savings of the compounds composed of enriched-boron and also eliminates any subsequent purification conditions that would diminish the yield or otherwise lead to losses of the ultimate borylated conjugate.

### V.) Boron

### (a.) Boron Generally

Generally speaking, and for purposes of this disclosure, boron is a chemical element with symbol B and atomic number 5. Primarily used in chemical compounds, natural boron is composed of two stable isotopes, one of which is boron-10, and the other is boron-11. The boron-10 isotope is useful for capturing thermal neutrons, which makes it a promising tool in a therapeutic context using Boron Neutron Capture Therapy. Biologically, elemental boron, boron oxide, boric acid, boron-10, and boron-11 are relatively nontoxic to humans and animals (with toxicity similar to that of table salt). Based on the above-mentioned, it will be readily apparent to one of skill in the art that improved modalities for providing high concentrations of boron into a cancer cell are advantageous. It is an object of the present disclosure to provide that advantage.

### (b.) Boron Cluster(s)

Boron clusters or boranes are the name given to the class of synthetic hydrides of boron with a generic formula BₓH_{y}. The molecules of these compounds are electron deficient and so are highly reactive with respect to electron-pair donors. The boranes belong to a class of cluster compounds which have been the subject of developments in chemical bonding theory. For example, polyhedral skeletal electron pair theory (PSEPT), also referred to as Wade's rules provides electron counting rules useful for predicting the structure of clusters. Briefly, different rules (4n, 5n, or 6n) are invoked depending on the number of electrons per vertex. For example, the 4n rules are reasonably accurate in predicting the structures of clusters having about 4 electrons per vertex, as is the case for many boranes and carboranes. For such clusters, the structures are based on deltahedra, which are polyhedra in which every face is triangular. The 4n clusters are classified as closo-, nido-, arachno- or hypho-, based on whether they represent a complete (closo-) deltahedron, or a deltahedron that is missing one (nido-), two (arachno-) or three (hypho-) vertices. However, hypho clusters are relatively uncommon due to the fact that the electron count is high enough to fill anti-bonding orbitals and destabilize the 4n structure. If the electron count is close to 5 electrons per vertex, the structure often changes to one governed by the 5n rules, which are based on 3-connected polyhedra. As the electron count increases further, the structures of clusters with 5n electron counts become unstable, so the 6n rules can be implemented. The 6n clusters have structures that are based on rings. A molecular orbital treatment can be used to rationalize the bonding of cluster compounds of the 4n, 5n, and 6n types. In one embodiment the disclosure teaches the use of boron clusters in synthesizing a Boron Enriched Linker (“BEL”) comprising the boron clusters set forth in Table I. In a preferred embodiment, boron clusters of the disclosure comprise a 4n cluster. The polyhedral set forth in Table I are closo polyhedra, and are the basis for the 4n rules discussed, supra. The number of vertices in the cluster determines what polyhedron the structure is based on. In a further preferred embodiment, a boron cluster of the disclosure comprises a closo (4n+2) structure. In one embodiment of the present disclosure, the boron cluster is set forth in Table I(A).

### VI.) Carborane Based Boron Enriched PEG Linker Compounds (“BELs”)

It is an object of the present invention to provide carborane based boron enriched PEG linker compounds (“BEL” or “BELs” as the context necessitates) to be conjugated to an antibody or fragment thereof whereby the BEL comprises a plurality of modified boron clusters of the invention to create an antibody boron conjugate (ABC). As discussed, infra, ABCs of the present invention are used as a modality to provide concentrated amounts of boron to a cancer cell as a precursor for BNCT.

Generally speaking, a BEL of the disclosure may comprise several components including a linker attached to a boron containing cluster using a suitable linker attachment handle. For example, see BONDAREV, et. al, J. Am. Chem. Soc., 201(3) 13204-13211 (2013). The boron cluster is then used as a branching point for the addition of a plurality of boron clusters linked in a daisy chain fashion. The BELs of the present disclosure are designed with two (2) primary principles in mind. First, the BELs are designed to assess various modes of bio-conjugation known in the art, each of which is included within the scope of the present disclosure. Second, the BEL should endeavor to incorporate charged entities (e.g., lysine, primary amines, or quaternary amines) which shall render the BEL a zwitter-ion, thus effectively reducing the net negative charge.

In one embodiment, the BEL of the present invention comprises a linker of the following formula:

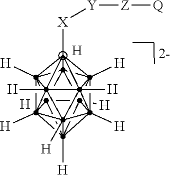

Whereby X is the functionalization at the vertex and is selected from the group consisting of NH₃, SH, or OH which creates a mono-substituted dodecaborates.

### Y=a Stretcher Unit;

Z=a Spacer Unit; and/or
Q=a bioconjugation handle

### (A) The Stretcher Unit

The Stretcher unit (Y), when present, is capable of linking an Antibody unit to a Spacer unit (—Z—), if present; or to a Boron cluster unit (-B). Useful functional groups that can be present on a mAb of the invention, either naturally or via chemical manipulation include, but are not limited to sulfhydryl, amino, hydroxyl, the anomerio hydroxyl group of a carbohydrate, and carboxyl. Suitable functional groups are sulfhydryl and amino acids. In one example, sulfhydryl groups can be generated by reduction of the intramolecular disulfide bonds of a mAb. In another embodiment, sulfhydryl groups can be generated by reaction of an amino group of a lysine moiety of a mAb with 2-iminothiolane (Traut's reagent) or other sulfhydryl generating reagents. In certain embodiments, the mAb is a recombinant antibody and is engineered to carry one or more lysines. In certain other embodiments, the recombinant mAb is engineered to carry additional sulfhydryl groups, e.g., additional cysteines. In one embodiment, the Stretcher unit forms a bond with a sulfur atom of the Antibody unit. The sulfur atom can be derived from a sulfhydryl group of an antibody.

In certain embodiments, the Stretcher unit is linked to the Antibody unit via a disulfide bond between a sulfur atom of the Antibody unit and a sulfur atom of the Stretcher unit.

In yet other embodiments, the Stretcher contains a reactive site that can form a bond with a primary or secondary amino group of an Antibody. Examples of these reactive sites include, but are not limited to, activated esters such as succinimide esters, 4 nitrophenyl esters, pentafluorophenyl esters, tetrafluorophenyl esters, anhydrides, acid chlorides, sulfonyl chlorides, isocyanates and isothiocyanates.

In some embodiments, the Stretcher contains a reactive site that is reactive to a modified carbohydrate's (—CHO) group that can be present on an Antibody. For example, a carbohydrate can be mildly oxidized using a reagent such as sodium periodate and the resulting (—CHO) unit of the oxidized carbohydrate can be condensed with a Stretcher that contains a functionality such as a hydrazide, an oxime, a primary or secondary amine, a hydrazine, a thiosemicarbazone, a hydrazine carboxylate, and an arylhydrazide such as those described by Kaneko et al., 1991, Bioconjugate Chem. 2:133-41, or quinolinone, such as those described by Qiang, 2013, JACS 135:4996-99

In a preferred embodiment, the Stretcher Unit is chosen from a carboxylic acid and its derivatives, alcohols, aliphatic carbon chains, and di-peptide or tri-peptide amino acids,

### (B) The Spacer Unit

The Spacer unit (—Z—), when present, links a Stretcher Unit to the Antibody unit when a Stretcher unit is present. Alternately, the Spacer unit links the Antibody unit to the boron cluster unit when the Stretcher unit is absent. Spacer units are of two general types: non-self-immolative or self-immolative. A non-self-immolative Spacer unit is one in which part, or all of the Spacer unit remains bound to the boron cluster moiety after cleavage, particularly enzymatic, of an antibody unit from the antibody boron conjugate. Examples of a non-self-immolative Spacer unit include but are not limited to a (glycine-glycine) Spacer unit and a glycine Spacer unit. When an ABC containing a glycine-glycine Spacer unit or a glycine Spacer unit undergoes enzymatic cleavage via an enzyme (e.g., a tumor-cell associated-protease, a cancer-cell-associated protease, or a lymphocyte-associated protease), a glycine-glycine-boron moiety or a glycine-boron moiety is cleaved from the antibody. In one embodiment, an independent hydrolysis reaction takes place within the target cell, cleaving the glycine-drug moiety bond and liberating the boron cluster.

In some embodiments, a non-self-immolative Spacer unit (—Y—) is -Gly-. In some embodiments, a non-self-immolative Spacer unit (—Y—) is -Gly-Gly.

In one embodiment, an ABC is provided in which the Spacer unit is absent (y=0), or a pharmaceutically acceptable salt or solvate thereof.

In some embodiments, —Y— is a p-aminobenzyl alcohol (PAB) unit whose phenylene portion is substituted with Qₘ wherein Q is —C₁-C₈ alkenyl, —C₁-C₈ alkynyl, —O—(C₁-C₈ alkyl), —O—(C₁-C₈ alkenyl), —O—(C₁-C₈alkynyl), -haolgen, -nitro or -cyano; and m is an integer ranging from 0-4. The alkyl, alkenyl and alkynyl groups, whether alone or as part of another group, can be optionally substituted.

In some embodiments, —Y— is a PAB group that is linked to -W_{w}- via the amino nitrogen atom of the PAB group, and connected directly to -D via a carbonate, carbamate or ether group as described by Toki et al., 2002, J. Org. Chem. 67:1866-1872.

Other examples of self-immolative spacers include, but are not limited to, aromatic compounds that are electronically similar to the PAB group such as 2-aminoimidazol-5-methanol derivatives (Hay et al., 1999, Bioorg. Med. Chem. Lett. 9:2237) and ortho or para-amin benzylacetals. Spacers can be used that undergo cyclization upon amide bond hydrolysis, such as substituted and unsubstituted 4-aminobutyric acid amides (Rodrigues et al., 1995, Chemistry Biology 2:223), appropriately substituted bicyclo[2.2.1] and bicyclo[2.2.2] ring systems (Storm et al., 1972, J. Amer. Chem. Soc. 94:5815) and 2-aminophenylpropionic acid amides (Amsberry et al., 1990, J. Org. Chem. 55:5867), Elimination of amine-containing drugs that are substituted at the α-position of glycine (Kingsbury et al., 1984, J. Med. Chem. 27:1447) are also examples of self-immolative spacers.

In a preferred embodiment, the Spacer Unit is selected from the group consisting of an aliphatic chain (preferred range from C3-C8), a fatty acid, or a PEG (ranging from 3-12).

### (C) The Bio-Conjugation Handle

The bioconjugation handle (-Q-) of the present invention comprises phenyl isothiocyanate, malemide, succinamide, dibromomaleimide, dithiophenolmaleimide, azide, propargyl, dibenzocyclooctyne or oxyamine and allows for the conjugation of the BEL to a mAb of the invention or to additional BELs.

### (D) Boron Antibody Ratio (BAR)

The disclosure provides a new modality for providing boron to a cell for the use of BNCT via an Antibody Boron Conjugate (ABC). An ABC of the present disclosure furthers traditional conjugation chemistry paradigms by allowing significant amounts of boron to be concentrated into a cell. This is the crucial first step needed to enhance common BNCT treatment paradigms. In order to measure the amount of boron in an ABC relative to the antibody to which it is conjugated, the inventors utilized a ratio known as the Boron-Antibody Ratio (BAR). Briefly, the BAR represents the average number of boron clusters conjugated to a particular antibody. The BAR represents an important attribute to an ABC. For example, the BAR value affects the stability of the mAb as well as the overall stability of the ABC. In order to enhance the efficacy of BNCT, the ability to deliver a high concentration of boron to a cancer cell is advantageous.

In one aspect of the present disclosure, a BAR of 12 to 60 is within the scope of the invention. In another embodiment, a BAR of 144-720 is within the scope of the invention. In one embodiment an ABC with a BAR from about 2-1000 is within the scope of the present invention.

### (i) Lo-BAR

Conjugates that carry up to 100 boron atoms and are expected to deliver the amount of boron that is insufficient for the efficient neutron capture and may not lead to cancer cell death.

### (ii) Hi-BAR

Conjugates that carry greater than 100 of boron atoms and are expected to deliver the amount of boron beyond of what is minimally required for the efficient neutron capture and will lead to cancer cell death.

### (E) Markush Formula (I) and Formula II

In one embodiment, a BEL with the following formula is within the scope of the present disclosure:

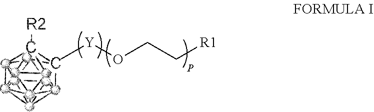

Wherein,
R1=—COCH₂-maleimide, —COCH₂CH₂-maleimide, —COCH₂-aminooxy, —COCH₂CH₂-aminooxy, -1-CO-cyclohexyl-4-CH₂-maleimide, -pentafluorophenyl, —CO₂-pentafluorophenyl;
R2=—H, —OH, —OAc, -Glucamine;
Y=—CO₂—, —CONH—CH₂CH₂—, —CONH—CH₂CH₂—NHCO—CH₂CH₂—, —CH₂NHCO—CH₂CH₂—;
P=polyethylene glycol units of n=0, 1, 2, 3, 4, 5, 6, 7, or 8; and in some embodiments the compound is a single enantiomer at C4 as the R stereocenter or is a single enantiomer at C4 as the S stereocenter.

In another embodiment, a BEL with the following formula is within the scope of the present disclosure:

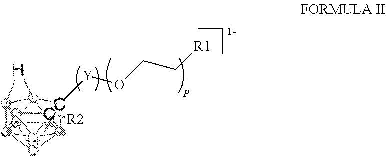

Wherein,
R1=—COCH₂-maleimide, —COCH₂CH₂-maleimide, —COCH₂-aminooxy, —COCH₂CH₂-aminooxy, -1-CO-cyclohexyl-4-CH₂-maleimide, -pentafluorophenyl, —CO₂-pentafluorophenyl;

### R2=—H, —OH, —OAc, -Glucamine;

Y=—CO₂—, —CONH—CH₂CH₂—, —CONH—CH₂CH₂—NHCO—CH₂CH₂—, —CH₂NHCO—CH₂CH₂—;
P is polyethylene glycol units of n=0, 1, 2, 3, 4, 5, 6, 7, or 8; and in some embodiments the compound is a single enantiomer at C4 as the R stereocenter or is a single enantiomer at C4 as the S stereocenter.

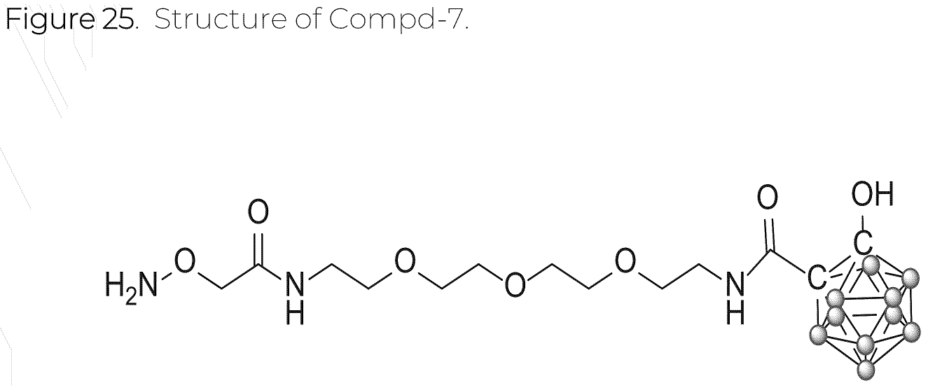

From the aforementioned (Formula I and II) and using the conceptual schema for Carborane Based Boron Enriched PEG Linker Compounds set forth in FIGS. 25-33, the following Carborane Based Boron Enriched PEG Linker Compounds are within the scope of the disclosure.

### (F) Maleimide PEG ¾ Linker Compounds

In one embodiment, a BEL with the following formula is within the scope of the present disclosure:

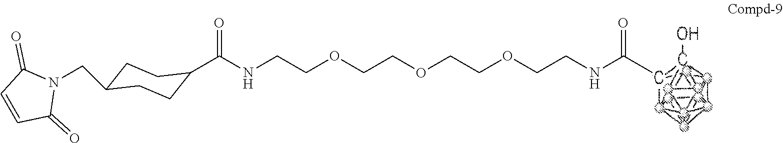 [SMILES: CC(=O)NCCOCCOCCOCCNC(=O)C1CCC(CN2C(=O)C=CC2=O)CC1]

In another embodiment, a BEL with the following formula is within the scope of the present disclosure:

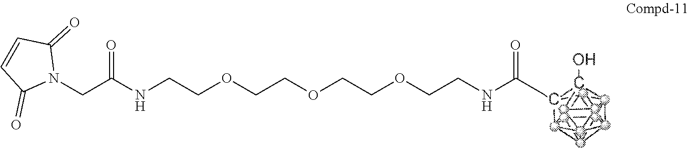 [SMILES: CC(=O)NCCOCCOCCOCCNC(=O)CN1C(=O)C=CC1=O]

In another embodiment, a BEL with the following formula is within the scope of the present disclosure:

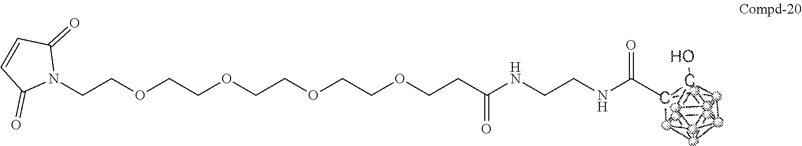 [SMILES: CC(=O)NCCNC(=O)CCOCCOCCOCCOCCN1C(=O)C=CC1=O]

It will be appreciated by one of ordinary skill in the art that modifications to Compd-9, Compd-11, and Compd-20 may be made while keeping the same functional integrity. For example, as will be appreciated by one of ordinary skill in the art, the introduction of certain modifications will pave the way for substantially higher BAR.

### (G) Oxy-Amine PEG3 Linker Compounds

In one embodiment, a BEL with the following formula is within the scope of the present disclosure:

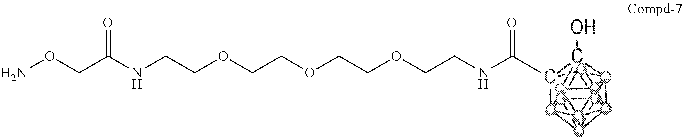 [SMILES: CC(=O)NCCOCCOCCOCCNC(=O)CON]

In another embodiment, a BEL with the following formula is within the scope of the present disclosure:

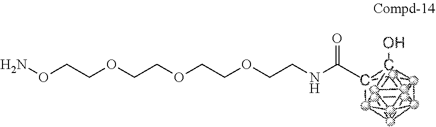 [SMILES: CC(=O)NCCOCCOCCOCCON]

It will be appreciated by one of ordinary skill in the art that modifications to Compd-7 and Compd-14 may be made while keeping the same functional integrity. For example, as will be appreciated by one of ordinary skill in the art, the introduction of certain modifications will pave the way for substantially higher BAR.

### (H) Maleimide PEG₈ Linker Compounds

In one embodiment, a BEL with the following formula is within the scope of the of the present disclosure:

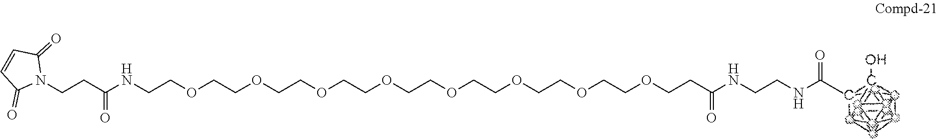 [SMILES: CC(=O)NCCNC(=O)CCOCCOCCOCCOCCOCCOCCOCCOCCNC(=O)CCN1C(=O)C=CC1=O]

In another embodiment, a BEL with the following formula is within the scope of the of the present disclosure:

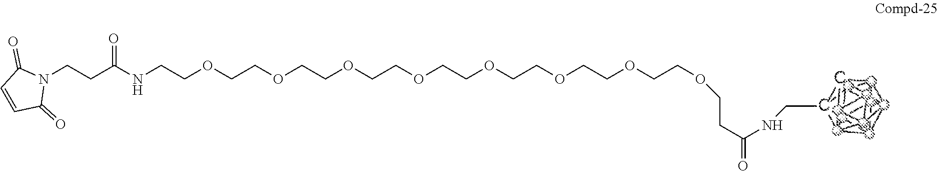 [SMILES: CCNC(=O)CCOCCOCCOCCOCCOCCOCCOCCOCCNC(=O)CCN1C(=O)C=CC1=O]

It will be appreciated by one of ordinary skill in the art that modifications to Compd-21 and Compd-25 may be made while keeping the same functional integrity. For example, as will be appreciated by one of ordinary skill in the art, the introduction of certain modifications will pave the way for substantially higher BAR.

### (I) Maleimide PEG₈ Linker Compounds

In one embodiment, a BEL with the following formula is within the scope of the present disclosure:

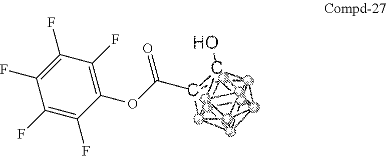

In another embodiment, a BEL with the following formula is within the scope of the present disclosure:

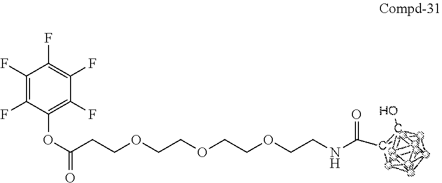 [SMILES: CC(=O)NCCOCCOCCOCCC(=O)Oc1c(F)c(F)c(F)c(F)c1F]

It will be appreciated by one of ordinary skill in the art that modifications to Compd-27 and Compd-31 may be made while keeping the same functional integrity. For example, as will be appreciated by one of ordinary skill in the art, the introduction of certain modifications will pave the way for substantially higher BAR.

### VII.) Boron Neutron Capture Therapy Using BELs

One aspect of the present disclosure is the use of BELs and ABCs as a modality for Boron Neutron Capture Therapy (BNCT). Briefly, BNCT is a binary treatment modality in which neither component alone is lethal nor highly toxic. The two components comprise (i) the infusion or delivery of a capture compound, which preferentially is concentrated in the tumor, and (ii) the irradiation of the tumor site by neutrons. Given the large cross-section of thermal neutron interactions with ¹⁰B, there is consequently a high probability of splitting a boron nucleus into He and Li. Given that the ionization capability of He and Li is high, and the runs are short, then the cells preferably enriched by boron are killed and the healthy cells are damaged much less. Given this, the advantage of BNCT is the destruction of tumor cells without a highly traumatic surgical procedure. However, as will be understood by one of skill in the art, success is predicated on the concentration and selective localization of ¹⁰B in tumor cells.

In one embodiment, ¹⁰B is concentrated into a BEL of the invention and is conjugated to an antibody or fragment thereof to create an ABC. The ABC is then given to a patient, and the BEL is localized into a tumor cell. The ¹⁰B-enriched ABC is selectively concentrated in the tumor, which is then irradiated with epithermal neutrons, leading to the selective destruction of tumor cells.

### VIII. Methods of Delivering BELs to a Cell

As will be appreciated by one of ordinary skill in the art, the ability to efficiently deliver high concentrations of boron to a cell is an advantage of the present invention.

It is shown that the BELs of the present disclosure enables a higher amount of boron to be administered to a cell safely in mammals. Briefly, BELs of the disclosure are prepared as set forth in the disclosure. The BAR is pre-determined, and the synthesis is set forth accordingly. The BEL is conjugated to an mAb of the invention using methods known in the art. The resulting ABC targets the antigen or antigen binding fragment, and the boron is concentrated into a cancer cell.

Accordingly, it is shown that ABCs of the disclosure loaded with boron can be modified to selectively localize in high concentrations in tumor cells.

### IX.) Kits/Articles of Manufacture

For use in the laboratory, prognostic, prophylactic, diagnostic and therapeutic applications described herein, kits are within the scope of the invention. Such kits can comprise a carrier, package, or container that is compartmentalized to receive one or more containers such as vials, tubes, and the like, each of the container(s) comprising one of the separate elements to be used in the method, along with a label or insert comprising instructions for use, such as a use described herein. For example, the container(s) can comprise an ABC that is or can be detectably labeled and/or is loaded with a BEL of the disclosure. Kits can comprise a container comprising a drug unit. The kit can include all or part of the ABCs and/or BELs.

The kit of the invention will typically comprise the container described above and one or more other containers associated therewith that comprise materials desirable from a commercial and user standpoint, including buffers, diluents, filters, needles, syringes; carrier, package, container, vial and/or tube labels listing contents and/or instructions for use, and package inserts with instructions for use.

A label can be present on or with the container to indicate that the composition is used for a specific therapy or non-therapeutic application, such as a prognostic, prophylactic, diagnostic or laboratory application, and can also indicate directions for either in vivo or in vitro use, such as those described herein. Directions and/or other information can also be included on an insert(s) or label(s) which is included with or on the kit. The label can be on or associated with the container. A label can be on a container when letters, numbers or other characters forming the label are molded or etched into the container itself; a label can be associated with a container when it is present within a receptacle or carrier that also holds the container, e.g., as a package insert. The label can indicate that the composition is used for diagnosing, treating, prophylaxing or prognosing a condition, such as a cancer or other immunological disorder.

The terms “kit” and “article of manufacture” can be used as synonyms,

In another embodiment of the invention, an article(s) of manufacture containing compositions, such as ABCs and/or BELs and/or ABCs loaded with BELs. The article of manufacture typically comprises at least one container and at least one label. Suitable containers include, for example, bottles, vials, syringes, and test tubes. The containers can be formed from a variety of materials such as glass, metal, or plastic. The container can hold ABCs and/or BELs.

The container can alternatively hold a composition that is effective for treating, diagnosis, prognosing or prophylaxing a condition and can have a sterile access port (for example the container can be an intravenous solution bag or a vial having a stopper pierceable by a hypodermic injection needle). The active agents in the composition can be an ABC loaded with a BEL.

The article of manufacture can further comprise a second container comprising a pharmaceutically acceptable buffer, such as phosphate-buffered saline, Ringer's solution, and/or dextrose solution. It can further include other materials desirable from a commercial and user standpoint, including other buffers, diluents, filters, stirrers, needles, syringes, and/or package inserts with indications and/or instructions for use.

### EXEMPLARY EMBODIMENTS

1) A compound comprising a chemical structure as follows:

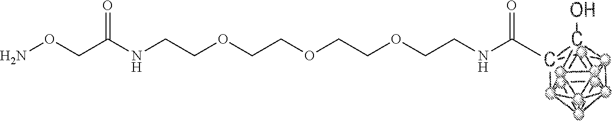 [SMILES: CC(=O)NCCOCCOCCOCCNC(=O)CON]

2) The compound of claim 1, wherein the composition is conjugated to an antibody.
3) A method of producing a composition of claim 1.
4) A kit comprising the composition of claim 1.
5) A compound comprising a chemical structure as follows:

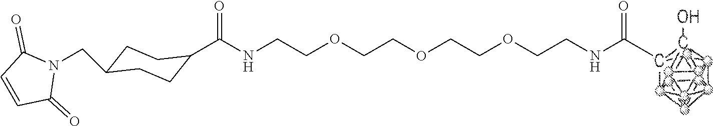 [SMILES: CC(=O)NCCOCCOCCOCCNC(=O)C1CCC(CN2C(=O)C=CC2=O)CC1]

6) The compound of claim 5, wherein the composition is conjugated to an antibody.
7) A method of producing a composition of claim 5.
8) A kit comprising the composition of claim 5.
9) A compound comprising a chemical structure as follows:

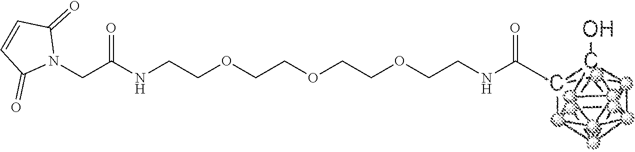 [SMILES: CC(=O)NCCOCCOCCOCCNC(=O)CN1C(=O)C=CC1=O]

10) The compound of claim 9, wherein the composition is conjugated to an antibody.
11) A method of producing a composition of claim 9.
12) A kit comprising the composition of claim 9.
13) A compound comprising a chemical structure as follows:

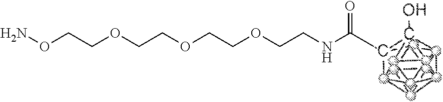 [SMILES: CC(=O)NCCOCCOCCOCCON]

14) The compound of claim 13, wherein the composition is conjugated to an antibody.
15) A method of producing a composition of claim 13.
16) A kit comprising the composition of claim 13.
17) A compound comprising a chemical structure as follows:

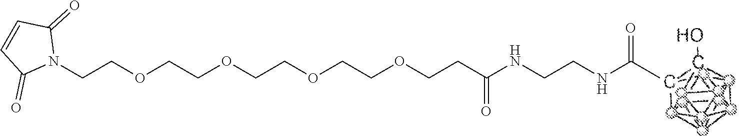 [SMILES: CC(=O)NCCNC(=O)CCOCCOCCOCCOCCN1C(=O)C=CC1=O]

18) The compound of claim 17, wherein the composition is conjugated to an antibody.
19) A method of producing a composition of claim 17.
20) A kit comprising the composition of claim 17.
21) A compound comprising a chemical structure as follows:

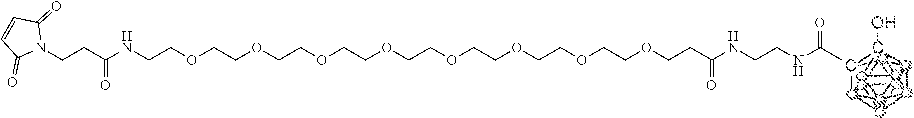 [SMILES: CC(=O)NCCNC(=O)CCOCCOCCOCCOCCOCCOCCOCCOCCNC(=O)CCN1C(=O)C=CC1=O]

22) The compound of claim 21, wherein the composition is conjugated to an antibody.
23) A method of producing a composition of claim 21.
24) A kit comprising the composition of claim 21.
25) A compound comprising a chemical structure as follows:

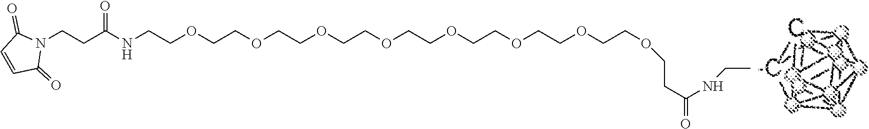 [SMILES: CCNC(=O)CCOCCOCCOCCOCCOCCOCCOCCOCCNC(=O)CCN1C(=O)C=CC1=O]

26) The compound of claim 25, wherein the composition is conjugated to an antibody.
27) A method of producing a composition of claim 25.
28) A kit comprising the composition of claim 25.
29) A compound comprising a chemical structure as follows:

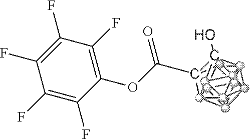

30) The compound of claim 29, wherein the composition is conjugated to an antibody.
31) A method of producing a composition of claim 29.
32) A kit comprising the composition of claim 29.
33) A compound comprising a chemical structure as follows:

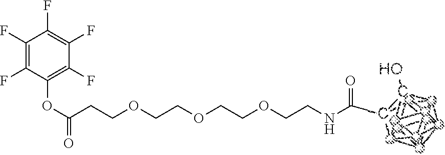 [SMILES: CC(=O)NCCOCCOCCOCCC(=O)Oc1c(F)c(F)c(F)c(F)c1F]

34) The compound of claim 33, wherein the composition is conjugated to an antibody.
35) A method of producing a composition of claim 33.
36) A kit comprising the composition of claim 33.
37) An Antibody Boron Conjugate (ABC) comprising,
   a. an antibody; and
   b. a carborane-based boron enriched PEG linker (BEL), wherein said BEL is conjugated to said antibody.
38) The ABC of claim 37, wherein the BEL comprises a chemical structure essentially as follows:

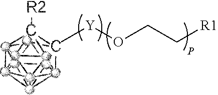

wherein,
R1=—COCH₂-maleimide, —COCH₂CH₂-maleimide, —COCH₂-aminooxy, —COCH₂CH₂-aminooxy, -1-CO-cyclohexyl-4-CH₂-maleimide, -pentafluorophenyl, —CO₂-pentafluorophenyl;
R2=—H, —OH, —OAc, -Glucamine;
Y=—CO₂—, —CONH—CH₂CH₂—, —CONH—CH—CH₂—NHCO—CH₂CH₂—, —CH₂NHCO—CH₂CH₂—;
P=polyethylene glycol units of n=0, 1, 2, 3, 4, 5, 6, 7, or 8; and in some embodiments the compound is a single enantiomer at C4 as the R stereocenter or is a single enantiomer at C4 as the S stereocenter.
39) The ABC of claim 38, wherein the boron antibody ratio (BAR) is from 12-280.
40) The ABC of claim 38, further comprising a stretcher unit.
41) The ABC of claim 38, further comprising a spacer unit.
42) The ABC of claim 38, further comprising a bio-conjugation handle.
43) The ABC of claim 38, wherein the antibody binds to EGFR.
44) The ABC of claim 38, wherein the antibody binds to HER2/neu (ErbB2).
45) A method of producing the ABC of claim 38.
46) A kit comprising the ABC of claim 38.
47) The ABC of claim 37, wherein the BEL comprises a chemical structure essentially as follows:

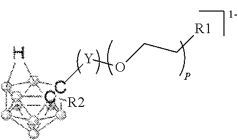

wherein,
R1=—COCH₂-maleimide, —COCH₂CH₂-maleimide, —COCH₂-aminooxy, —COCH₂CH₂-aminooxy, -1-CO-cyclohexyl-4-CH₂-maleimide, -pentafluorophenyl, —CO₂-pentafluorophenyl;
R2=—H, —OH, —OAc, -Glucamine;
Y=—CO₂—, —CONH—CH—CH₂—, —CONH—CH₂CH₂—NHCO—CH₂CH₂—, —CH₂NHCO—CH—CH₂;
P is polyethylene glycol units of n=0, 1, 2, 3, 4, 5, 6, 7, or 8; and
in some embodiments the compound is a single enantiomer at C4 as the R stereocenter or is a single enantiomer at C4 as the S stereocenter.
48) The ABC of claim 47, wherein the boron antibody ratio (BAR) is from 12-280.
49) The ABC of claim 47, further comprising a stretcher unit.
50) The ABC of claim 47, further comprising a spacer unit.
51) The ABC of claim 47, further comprising a bio-conjugation handle.
52) The ABC of claim 47, wherein the antibody binds to EGFR.
53) The ABC of claim 47, wherein the antibody binds to HER2/neu (ErbB2).
54) A method of producing the ABC of claim 47.
55) A kit comprising the ABC of claim 47.
56) A method of performing Neutron Capture Therapy in the treatment of human cancer comprising:
   a. conjugating a composition of any of claims 1, 5, 9, 13, 17, 21, 25, 29, and 33 with an antibody to create an antibody boron conjugate ABC;
   b. injecting the ABC into a tumor, whereby said composition accumulates into a tumor cell; and
   c. irradiating the accumulated composition with neutrons.
57) The method of claim 56, wherein the irradiation comprises epithermal neutrons.
58) The method of claim 56, wherein the irradiation triggers neutron activation.
59) The method of claim 56, wherein the antibody binds EGFR.
60) The methods of claim 56, wherein the antibody binds HER2/neu.
61) The method of claim 56, wherein the cancer is selected from the group consisting of breast cancer, brain cancer, gastric cancer, lung cancer, colon cancer, and head and neck cancer.
62) A method of producing an antibody-drug-conjugate (ABC) by the process comprising,
   a. activation of a lysine side chain(s) and modification to enable site-specific conjugation to a mAb;
   b. ligation of the modified poly-L-lysine and mAb to create a pre-ABC;
   c. purifying the pre-ABC by a mixed-mode chromatography, whereby the mixed-mode chromatography comprises ceramic hydroxyapatite;
   d. removal of HMW aggregates;
   e. conjugation of said pre-ABC to a boron enriched linker
63) The method of claim 62, wherein the BEL is a carborane-based boron enriched PEG linker.
64) The method of claim 62, wherein the BEL utilizes oxyamine terminated linking chemistry.
65) The method of claim 62, wherein the BEL utilizes maleimide terminated linking chemistry
66) The method of claim 62, wherein the mAb binds to EGFR.
67) The method of claim 62, wherein the mAb binds to Her2
68) The method of claim 62 consisting essentially of the of the steps shown in FIG. 1.

### EXAMPLES

Various aspects of the invention are further described and illustrated by way of the several examples that follow, none of which is intended to limit the scope of the invention.

### Example 1: Synthesis of Compd-7. Compd-9, and Compd-11 Intermediates.

The synthesis of Compd-7, Compd-9, and Compd-11 was performed using the following Scheme:

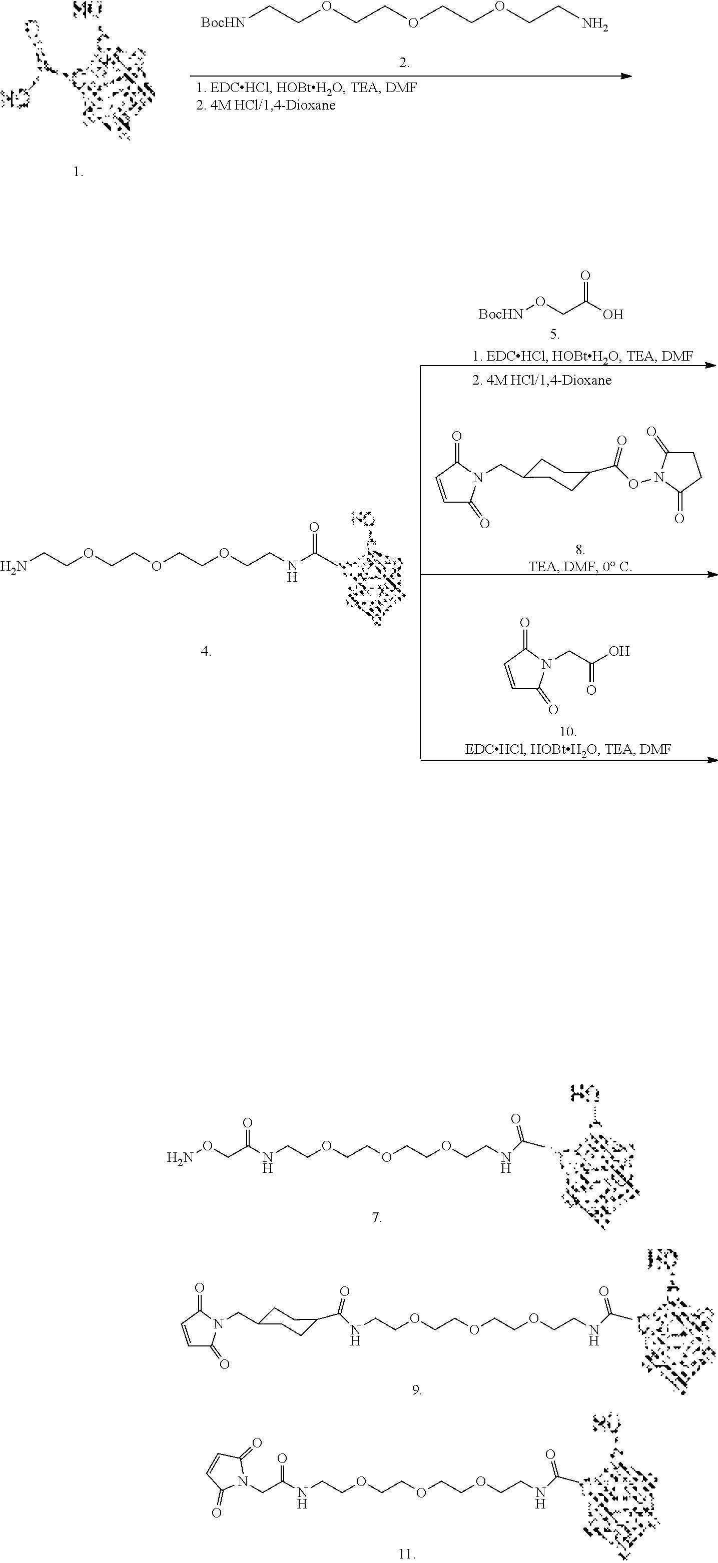

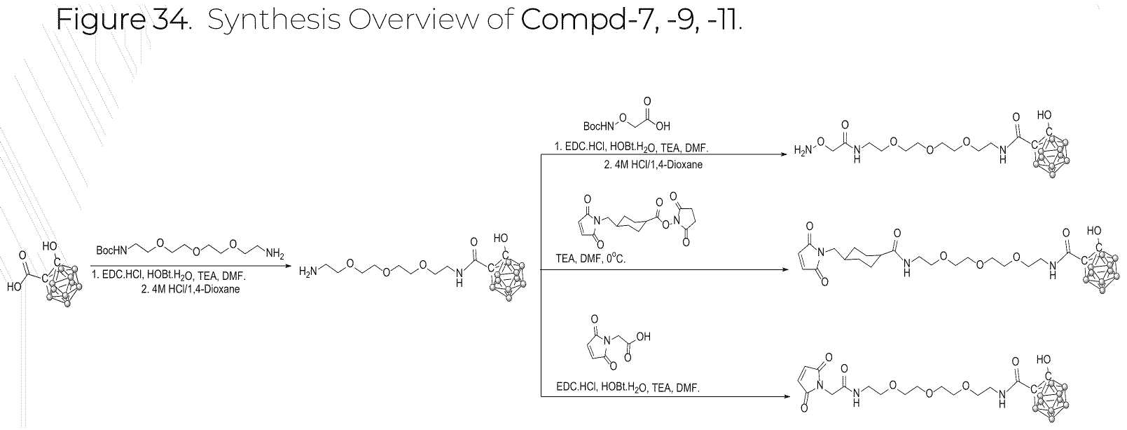

Briefly, amide coupling of Salborin (1.) and PEG4-amine (2.) with EDC·HCl, HOBt·H₂O and TEA in anhydrous DMF solvent afforded carbamate-PEG4-salborin (3.) intermediate. Boc deprotection of intermediate 3. with 4M HCl/1,4-dioxane gave the general intermediate amine-PEGA-salborin (4.) which was coupled to Boc-amino-oxy (5.) to obtain intermediate 6. Boc-removal of intermediate 6. was done with 4M HCl/1,4-dioxane to obtain aminooxy-PEGA-salborin (7.) using standard methods. Concurrently, amine intermediate 4. coupled to SMCC (8.) in the presence of TEA in anhydrous DMF solvent at zero degrees afforded target product MCC-PEG4-salborin (9.). Finally, amine intermediate 4. coupled to compound 10. gave target product mal-amido-PEG-salborin (11). (See, FIG. 34).

The synthesis of tert-butyl (1-oxo-1-salborin-5,8,11-trioxa-2-azatridecan-13-yl)carbamate (3.) was performed in the following manner. In a round bottom flask charged with a stir bar, a mixture of tert-butyl (2-(2-(2-(2-aminoethoxy)ethoxy)ethoxy)ethyl)carbamate (2.) (1.1 eq, 1 mmol), salborin (1.) (1.0 eq, 1 mmol), EDC·HCl (1.1 eq, 1 mmol), HOBt·H₂O (1.1 eq, 1 mmol) and TEA (1.1 eq, 1 mmol) was dissolved in anhydrous DMF (1 mL/mmol). The reaction mixture was kept overnight and concurrently monitored by LC/MS. Upon completion, the reaction mixture was diluted with brine (5 mL) and extracted with ethyl acetate (5 mL×3). The organic layer was dried over sodium sulfate and removed under reduced pressure in vacuo. The crude mixture was subjected to prep-LC (10 to 80% ACN/H₂O) resulting in tert-butyl (1-oxo-1-salborin-5,8,11-trioxa-2-azatridecan-13-yl) carbamate (3.) as a clear solid having the following chemical structure:

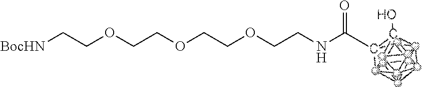 [SMILES: CC(=O)NCCOCCOCCOCCNC(=O)OC(C)(C)C]

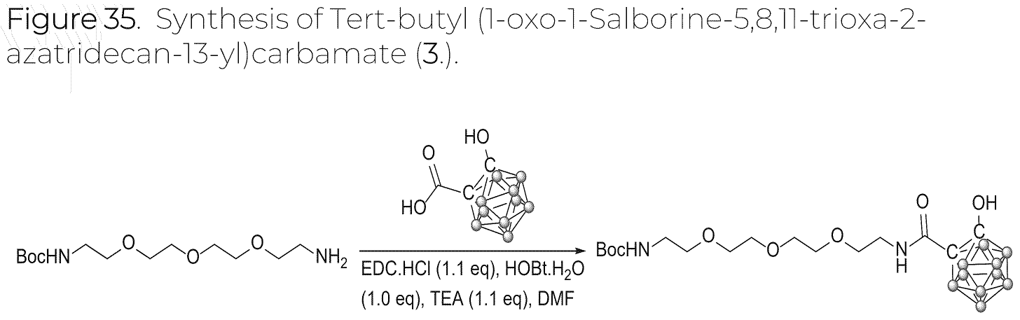

### (See, FIG. 35).

The synthesis of N-(2-(2-(2-(2-aminoethoxy)ethoxy)ethoxy)ethyl) salbamide (4.) was performed in the following manner. In a flame-dried round bottom flask charged with a stir bar, tert-butyl (1-oxo-1-salborin-5,8,11-trioxa-2-azatridecan-13-yl)carbamate (3.) was dissolved in excess 4M HCl in 1,4-dioxane. The reaction was monitored by LC/MS. Upon completion, it was concentrated under reduced pressure to obtain N-(2-(2-(2-(2-aminoethoxy)ethoxy)ethoxy)ethyl) salbamide (4.) as a pure, translucent semi liquid having the following chemical structure:

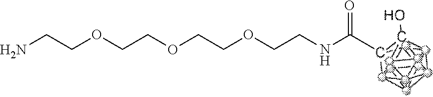 [SMILES: CC(=O)NCCOCCOCCOCCN]

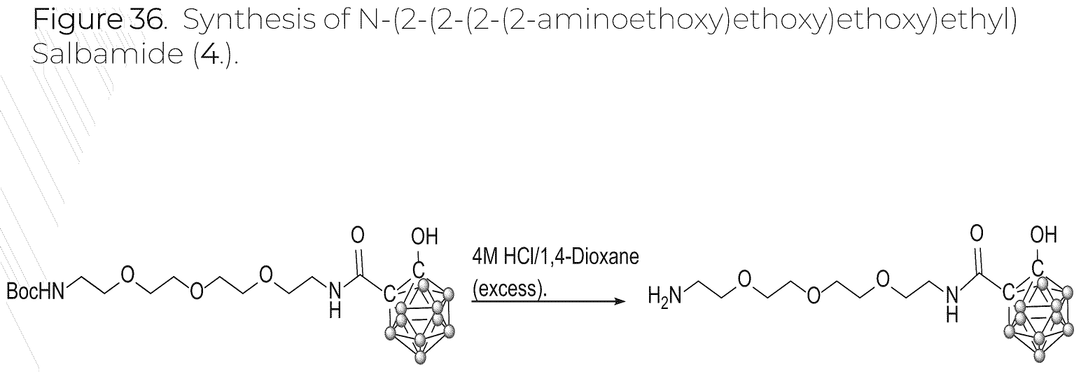

### (See, FIG. 36).

The synthesis of tert-butyl ((1,15-dioxo-1-Salborine-5,8,11-trioxa-2,14-diazahexadecan-16-yl)oxy)carbamate (6.) was performed in the following manner. In a round bottom flask charged with a stir bar, a mixture of N-(2-(2-(2-(2-aminoethoxy)ethoxy)ethoxy)ethyl) salbamide (4.) (1.1 eq, 1 mmol), 2-((tert-butoxycarbonyl)amino)oxy)acetic acid (5.) (1.0 eq, 1 mmol), EDC-HCl (1.1 eq, 1 mmol), HOBt-H₂O (1.1 eq, 1 mmol) and TEA (1.1 eq, 1 mmol) was dissolved in anhydrous DMF (1 mL/mmol). The reaction mixture was kept overnight and concurrently monitored by LC/MS. On completion, the reaction mixture was diluted with brine (5 ml) and extracted with ethyl acetate (5 ml×3). The organic layer was dried over sodium sulfate and removed under reduced pressure. The crude mixture was subjected to prep-LC (10 to 80% ACN/H₂O) resulting in tert-butyl ((1,15-dioxo-1-salborin-5,8,11-trioxa-2,14-diazahexadecan-16-yl)oxy)carbamate (6.) as a clear solid having the following chemical structure:

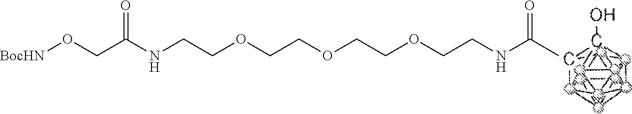 [SMILES: CC(=O)NCCOCCOCCOCCNC(=O)CONC(=O)OC(C)(C)C]

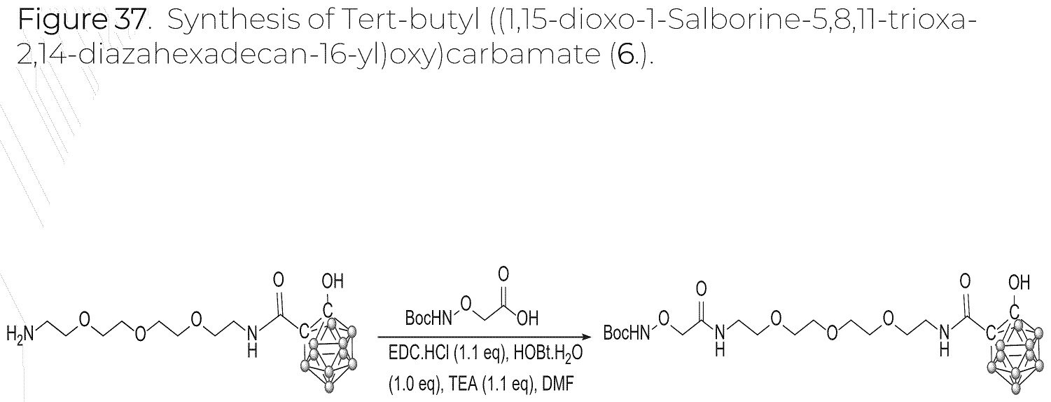

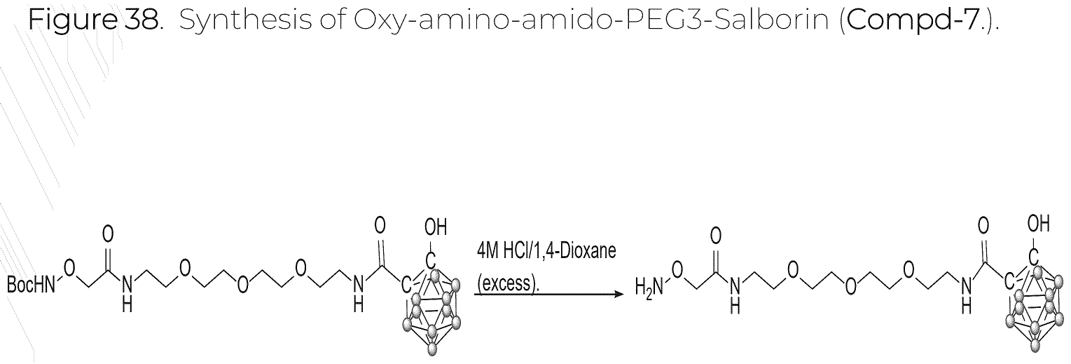

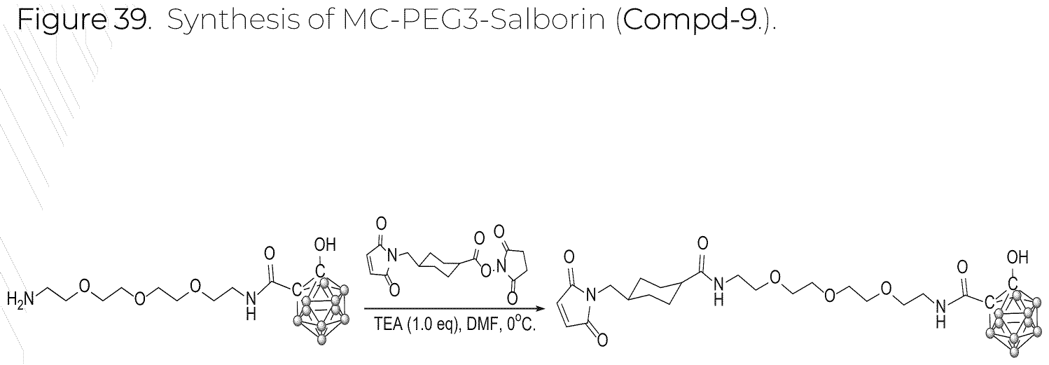

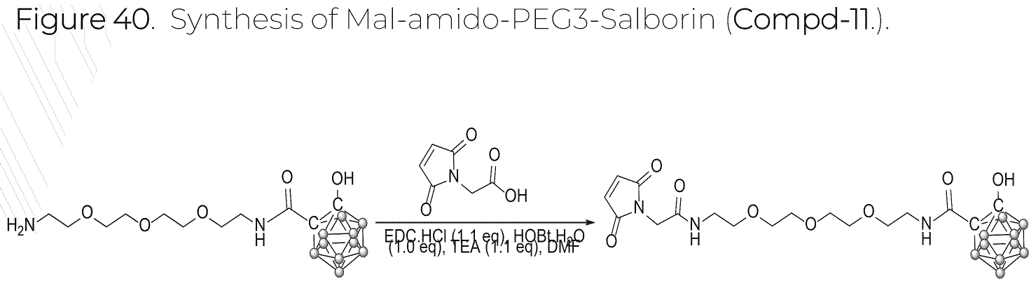

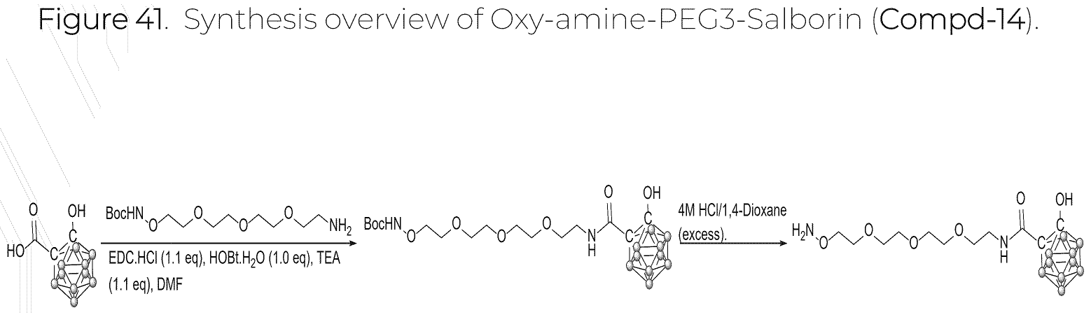

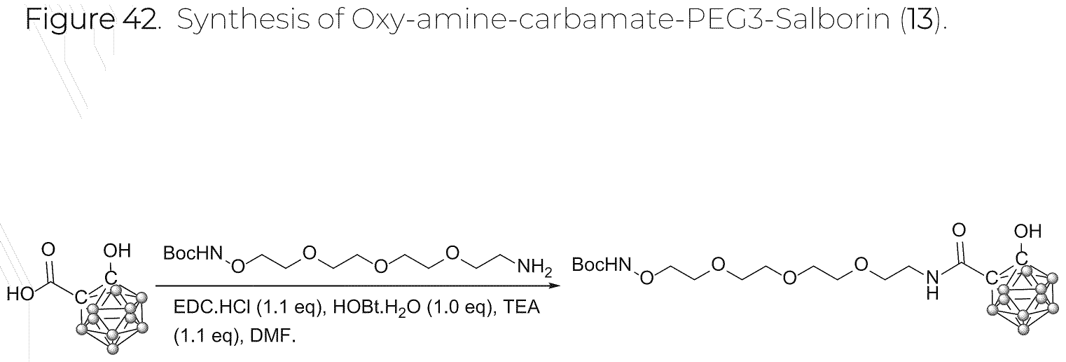

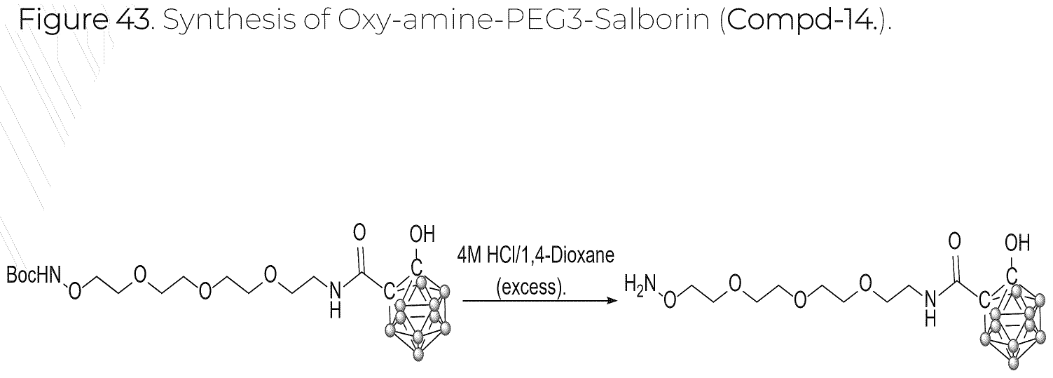

### (See, FIG. 37).

### Example 2: Synthesis of aminooxy-amido-PEG.-salborin-Compd-7.

Using the general scheme set forth in Example 1, Compd-7 was synthesized in the following manner. Briefly, in a flame-dried round bottom flask charged with a stir bar, tert-butyl((1,15-dioxo-1-salborin-5,8,11-trioxa-2,14-diazahexadecan-16-yl)oxy)carbamate (6.) was dissolved in excess 4M HCl in 1,4-dioxane. The reaction was monitored with LC/MS. On completion it was concentrated under reduced pressure. The crude mixture was subjected to prep-LC (10 to 80% ACN/H₂O) resulting in aminooxy-amido-PEG₃-salborin (7.) as a clear oil.

The resulting synthesis provided Compd-7 which contains the following chemical structure:

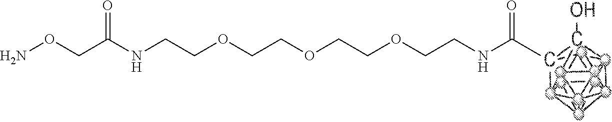 [SMILES: CC(=O)NCCOCCOCCOCCNC(=O)CON]

### (See, FIG. 25).

### Example 3: Synthesis of MC-PEG₃-salborin-Compd-9.

Using the general scheme set forth in Example 1, Compd-9 was synthesized in the following manner. Briefly, in a round bottom flask charged with a stir bar, a mixture of N-(2-(2-(2-(2-aminoethoxy)ethoxy)ethoxy)ethyl) salbamide (4.) (1.1 eq, 1 mmol), 2,5-dioxopyrrolidin-1-yl 4-((2,5-dioxo-2,5-dihydro-1H-pyrrol-1-yl)methyl)cyclohexane-1- carboxylate (8.) (1.0 eq, 1 mmol) and TEA (1.1 eq, 1 mmol) was dissolved in anhydrous DMF (1 mL/mmol) at zero degrees. The reaction mixture was kept and concurrently monitored by LC/MS. On completion, the reaction mixture was diluted with distilled H₂O (2 mL) and extracted with ethyl acetate (3 mL×3). The organic layer was dried over sodium sulfate and removed under reduced pressure. The crude mixture was subjected to prep-LC (10 to 80% ACN/H₂O) resulting in MC-PEG₃-salborin (9.) as an oil.

The resulting synthesis provided Compd-9 having the following chemical structure:

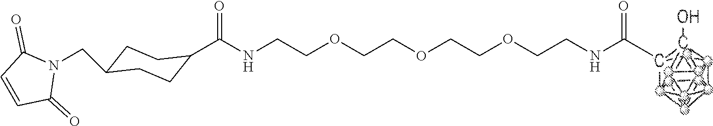 [SMILES: CC(=O)NCCOCCOCCOCCNC(=O)C1CCC(CN2C(=O)C=CC2=O)CC1]

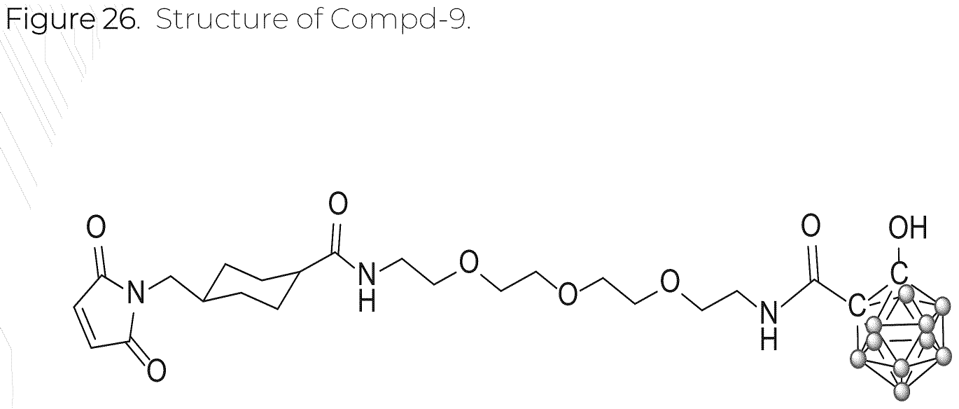

### (See, FIG. 26).

### Example 4: Synthesis of MC-PEG₃-salborin-Compd-11.

Using the general scheme set forth in Example 1, Compd-11 was synthesized in the following manner. Briefly, in a round bottom flask charged with a stir bar, a mixture of N-(2-(2-(2-(2-aminoethoxy)ethoxy)ethoxy)ethyl)salbamide (4.) (1.1 eq, 1 mol), 2-maleimidoacetic acid (10.) (1.0 eq, 1 mol), EDC—HCl (1.1 eq, 1 mol), HOBt·H₂O (1.1 eq, 1 mol) and TEA (1.1 eq, 1 mol) was dissolved in anhydrous DMF (1 mL/mol). The reaction mixture was kept overnight and concurrently monitored by LC/MS. On completion, the reaction mixture was diluted with brine (5 mL) and extracted with ethyl acetate (5 mL×3). The organic layer was dried over sodium sulfate and removed under reduced pressure. The crude mixture was subjected to prep-LC (10 to 80% ACN/H₂O) resulting in mal-amido-PEG₃-salborin (11.) as a translucent solid.

The resulting synthesis provided Compd-11 which contains the following chemical structure:

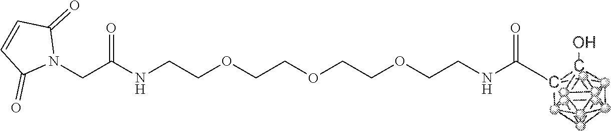 [SMILES: CC(=O)NCCOCCOCCOCCNC(=O)CN1C(=O)C=CC1=O]

### (See, FIG. 27).

### Example 5: Synthesis of Compd-14 Intermediates.

The synthesis of Compd-14 was performed using the following Scheme:

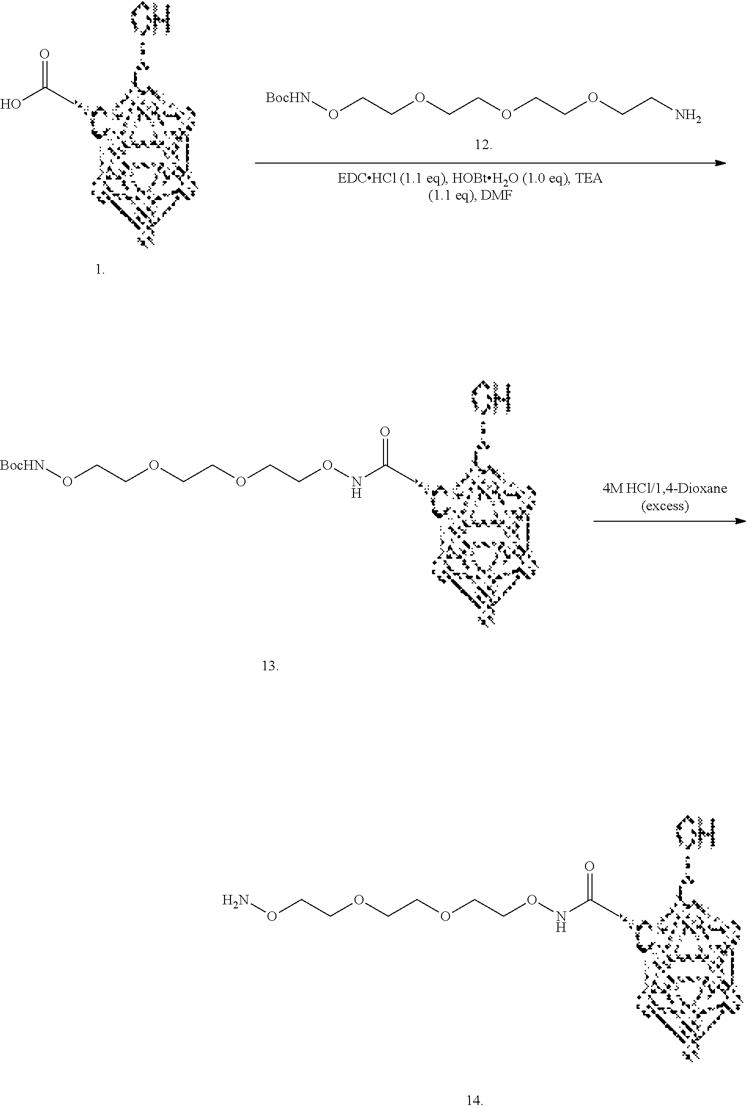

Briefly, to obtain aminooxy-PEG₃-salborin (14.), amide coupling of salborin (1.) and tert-butyl(2-(2-(2-(2-aminoethoxy)ethoxy)ethoxy)ethoxy)carbamate (12.) with EDC.HCl, HOBt·H₂O and TEA in anhydrous DMF solvent afforded the aminooxy-carbamate-PEG₃-salborin (13.) intermediate. Boc-deprotection of intermediate 13 with 4M HCl/1,4-dioxane gave us the target product aminooxy-PEG₃-salborin (14.)

The synthesis of aminooxy-carbamate-PEG₃-salborin (13) was performed in the following manner. Briefly, to a round bottom flask charged with a stir bar, a mixture of tert-butyl (2-(2-(2-(2-aminoethoxy) ethoxy) ethoxy) ethoxy) carbamate (12.) (1.1 eq, 1 mmol), salborin (1.) (1.0 eq, 1 mmol), EDC·HCl (1.1 eq, 1 mmol), HOBt·H₂O (1.1 eq, 1 mmol) and TEA (1.1 eq, 1 mmol) was dissolved in anhydrous DMF (1 mL/mmol). The reaction mixture was kept overnight and concurrently monitored by LC/MS. On completion, the reaction mixture was diluted with brine (5 mL) and extracted with ethyl acetate (5 mL×3). The organic layer was dried over sodium sulfate and removed under reduced pressure. The crude mixture was subjected to prep-LC (10 to 80% ACN/H₂O) resulting in aminooxy-carbamate-PEG₃-salborin (13) as a translucent solid having the following chemical structure:

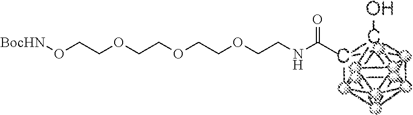 [SMILES: CC(=O)NCCOCCOCCOCCONC(=O)OC(C)(C)C]

### Example 6: Synthesis of aminooxy-PEG₃-salborin-Compd-14

Using the general scheme set forth in Example 5, Compd-14 was synthesized in the following manner. Briefly, in a flame dry round bottom flask charged with a stir bar, aminooxy-carbamate-PEG₃-salborin (13) was dissolved in excess 4M HCl in 1,4-dioxane. The reaction was monitored with LC/MS. On completion it was concentrated under reduced pressure. The crude mixture was subjected to prep-LC (10 to 80% ACN/H₂O) resulting in aminooxy-PEG₃-salborin (14.) as a translucent oil.

The resulting synthesis provided Compd-14, which contains the following chemical structure:

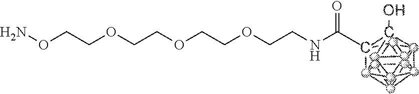 [SMILES: CC(=O)NCCOCCOCCOCCON]

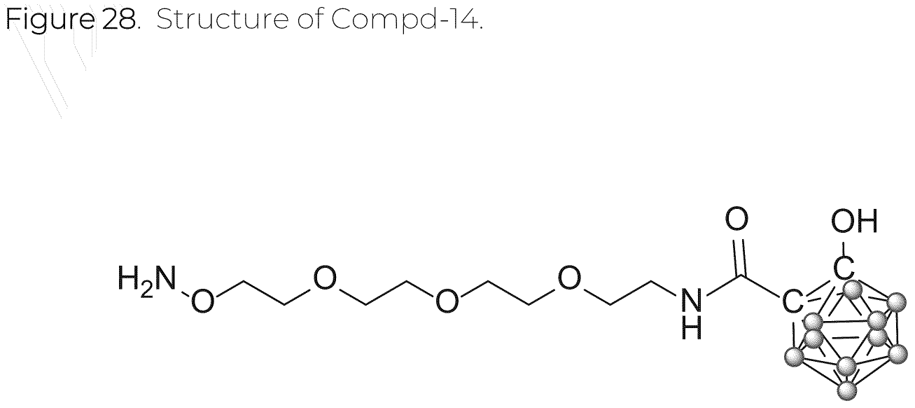

### (See, FIG. 28).

### Example 7: Synthesis of Compd-20 and Compd-21 Intermediates.

The synthesis of Compd-20 and Compd-21 was performed using the following Scheme:

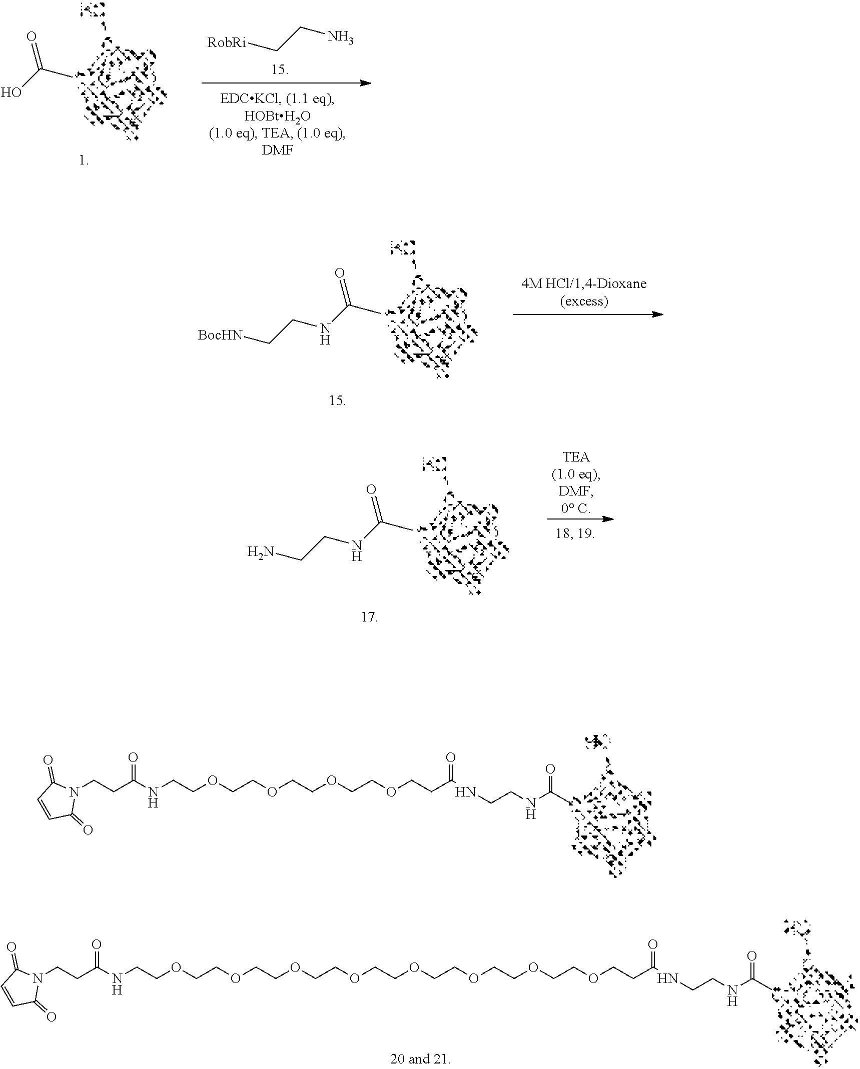

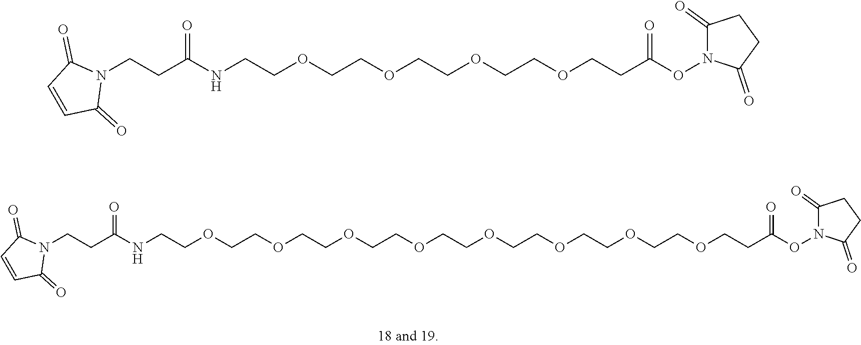 [SMILES: O=C(CCN1C(=O)C=CC1=O)NCCOCCOCCOCCOCCC(=O)ON1C(=O)CCC1=O ; O=C(CCN1C(=O)C=CC1=O)NCCOCCOCCOCCOCCOCCOCCOCCOCCC(=O)ON1C(=O)CCC1=O]

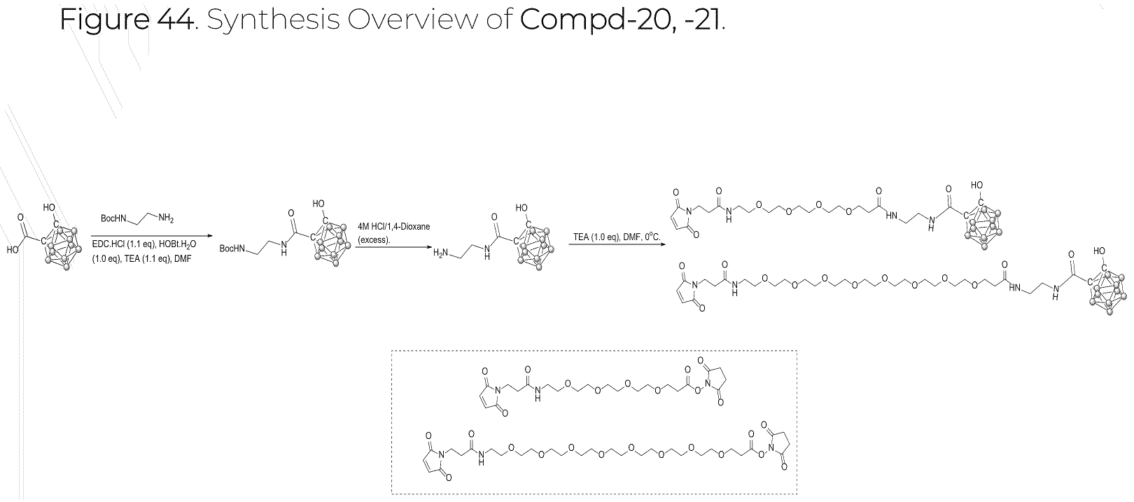

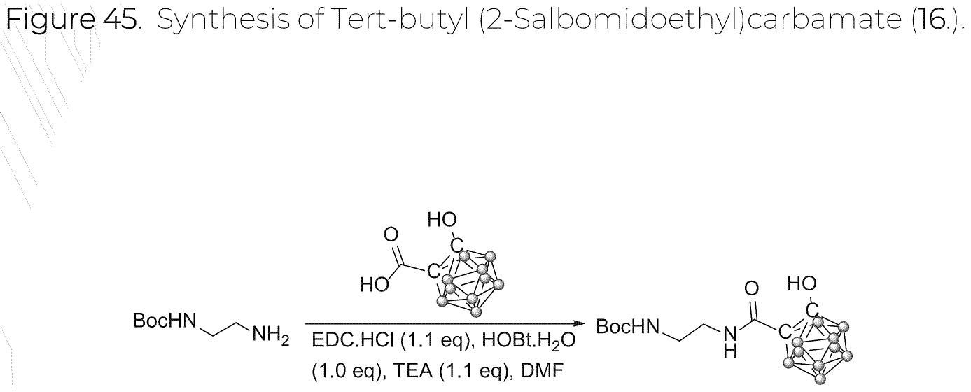

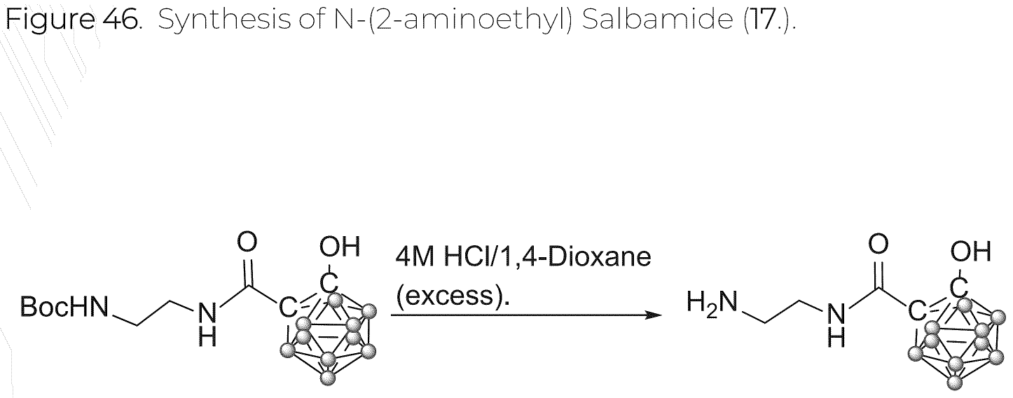

### (See, FIG. 44).

Briefly, commercially available tert-butyl (2-aminoethyl) carbamate (15.) was coupled with salborin (1.) using EDC·HCl, HOBt·H₂O and TEA dissolved in anhydrous DMF to obtain intermediate 16. Boc-deprotection of intermediate 16. with excess 4M HCl in 1,4-dioxane afforded intermediate 17. Coupling intermediate 17. with maleimide-PEGₙ-NHS reagent (18, or 19) in the presence of TEA in DMF at 0° C. afforded target Compd-20 and Compd-21.

Synthesis of ter-butyl (2-salbomidoethyl) carbamate (16.) was performed in the following manner. Briefly, to a round bottom flask charged with a stir bar, a mixture of tert-butyl (2-aminoethyl) carbamate (1.) (1.1 eq, 1 mmol), salborin (2.) (1.0 eq, 1 mmol), EDC.HCl (1.1 eq, 1 mmol), HOBI-H₂O (1.1 eq, 1 mmol) and TEA (1.1 eq, 1 mmol) was dissolved in anhydrous DMF (1 mL/mmol). The reaction mixture was kept overnight and concurrently monitored by LC/MS. On completion, the reaction mixture was diluted with brine (5 mL) and extracted with ethyl acetate (5 ml×3). The organic layer was dried over sodium sulfate and removed under reduced pressure. The crude mixture was subjected to prep-LC (10 to 80% ACN/H₂O) resulting in ter-butyl (2-salbomidoethyl)carbamate (3.) as a white solid having the following chemical structure:

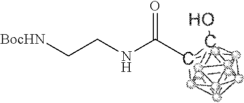

Synthesis of N-(2-aminoethyl) salbamide (17.) was performed in the following manner. Briefly, to a flame dry round bottom flask charged with a stir bar, tert-butyl (2-salbomidoethyl) carbamate (16.) was dissolved in excess 4M HCl in 1,4-dioxane. The reaction was monitored via LC/MS. On completion, it was concentrated under reduced pressure to obtain N-(2-aminoethyl) salbamide (17.) as a white solid:

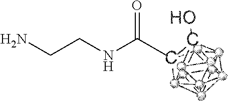

### Example 8: Synthesis of mal-amido-PEGA-EDA-salborin-Compd-20.

Using the general scheme set forth in Example 7, Compd-20 was synthesized in the following manner. Briefly, in a round bottom flask charged with a stir bar, a mixture of N-(2-aminoethyl) salbamide (16.) (1.1 eq, 1 mmol), maleimido-PEGA-NHS ester (18.) (1.0 eq, 1 mmol) and TEA (1.1 eq, 1 mmol) were dissolved in anhydrous DMF (1 mL/mmol) at zero degrees. The reaction mixture was monitored by LC/MS. On completion, the reaction mixture was diluted with distilled H₂O (2 mL) and extracted with ethyl acetate (3 mL×3). The organic layer was dried over sodium sulfate and removed under reduced pressure. The crude mixture was subjected to prep-LC (10 to 80% ACN/H₂O) resulting in mal-amido-PEG4-EDA-salborin (20.) as clear liquids.

The resulting synthesis provided Compd-20 having the following chemical structure:

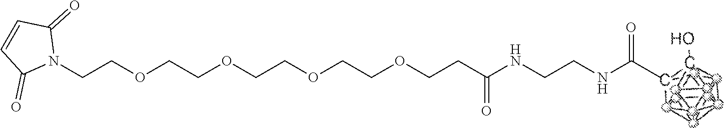 [SMILES: CC(=O)NCCNC(=O)CCOCCOCCOCCOCCN1C(=O)C=CC1=O]

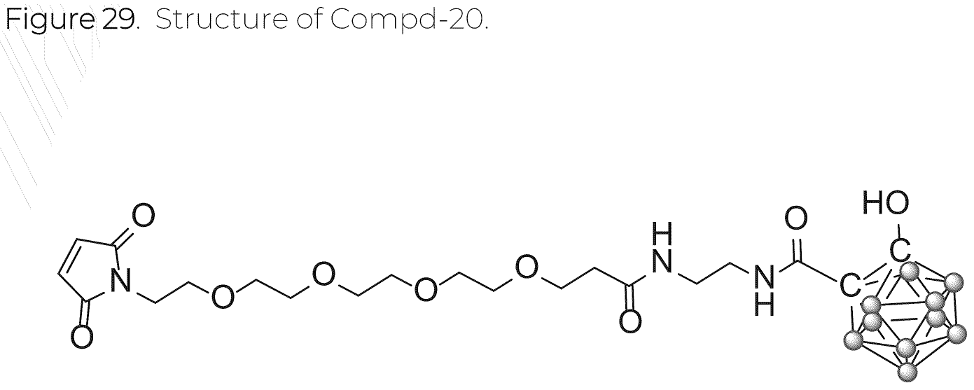

### (See, FIG. 29).

### Example 9: Synthesis of mal-amido-PEG:-EDA-salborin Compd-21.

Using the general scheme set forth in Example 7, Compd-21 was synthesized in the following manner. Briefly, in a round bottom flask charged with a stir bar, a mixture of N-(2-aminoethyl) salbamide (16.) (1.1 eq, 1 mmol), mal-amido-PEG₈-NHS ester (19.) (1.0 eq, 1 mmol) and TEA (1.1 eq, 1 mmol) were dissolved in anhydrous DMF (1 mL/mmol) at zero degrees. The reaction mixture was monitored by LC/MS. On completion, the reaction mixture was diluted with distilled H₂O (2 mL) and extracted with ethyl acetate (3 mL×3). The organic layer was dried over sodium sulfate and removed under reduced pressure. The crude mixture was subjected to prep-LC (10 to 80% ACN/H₂O) resulting in mal-amido-PEG₈-EDA-salborin (21.) as clear liquids.

The resulting synthesis provided Compd-21:

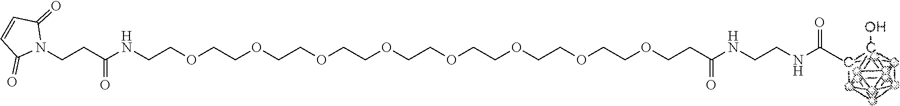 [SMILES: CC(=O)NCCNC(=O)CCOCCOCCOCCOCCOCCOCCOCCOCCNC(=O)CCN1C(=O)C=CC1=O]

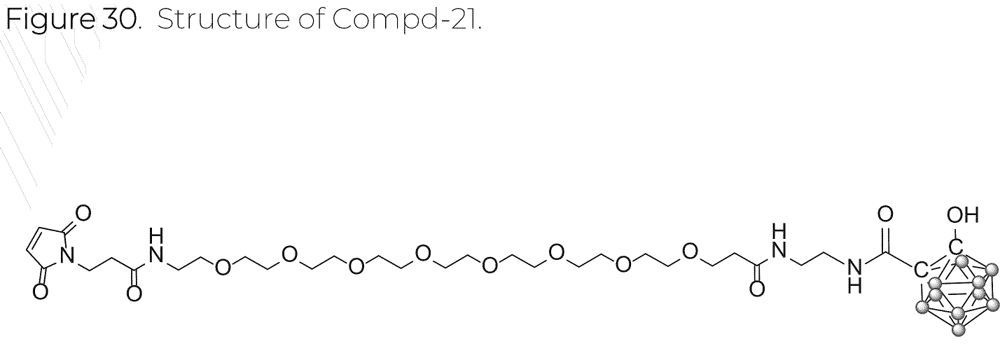

### (See, FIG. 30).

### Example 10: Synthesis of Compd-25 Intermediates.

The synthesis of Compd-25 was performed using the following Scheme:

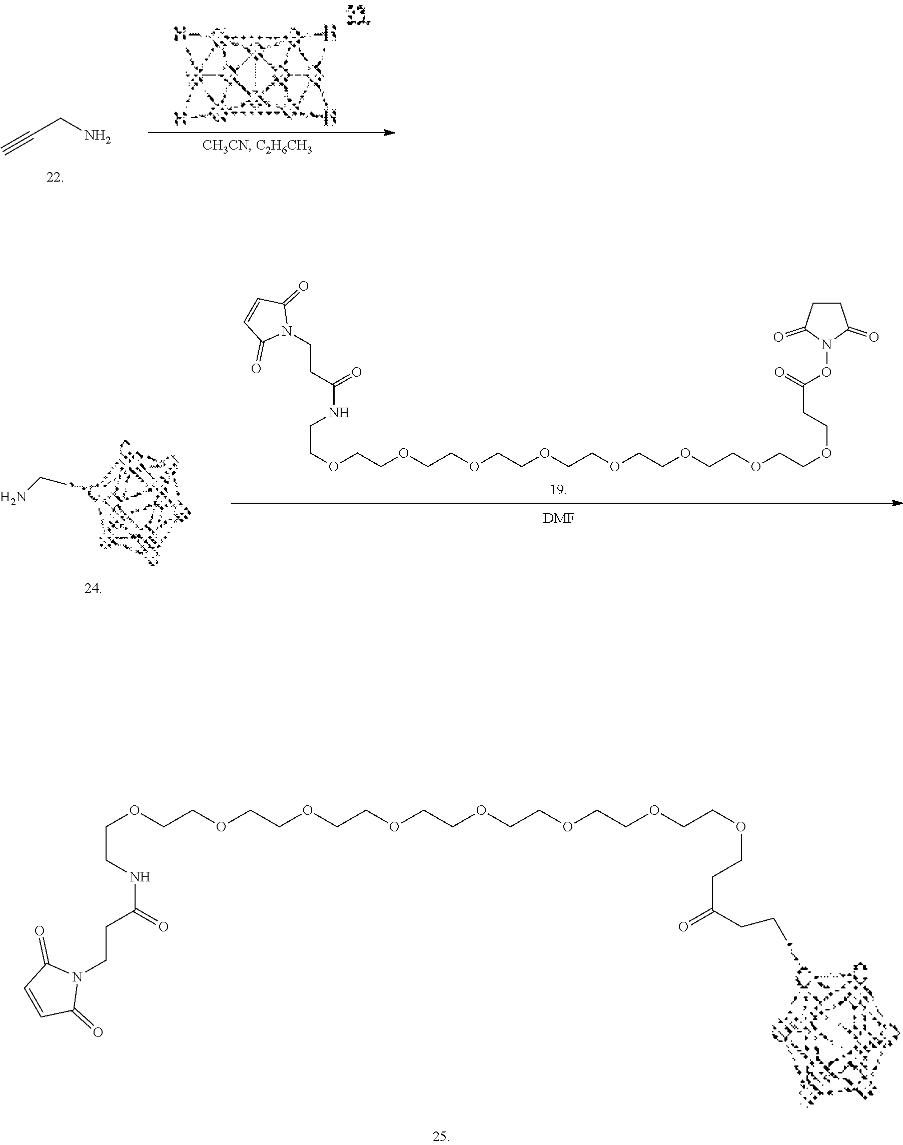

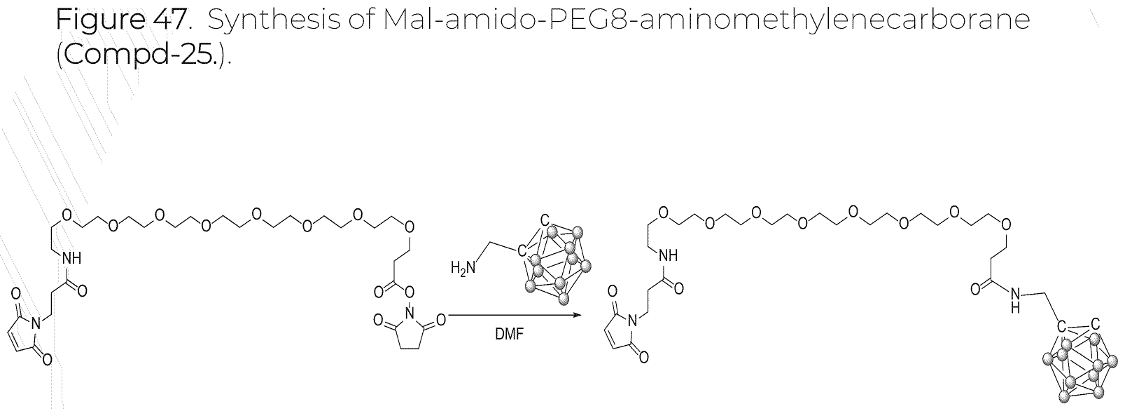

### (See, FIG. 47).

Briefly, synthesis of aminocarboranylmethane (24.) was performed in the following manner. To a round bottom flask that has been flamed-dried, 1.6 g of decaborane (23.) was added and dissolved in 16.8 mL of anhydrous acetonitrile. After two (2) hours of refluxing, a yellow solid was formed, to which the reaction was concentrated down and backfilled with inert gas. Then, 32 mL of dry toluene was added back into the flask. A solution of 1 g of 2-propynylamine (22.) dissolved in 8 mL of dry toluene was then slowly added to the solution. The reaction was refluxed at 110° C. for three (3) hours, which the reaction went to completion as observed by LC/MS. The reaction was then quenched with 2 mL of methanol and allowed to reflux for an additional hour. The reaction mixture was then concentrated and subjected to flash column chromatography to afford aminocarboranylmethane (24.) as a white solid having the following chemical structure:

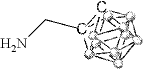

### Example 11: Synthesis of mal-amido-PEG₈-aminocarboranylmethane-Compd-25

Using the general scheme set forth in Example 10, Compd-25 was synthesized in the following manner. Briefly, to a solution of 3 mL of dimethylformamide (DMF) at room temperature was added aminocarboranylmethane (1 eq, 0.5 mmol) (24.) and mal-amido-PEG₈-NHS (1.1 eq, 0.6 mmol) (16.). After stirring for four hours, the reaction was complete as observed by LC/MS. The solvent was then removed under reduced pressure and the concentrate purified via flash column chromatography (FCC) to yield the target material mal-amido-PEG₈-aminocarboranylmethane (25.) as a brown semi solid. The resulting synthesis provided Compd-25 having the following chemical structure:

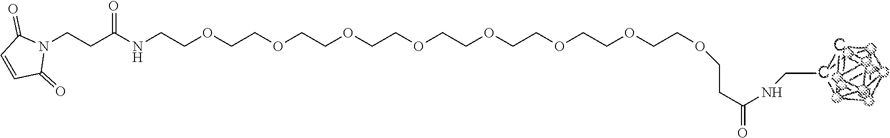 [SMILES: CCNC(=O)CCOCCOCCOCCOCCOCCOCCOCCOCCNC(=O)CCN1C(=O)C=CC1=O]

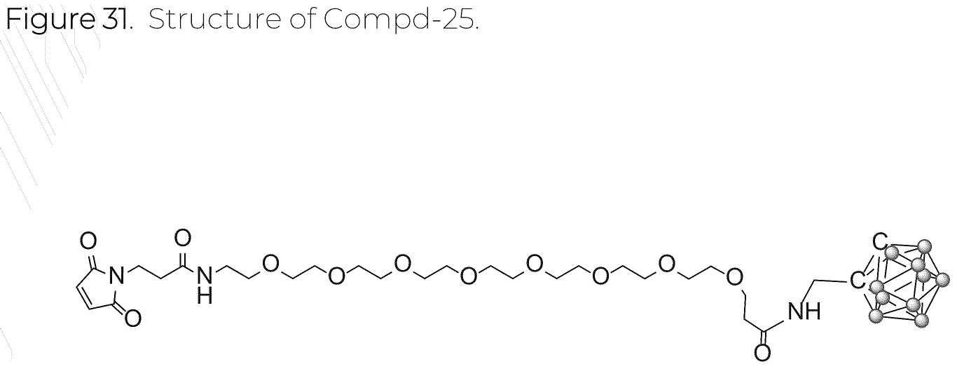

### (See, FIG. 31).

### Example 12: Synthesis of Compd-27 and Compd-31 Intermediates.

The synthesis of Compd-27 and Compd-31 was performed using the following Scheme:

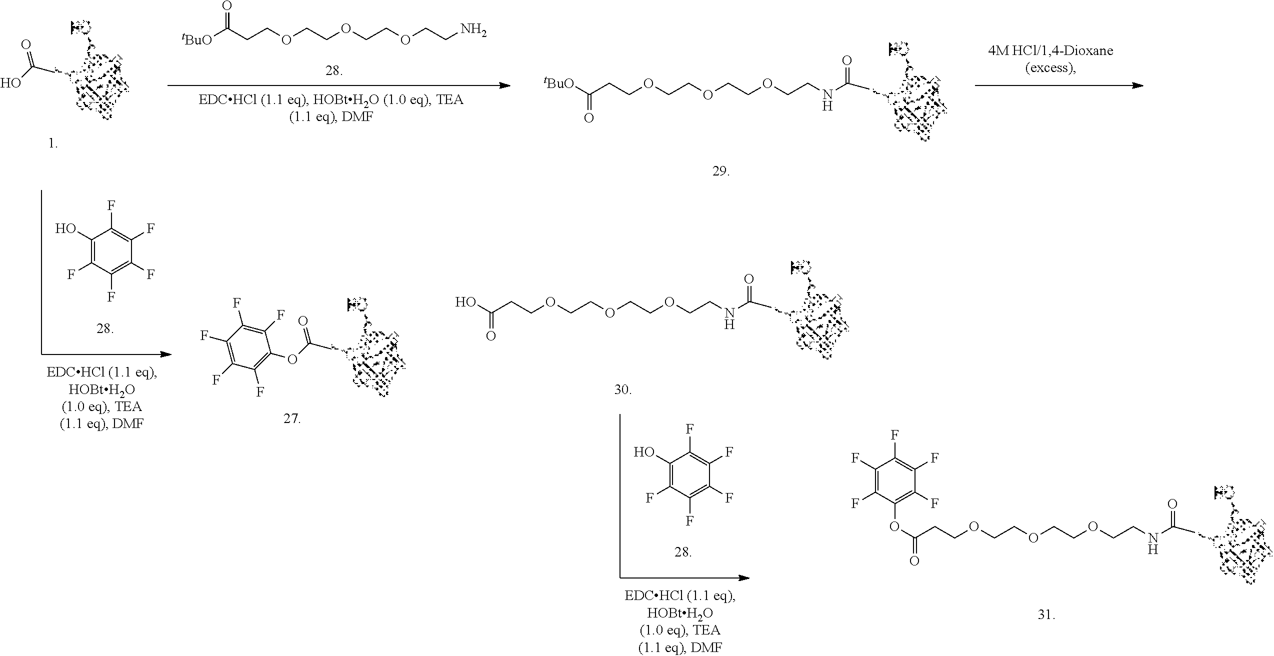

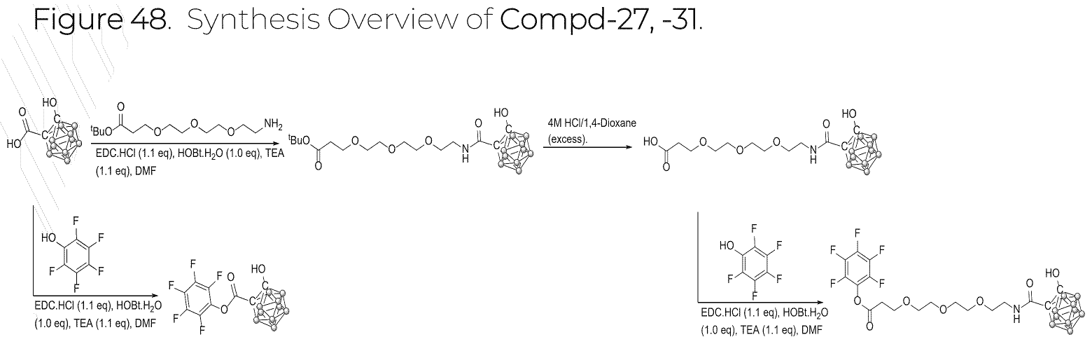

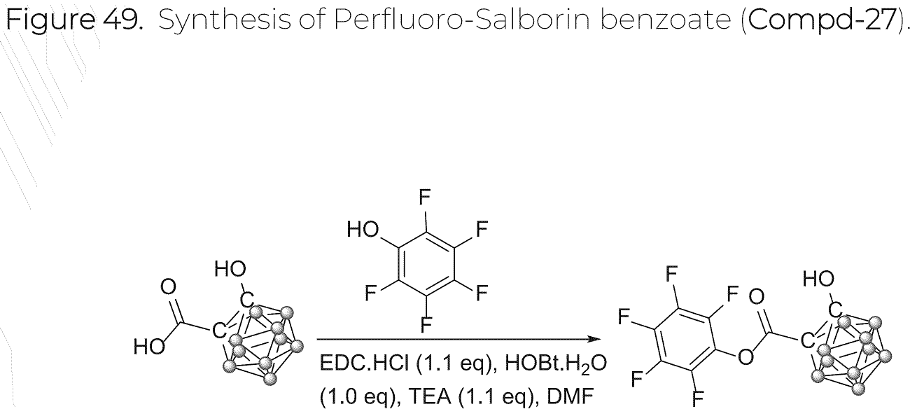

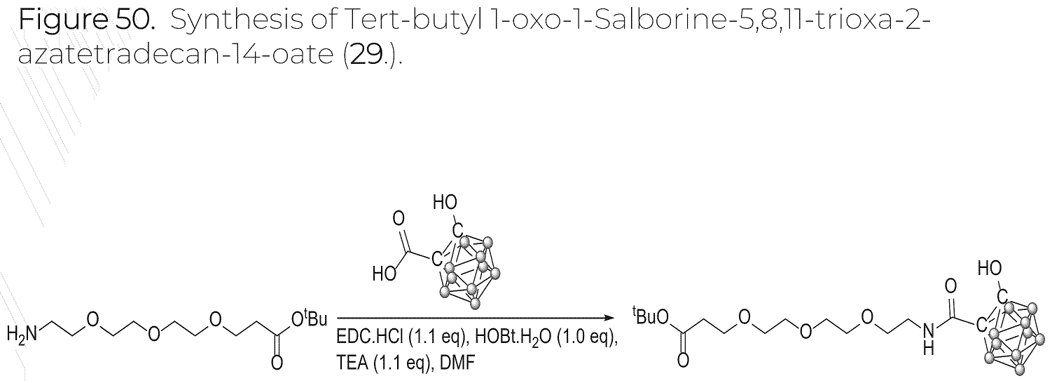

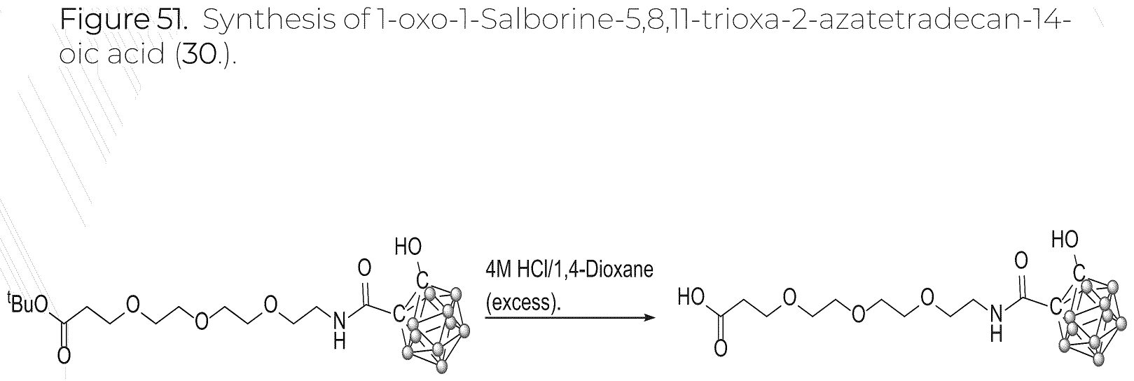

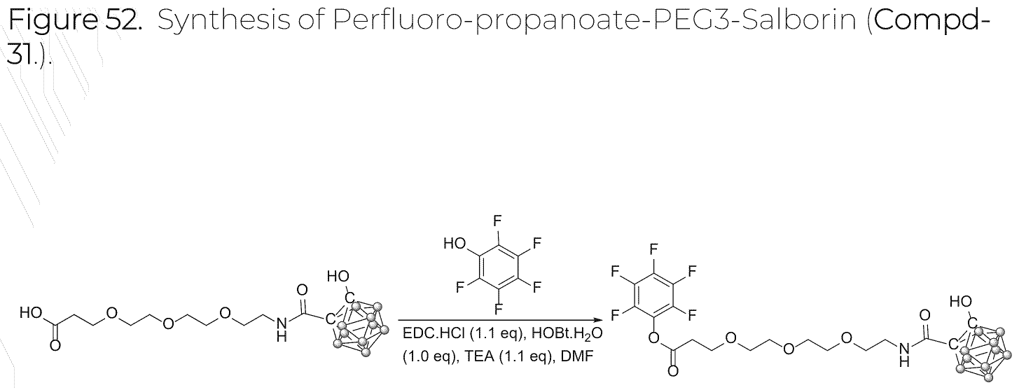

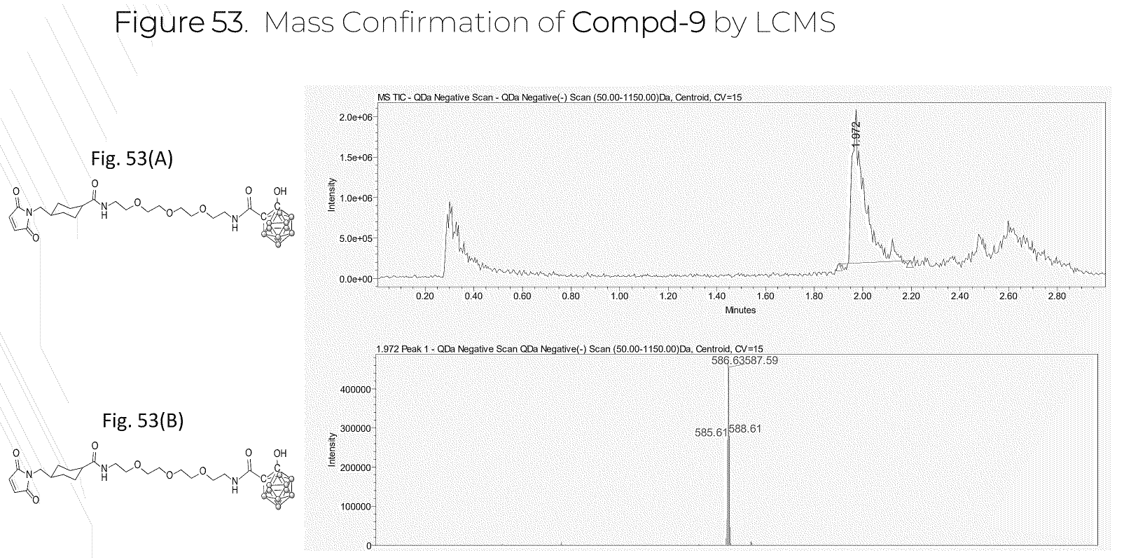

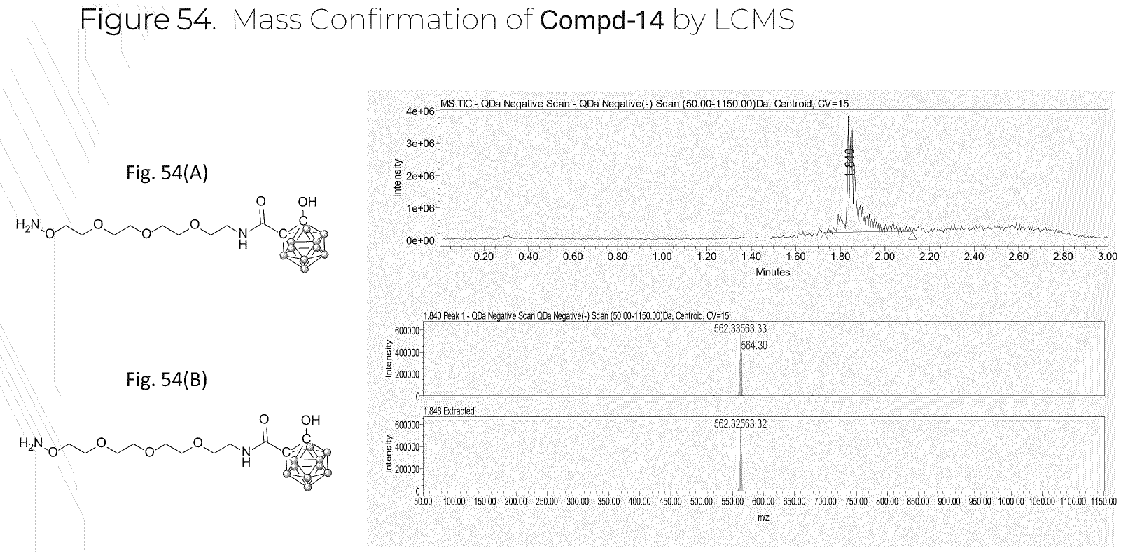

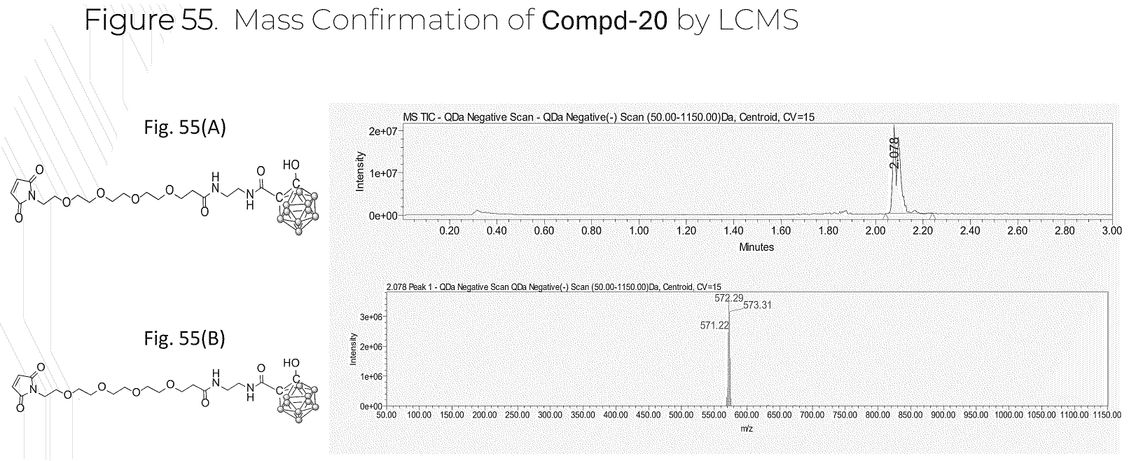

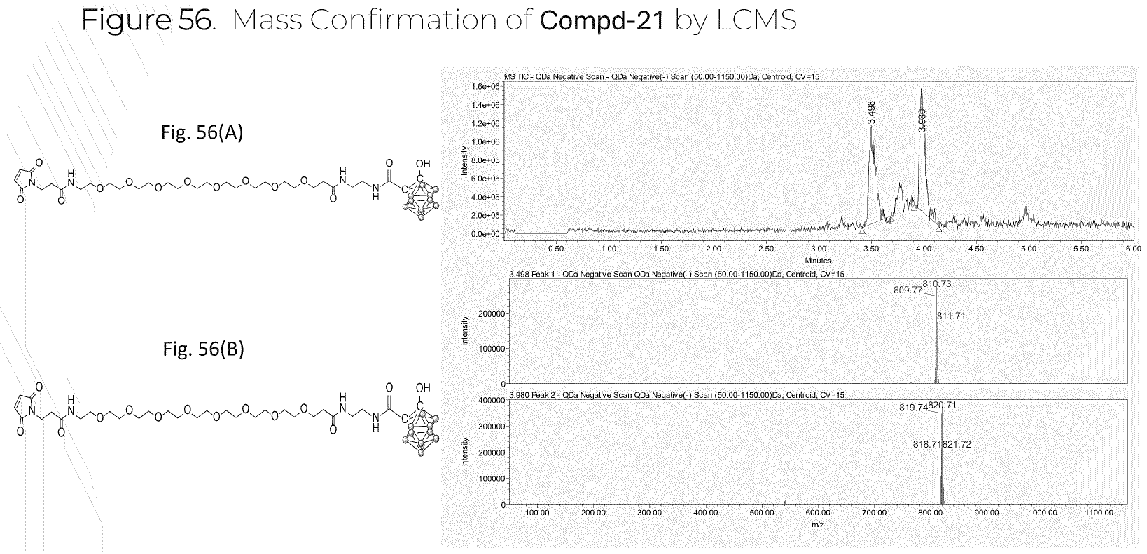

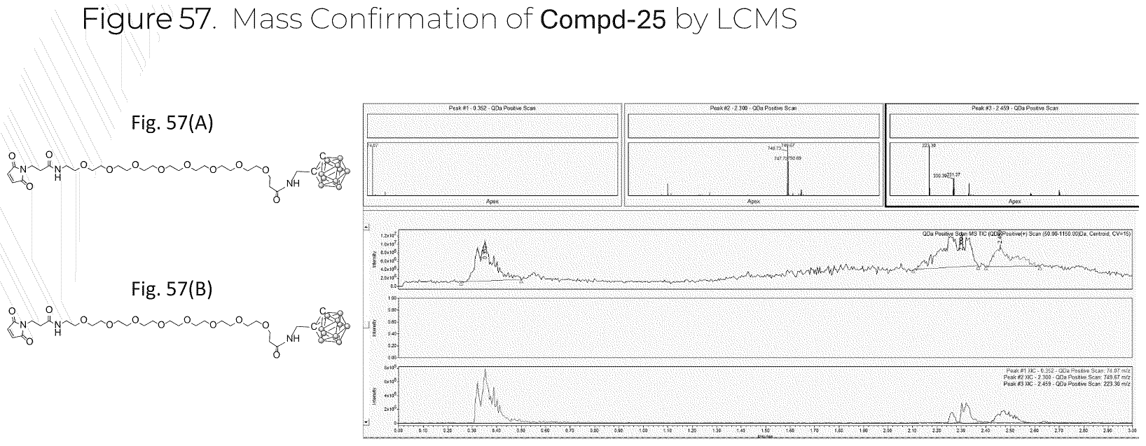

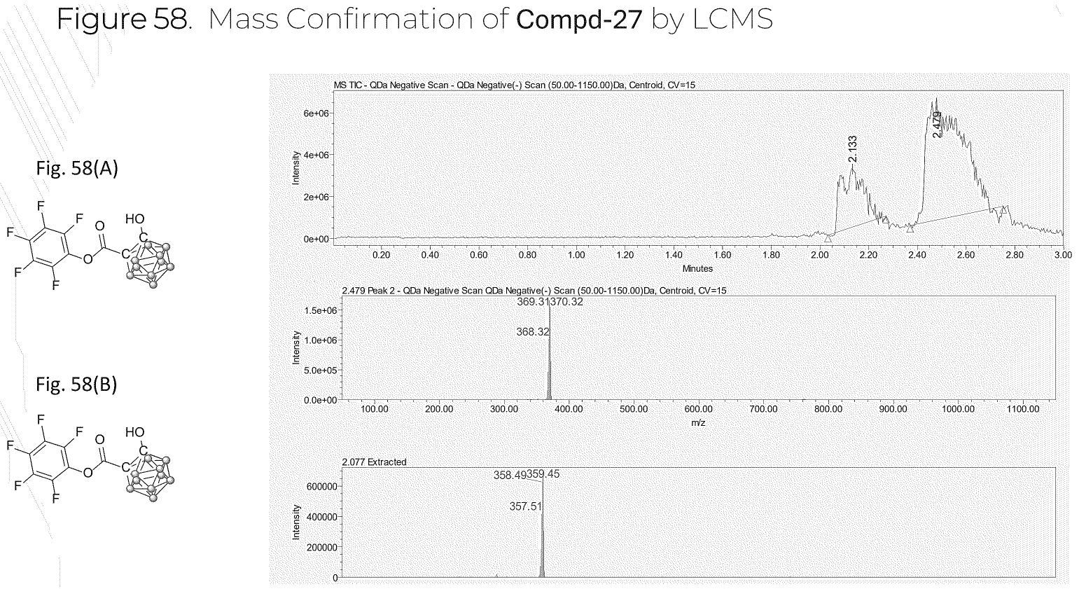

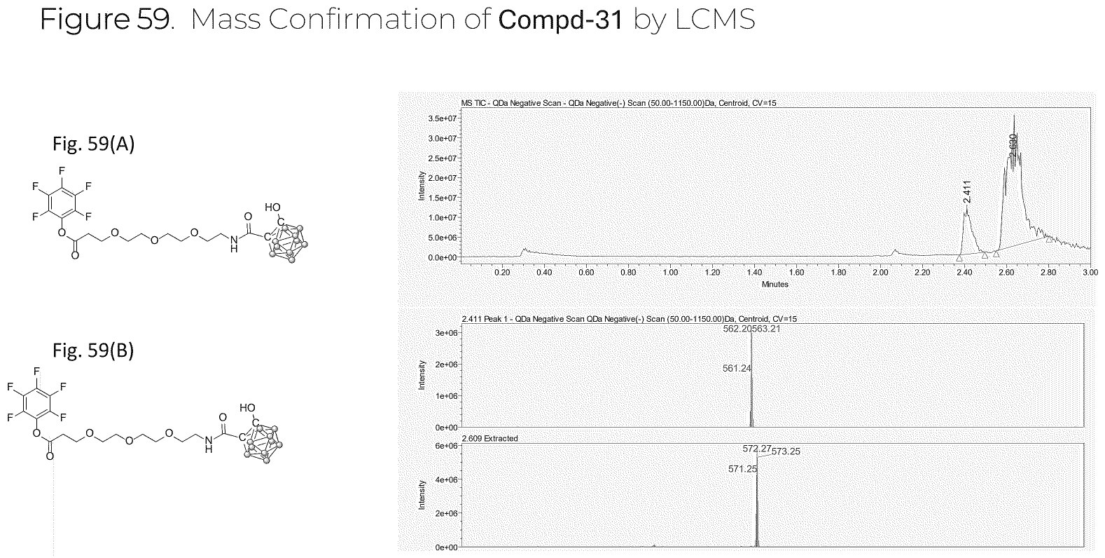

### (See, FIG. 48).

Briefly, salborin perfluorophenyl ester (27.) was obtained through EDC.HCl, HOBt·H₂O, TEA coupling of salborin (1.) and pentafluorophenol (26.) in anhydrous DMF. Concurrently, similar reaction conditions were applied to tert-butyl 3-(2-(2-(2-aminoethoxy) ethoxy) ethoxy) propanoate (28.) and salborin (1.) to get intermediate 29. Intermediate 29. was subjected to acid-mediated hydrolysis with 4M HCl/1,4-dioxane to obtain salborin-PEG₃-acid 30. Reagents, EDC·HCl, HOBt·H₂O, TEA facilitated coupling of salborin-PEG₃-acetic acid intermediate 30. and pentafluorophenol (26.) in anhydrous DMF gave the target compound perfluorophenyl salborin-PEG3-propanoate (31.).

Synthesis of tert-butyl 1-oxo-1-salborin-5,8,11-trioxa-2-azatetradecan-14-oate (29.) was performed in the following manner. Briefly, to a round bottom flask charged with a stir bar, a mixture of tert-butyl 3-(2-(2-(2-aminoethoxy)ethoxy) ethoxy) propanoate (28.) (1.1 eq, 1 mmol), salborin (2.) (1.0 eq, 1 mmol), EDC.HCl (1.1 eq, 1 mmol), HOBt-H₂O (1.1 eq, 1 mmol) and TEA (1.1 eq, 1 mmol) was dissolved in anhydrous DMF (1 mL/mmol). The reaction mixture was kept overnight and concurrently monitored by LC/MS. On completion, the reaction mixture was diluted with brine (5 mL) and extracted with ethyl acetate (5 ml×3). The organic layer was dried over sodium sulfate and removed under reduced pressure. The crude mixture was subjected to prep-LC (10 to 80% ACN/H₂O) resulting in tert-butyl butyl 1-oxo-1-salborin-5,8,11-trioxa-2-azatetradecan-14-oate (29.) as pure translucent liquid having the following chemical structure:

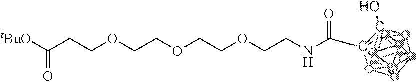 [SMILES: CC(=O)NCCOCCOCCOCCC(=O)OC(C)(C)C]

Synthesis of 1-oxo-1-salborin-5,8,11-trioxa-2-azatetradecan-14-oic acid (30.) was performed in the following manner. Briefly, to a flame-dried round bottom flask charged with a stir bar, tert-butyl 1-oxo-1-salborin-5,8,11-trioxa-2-azatetradecan-14-oate (29.) was dissolved in excess 4M HCl in 1,4-dioxane. The reaction was monitored with LC/MS. On completion it was subjected to reduced pressure under vacuum to obtain 1-oxo-1-salborin-5,8,11-trioxa-2-azatetradecan-14-oic acid (30.) as a pure, translucent oil having the following chemical structure:

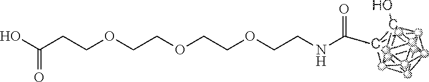 [SMILES: CC(=O)NCCOCCOCCOCCC(=O)O]

### Example 13: Synthesis of salborin perfluorophenyl ester-Compd-27.

Using the general scheme set forth in Example 12, Compd-27 was synthesized in the following manner. Briefly, to a round bottom flask charged with a stir bar, a mixture of salborin (2.) (1.0 eq, 1 mmol), 2,3,4,5,6-pentafluorophenol (26.) (1.1 eq, 1 mmol), EDC.HCl (1.1 eq, 1 mmol), HOBt.H₂O (1.1 eq, 1 mmol) and TEA (1.1 eq, 1 mmol) were dissolved in anhydrous DMF (1 mL/mmol). The reaction mixture was kept overnight and concurrently monitored by LC/MS. On completion, the reaction mixture was diluted with brine (5 mL) and extracted with ethyl acetate (5 ml×3). The organic layer was dried over sodium sulfate and removed under reduced pressure. The crude mixture was subjected to prep-LC (10 to 80% ACN/H₂O) resulting in salborin perfluorophenyl ester (27.) as a pure orange solid.

The resulting synthesis provided Compd-27 having the following chemical structure:

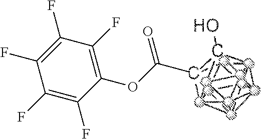

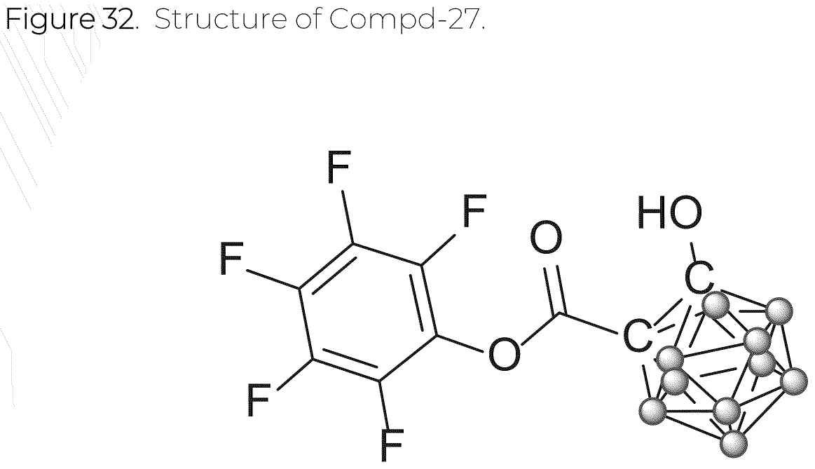

### (See, FIG. 32).

### Example 14: Synthesis of perfluorophenyl salborin-PEG;-propanoate-Compd-31.

Using the general scheme set forth in Example 12, Compd-31 was synthesized in the following manner. Briefly, in a round bottom flask charged with a stir bar, a mixture of 1-oxo-1-salborin-5,8,11-trioxa-2-azatetradecan-14-oic acid (27.) (1.0 eq, 1 mmol), 2,3,4,5,6-pentafluorophenol (23.) (1.1 eq, 1 mmol), EDC.HCl (1.1 eq, 1 mmol), HOBt·H₂O (1.1 eq, 1 mmol) and TEA (1.1 eq, 1 mmol) were dissolved in anhydrous DMF (1 mL/mmol). The reaction mixture was kept overnight and concurrently monitored by LC/MS. On completion, the reaction mixture was diluted with brine (5 ml) and extracted with ethyl acetate (5 mL×3). The organic layer was dried over sodium sulfate and removed under reduced pressure on vacuo. The crude mixture was subjected to prep-LC (10 to 80% ACN/H₂O) resulting in perfluorophenyl salborin-PEG₃-propanoate (31.) as pure crystal solid.

The resulting synthesis provided Compd-31 with the following chemical structure:

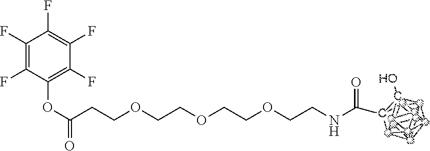 [SMILES: CC(=O)NCCOCCOCCOCCC(=O)Oc1c(F)c(F)c(F)c(F)c1F]

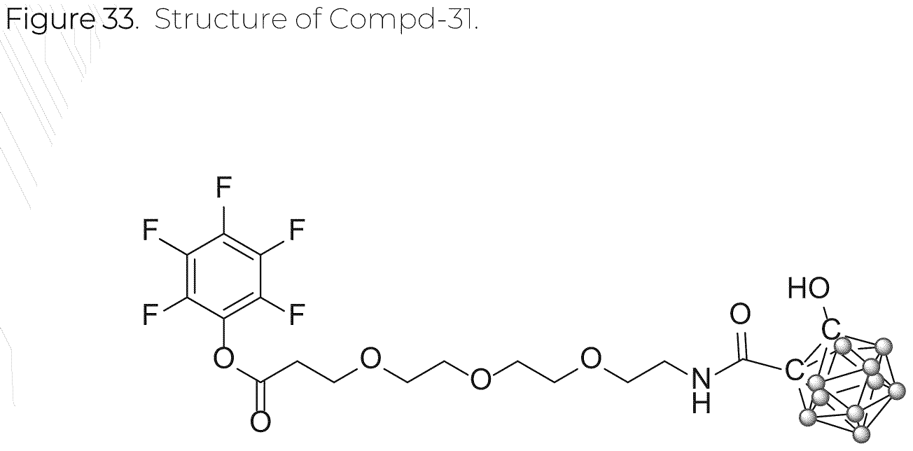

### (See, FIG. 33).

### Example 15: Human Clinical Trials for the Treatment of Human Carcinomas Through the Use of ABCs loaded with BELs.

ABCs loaded with BELs are used in accordance with the present invention which specifically accumulate in a tumor cell, and are used in the treatment of certain tumors and other immunological disorders and/or other diseases. In connection with each of these indications, two clinical approaches are successfully pursued.
I.) Adjunctive therapy: In adjunctive therapy, patients are treated with ABCs loaded with BELs in combination with a chemotherapeutic or pharmaceutical or biopharmaceutical agent or a combination thereof. Primary cancer targets are treated under standard protocols by the addition of ABCs loaded with BELs and then irradiated. Protocol designs address effectiveness as assessed by the following examples, including but not limited to, reduction in tumor mass of primary or metastatic lesions, increased progression free survival, overall survival, improvement of patient's health, disease stabilization, as well as the ability to reduce usual doses of standard chemotherapy and other biologic agents. These dosage reductions allow additional and/or prolonged therapy by reducing dose-related toxicity of the chemotherapeutic or biologic agent.
II.) Monotherapy: In connection with the use of the ABCs loaded with BELs in monotherapy of tumors, the ABCs loaded with BELs are administered to patients without a chemotherapeutic or pharmaceutical or biological agent. In one embodiment, monotherapy is conducted clinically in end-stage cancer patients with extensive metastatic disease. Protocol designs address effectiveness as assessed by the following examples, including but not limited to, reduction in tumor mass of primary or metastatic lesions, increased progression free survival, overall survival, improvement of patient's health, disease stabilization, as well as the ability to reduce usual doses of standard chemotherapy and other biologic agents,

### Dosage

Dosage regimens may be adjusted to provide the optimum desired response. For example, a single ABC injection of loaded BELs may be administered, several divided doses may be administered over time, or the dose may be proportionally reduced or increased as indicated by the exigencies of the therapeutic situation. “Dosage Unit Form” as used herein refers to physically discrete units suited as unitary dosages for the mammalian subjects to be treated; each unit containing a predetermined quantity of active compound calculated to produce the desired therapeutic effect in association with the required pharmaceutical carrier. The specification for the dosage unit forms of the invention are dictated by and directly dependent on (a) the unique characteristics of the ABC loaded with the BEL, the individual mechanics of the irradiation mechanism (reactor) and the particular therapeutic or prophylactic effect to be achieved, and (b) the limitations inherent in the art of compounding such an compound for the treatment of sensitivity in individuals.

### Clinical Development Plan (CDP)

The CDP follows and develops treatments of using ABCs loaded with BELs which are then irradiated using Neutron Capture Therapy in connection with adjunctive therapy or monotherapy. Trials initially demonstrate safety and thereafter confirm efficacy in repeat doses. Trials are open label comparing standard chemotherapy with standard therapy plus ABCs loaded with BELs which are then irradiated using Boron Neutron Capture Therapy. As will be appreciated, one non-limiting criteria that can be utilized in connection with enrollment of patients is concentration of BELs in a tumor as determined by standard detection methods known in the art.

The present invention is not to be limited in scope by the embodiments disclosed herein, which are intended as single illustrations of individual aspects of the invention, and any that are functionally equivalent are within the scope of the invention. Various modifications to the models, methods, and life cycle methodology of the invention, in addition to those described herein, will become apparent to those skilled in the art from the foregoing description and teachings, and are similarly intended to fall within the scope of the invention. Such modifications or other embodiments can be practiced without departing from the true scope and spirit of the invention.

TABLE I
Examples of Boron Clusters used to
synthesize Boron Enriched Linkers.

| Number of Vertices | Polyhedron |
|---|---|
| 4 | Tetrahedron |
| 5 | Trigonal bipyramid |
| 6 | Octahedron |
| 7 | Pentagonal bipyramid |
| 8 | D_{2d} (trigonal) |
| | dodecahedron |
| 9 | Tricapped trigonal prim |
| 10 | Bicapped square |
| | antiprism |
| 11 | Edge-contracted |
| | icosahedron |
| 12 | Icosahedron |

TABLE I(A)

| Electron | | |
|---|---|---|
| count | Name | Predicted structure |
| 4n − 2 | Bicapped | n − 2 |
| | closo | vertex closo polyhedron |
| | | with 2 capped |
| | | (augmented) faces |
| 4n | Capped | n − 1 |
| | closo | vertex closo polyhedron |
| | | with 1 face capped |
| 4n + 2 | closo | closo polyhedron |
| | | with n vertices |
| 4n + 4 | nido | n + 1 |
| | | vertex closo polyhedron |
| | | with 1 missing vertex |
| 4n + 6 | arachno | n + 2 |
| | | vertex closo polyhedron |
| | | with 2 missing vertices |
| 4n + 8 | hypho | n + 3 |
| | | vertex closo polyhedron |
| | | with 3 missing vertices |
| 4n + 10 | klado | n + 4 |
| | | vertex closo polyhedron |
| | | with 4 missing vertices |

1. A compound comprising a chemical structure as follows:

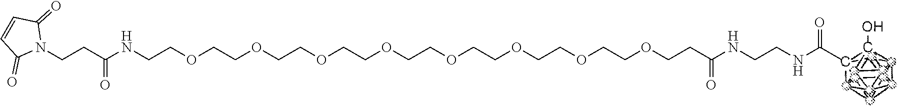 [SMILES: CC(=O)NCCNC(=O)CCOCCOCCOCCOCCOCCOCCOCCOCCNC(=O)CCN1C(=O)C=CC1=O]

2. The compound of claim 1, wherein the compound is conjugated to an antibody.

3. A method of producing a compound of claim 1.

4. A kit comprising the compound of claim 1.

5. A kit comprising the compound of claim 2.

6. A compound comprising a chemical structure as follows:

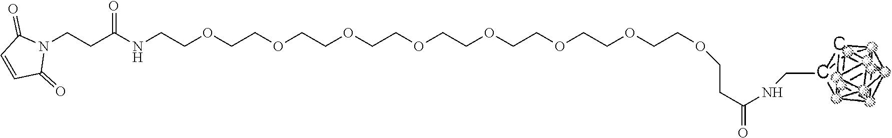 [SMILES: CCNC(=O)CCOCCOCCOCCOCCOCCOCCOCCOCCNC(=O)CCN1C(=O)C=CC1=O]

7. The compound of claim 5, wherein the compound is conjugated to an antibody.

8. A method of producing a compound of claim 5.

9. A kit comprising the compound of claim 5.

10. A method of producing an antibody-drug-conjugate (ABC) by the process comprising,
a. activation of a lysine side chain(s) on a poly-D,L-lysine polymer by grafting a linker terminating in benzaldehyde or thiol or any thiol revealing linker;
b. ligation of the activated poly-D,L-lysine and mAb to create a pre-ABC;
c. purifying the pre-ABC from free poly-D,L-lysine by a mixed-mode chromatography, whereby the mixed-mode chromatography comprises ceramic hydroxyapatite;
d. conjugation of said pre-ABC to a boron enriched linker

11. The method of claim 62, wherein the BEL is a carborane-based boron enriched PEG linker.

12. The method of claim 62, wherein the BEL utilizes oxyamine terminated linking chemistry.

13. The method of claim 62, wherein the BEL utilizes maleimide terminated linking chemistry

14. The method of claim 62, wherein the mAb binds to EGFR.

15. The method of claim 62, wherein the mAb binds to Her2

16. The method of claim 62 consisting essentially of the of the steps shown in FIG. 1.

17. The method of claim 10, whereby the conjugate of the ABC is the compound set forth in claim 1.

18. The method of claim 10, whereby the conjugate of the ABC is the compound set forth in claim 5.

19. The method of claim 10, wherein said pre-ABC is prepared by ligation Compd-C having the following chemical structure: 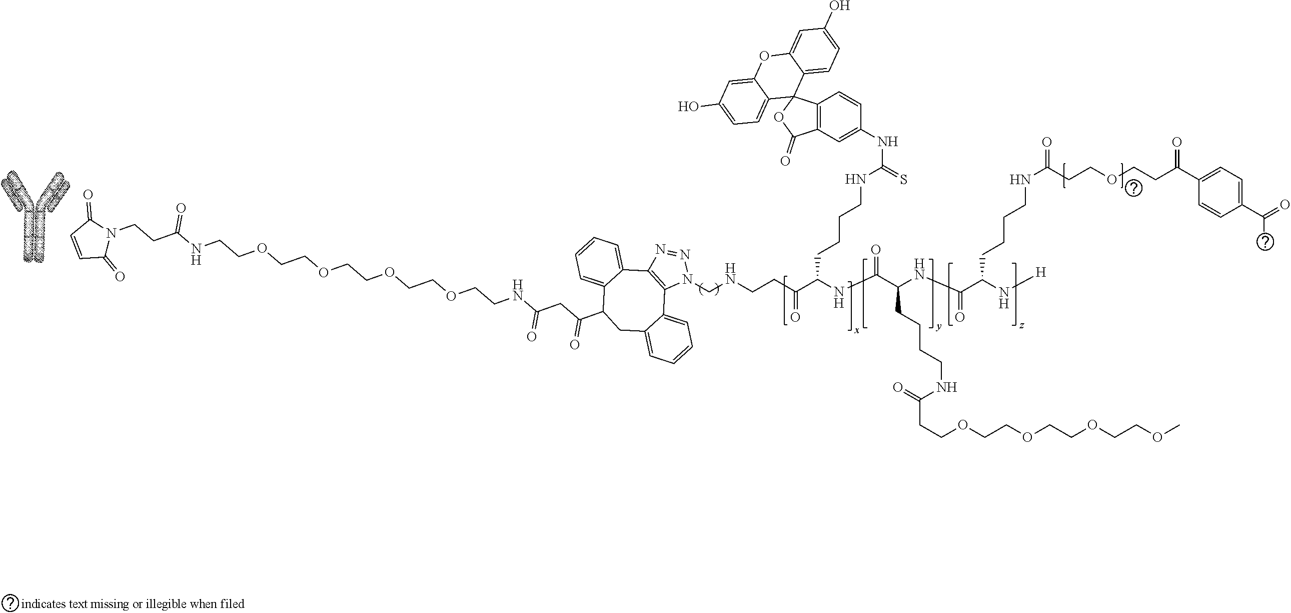
wherein x=1 or 2;
y=1 to 15; and
z=1 to 15.

20. The method of claim 10, wherein a buffer exchange is performed by tangential flow filtration (TFF).
